(19) United States
(12) Patent Application Publication
(10) Pub. No.: US 2012/0095022 A1
(43) Pub. Date: Apr. 19, 2012

(54) DERIVATIVES OF 10-AMINO-1,2,3,4-TETRAHYDROPYRIDO[2,1-A]ISOINDOL-6(10BH)-ONES, METHOD FOR PREPARATION THEREOF AND THERAPEUTIC USES THEREOF

(75) Inventors: Sylvain Cesar Leonce Routier, Tigy (FR); Gerald Guillaumet, Saint Jean Le Blanc (FR); Rajaa Boulahjar, Casablanca (MA); Laurent Meijer, Roscoff (FR); Matteo Chiurato, Bassano del Grappa (Vl) (IT)

(73) Assignees: UNIVERSITE D'ORLEANS, Orleans Cedex 2 (FR); CENTRE NATIONAL DE LA RECHERCHE SCIENTIFIQUE (C.N.R.S.), Paris (FR)

(21) Appl. No.: 13/256,017

(22) PCT Filed: Mar. 11, 2010
(86) PCT No.: PCT/FR2010/050416

§ 371 Date: Dec. 27, 2011

(30) Foreign Application Priority Data
Mar. 12, 2009 (FR) 09 51551

(51) Int. Cl. A61K 31/497; A61K 31/439; A61K 31/437; C07D 471/20; C07D 471/04; A61P 11/06; A61P 35/00; A61P 25/28; A61P 25/16; A61P 25/00; A61P 31/12; A61P 37/02; A61P 17/00; A61P 17/06; A61K 31/506

(52) U.S. Cl. 514/255.05; 514/275; 514/278; 514/294; 544/230; 544/331; 544/405; 546/18; 546/94

(57) ABSTRACT

The present invention relates to compounds having the following general formula (I):

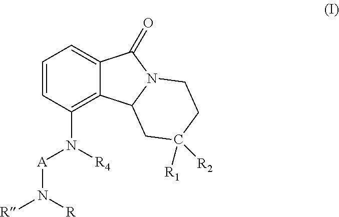

wherein:
   A represents a SO₂ or CX group, X representing O or S;
   R₁, R₂, R″, R₄ represent in particular H,
   R represents in particular an alkyl group or an aryl group,
      as well as to the pharmaceutically acceptable salts thereof, the compound of formula (I) taking the form of a pure stereoisomer or an enantiomer and/or diastereoisomer mixture, including racemic mixtures.

The present invention relates to derivatives of 10-amino-1,2,3,4-tetrahydropyrido[2,1-a]isoindol-6(10bH)-ones and to their preparation methods. It also relates to the therapeutic uses of said novel derivatives notably as inhibitors of kinases.

Kinase proteins catalyze phosphorylation of residues of the serine, threonine and tyrosine type by using ATP or GTP as a phosphate donor. Kinases are presently part of the most investigated biological targets since they are involved in many biological processes. Selective enzymatic inhibition is a preferred strategy for developing novel chemotherapies. Kinases proteins are among the most sought-after biological targets by the pharmaceutical industry. The very large number of kinases has made it difficult to determine the exact role of each of them. They are involved in various processes such as cell growth and differentiation as well as tumoral promotion, organization of the cell cycle or the functioning of neuronal cells. An under- or over-expression of these enzymes has been reported in a large range of neoplastic and pre-neoplastic tissues. During an over-expression, powerful kinase inhibitors may be used as antiproliferative agents.

Considering the importance of these reactions in physiological and cell processes, it is therefore not surprising that dysfunctions of these regulation systems become the cause or the consequence of human affections. In this respect, a large number of pathologies result from the mutation of kinases and phosphatases. Thus, it is currently accepted that abnormal phosphorylations are responsible for the major part of pathologies such as cancers, diabetes, rheumatoid arthritis, Alzheimer's disease, etc. Presently, erlotinib (Tarceva®) and imatinib (Gleevec®) have been launched on the market as kinase inhibitors.

Because of the key role played by cyclin-dependent kinases (CDKs) for entry and progression in the cell cycle, development of pharmacological inhibitors of these enzymes is therefore a major potential therapeutic route for fighting cancer. Very many malfunctions of the CDKs and of their regulators have been described in human tumors. Inhibitors of the enzymatic activity of CDKs may act independently or together with other treatments for limiting tumoral proliferation:

CDKs 1, 2, 4, 5 and 6 are most frequently over-activated or abnormally controlled in tumors. The inhibitors of CDKs then prove to be potential antiproliferative agents stopping the cells in G1 or G2/M.

The inhibitors of CDKs may also be involved in the apoptotic process. Cyclins A, B, D and E and CDKs1 and 2 may play a pro-apoptotic role. The inhibitors of CDKs may then be used in anticancer chemotherapy for potentializing the action of cytotoxic drugs, while ensuring protection of healthy cells.

CDK5 is directly involved in many neurodegenerative processes such as Alzheimer's disease, Parkinson's disease, brain traumas or cerebral vascular strokes. Inhibitors of CDK5 then act as neuroprotective agents.

Finally, CDKs seem to be involved in renal polykystosis, and inflammation processes. Inhibitors of CDKs have very positive effects on animal models of these pathologies.

Glycogen synthase kinase 3 (GSK-3) is a serine/threonine kinase identified originally for its role in the control of glycogen metabolism. Not only is it involved in the indirect transduction of insulin and IGF-1 signals, it is highly present in the brain and a significant body of evidence has accumulated for linking GSK-3 to induced neurotoxicity. This suggests that deregulation of GSK-3 may play a key role in pathogeneses of Alzheimer's disease and consequently GSK-3Beta has appeared as a promising therapeutic target for Alzheimer's disease and other neurodegenerations. Chemically, heterocyclic thiadiazolidinones (TDZD) have been the only molecules proposed as novel drugs for efficient treatment of neurodegenerative disorders where phosphorylation of the tau protein plays a key role like in the case of Alzheimer's disease.

The aim of the present invention is to provide novel inhibitors of CDKs and of GSK-3.

The aim of the present invention is to provide novel inhibitors of CDKs which directly and selectively target said kinases.

More particularly, the aim of the present invention is to provide specific inhibitors of the CDK1, CDK5 and GSK-3 kinases.

Thus the present invention relates to compounds of the general formula (I):

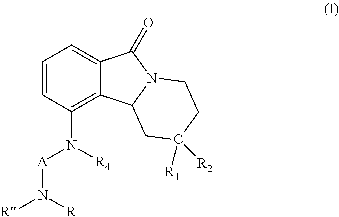

wherein:
A represents a group

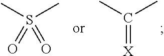

X representing O or S;
   R″ represents H or an alkyl group comprising from 1 to 10 carbon atoms,
   R₁ represents H and R₂ represents an hydrogen atom, an NR₃R′₃ group or an OR₃ group, R₃ and R′₃ representing independently of each other a hydrogen atom or an alkyl group comprising from 1 to 10 carbon atoms; or R₁ and R₂ form together with the carbon atom on which they are bound, a group

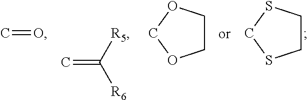

R₅ and R₆ representing independently of each other a hydrogen atom or an alkyl group comprising from 1 to 10 carbon atoms;
   R₄ represents a hydrogen atom or an alkyl group comprising from 1 to 10 carbon atoms;
   R represents an alkyl group comprising from 1 to 10 carbon atoms, an aryl group comprising from 6 to 30 carbon atoms or a cycloalkyl group comprising from 3 to 20 carbon atoms, said aforementioned alkyl, aryl or cycloalkyl groups optionally comprising one or more heteroatoms, notably O, S or N, and may be substituted if need be,
   R notably representing an alkyl group optionally substituted with an amine group,
   or when A is a —CO— group, R and R₄ may form together with the nitrogen atoms on which they are bound, a ring of the following formula (II):

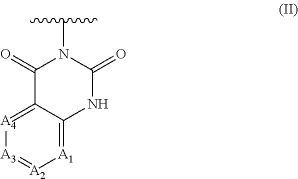

wherein one of the atoms among A₁, A₂, A₃ and A₄ represents N, and the three other atoms among A₁, A₂, A₃ and A₄ represent CH,
   as well as its pharmaceutically acceptable salts,
   said compound of formula (I) being in the form of a pure stereoisomer or in the form of a mixture of enantiomers or diastereoisomers including racemic mixtures.

According to the present invention, the “alkyl” radicals represent saturated hydrocarbon radicals with a linear or branched chain, comprising from 1 to 10 carbon atoms, preferably from 1 to 5 carbon atoms (they may typically be represented by the formula CₙH₂ₙ₊₁, n representing the number of carbon atoms). Mention may notably be made, when they are linear, of methyl, ethyl, propyl, butyl, pentyl, hexyl, octyl, nonyl and decyl radicals. Mention may notably be made, when they are branched or substituted with one or more alkyl radicals, of isopropyl, tert-butyl, 2-ethylhexyl, 2-methylbutyl, 2-methylpentyl, 1-methylpentyl and 3-methylheptyl radicals.

The radical “cycloalkyl” is a saturated or partly unsaturated, nonaromatic, mono-, bi- or tri-cyclic hydrocarbon radical, comprising from 3 to 20 carbon atoms and preferably from 3 to 10 carbon atoms, such as notably cyclopropyl, cyclopentyl, cyclohexyl or adamantyl, as well as the corresponding rings containing one or more unsaturations.

Thus, within the scope of the present invention, the term of “cycloalkyl” also encompasses “heterocycloalkyl” radicals designating saturated or partly unsaturated mono- or bi-cyclic systems with 3 to 8 carbon atoms, comprising one or more heteroatoms selected from N, O or S.

The term of “aryl” designates a mono- or bi-cyclic hydrocarbon aromatic system comprising from 6 to 30, preferably from 6 to 10 carbon atoms. Among aryl radicals, mention may notably be made of the phenyl or naphthyl, radical, more particularly substituted with at least one halogen atom.

When the aryl radical comprises at least one heteroatom, one refers to a “heteroaryl” radical. Thus, the term of “heteroaryl” designates an aromatic system comprising one or more heteroatoms selected from nitrogen, oxygen or sulphur, which is mono- or bi-cyclic, comprising from 5 to 30, and preferably from 5 to 10 carbon atoms. Among heteroaryl radicals, mention may be made of pyrazinyl, thienyl, oxazolyl, furazanyl, pyrrolyl, 1,2,4-thiadiazolyl, naphthyridinyl, pyridazinyl, quinoxalinyl, phthalazinyl, imidazo[1,2-a]pyridine, imidazo[2,1-b]thiazolyl, cinnolinyl, triazinyle, benzofurazanyl, azaindolyl, benzimidazolyl, benzothienyl, thienopyridyl, thienopyrimidinyl, pyrrolopyridyl, imidazopyridyl, benzoazaindol, 1,2,4-triazinyl, benzothiazolyl, furanyl, imidazolyl, indolyl, triazolyl, tetrazolyl, indolizinyl, isoxazolyl, isoquinolinyl, isothiazolyl, oxadiazolyl, pyrazinyl, pyridazinyl, pyrazolyl, pyridyl, pyrimidinyl, purinyl, quinazolinyl, quinolinyl, isoquinolyl, 1,3,4-thiadiazolyl, thiazolyl, triazinyl, isothiazolyl, carbazolyl, as well as corresponding groups derived from their fusion or from fusion with the phenyl ring.

The aforementioned “alkyl”, “aryl” and “cycloalkyl” radicals may be substituted with one or more substituents. Among these substituents, mention may be made of the following groups: amino, hydroxy, thio, halogen, alkyl, alkoxy, alkylthio, alkylamino, aryloxy, arylalkoxy, cyano, trifluoromethyl, carboxy or carboxyalkyl.

The “alkoxy” radicals according to the present invention are radicals of formula —O-alkyl, the alkyl group being as defined earlier.

The term of “alkylthio” designates an —S-alkyl group, the alkyl group being as defined above.

The term of “alkylamino” designates an —NH-alkyl, group the alkyl group being as defined above.

Among halogen atoms, mention may more particularly be made of fluorine, chlorine, bromine and iodine atoms.

The term of “aryloxy” designates an —O-aryl group, the aryl group being as defined above.

The term of “arylalkoxy” designates an aryl-alkoxy- group, the aryl and alkoxy groups being as defined above.

The term of “carboxyalkyl” designates an HOOC-alkyl- group, the alkyl group being as defined above. As an example of carboxyalkyl groups, mention may notably be made of carboxymethyl or carboxyethyl.

When an alkyl radical is substituted with an aryl group, one refers to an “arylalkyl” or “aralkyl” radical. The “arylalkyl” or “aralkyl” radicals are aryl-alkyl-radicals, the aryl and alkyl groups being as defined above. Among arylalkyl radicals, mention may notably be made of the benzyl or phenethyl radical.

The expression “pharmaceutically acceptable salts” refers to inorganic and organic, relatively non toxic acid addition salts and to base addition salts of the compounds of the present invention. These salts may be prepared in situ during final isolation and purification of the compounds. In particular, acid addition salts may be prepared by separately reacting the purified compound in its stripped form with an organic or inorganic acid and by isolating the thereby formed salt. Among the examples of acid addition salts, are found hydrobromide, hydrochloride, sulphate, bisulphate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptanate, lactobionate salts, sulphamates, malonates, salicylates, propionates, methylenebis-b-hydroxynaphthoates, gentisic acid, isethionates, di-p-toluoyltartrates, methanesulphonates, ethanesulphonates, benzenesulphonates, p-toluenesulphonates, cyclohexylsulphamates and quinates, laurylsulphonates, and the like (see for example S. M. Berge et al. “Pharmaceutical Salts” J. Pharm. Sci, 66:p. 1-19 (1977)). Acid addition salts may also be prepared by separately reacting the purified compound in its acid form with an organic or inorganic base and by isolating the thereby formed salt. Acid addition salts comprise amine and metal salts. Suitable metal salts comprise sodium, potassium, calcium, barium, zinc, magnesium and aluminium salts. Sodium and potassium salts are preferred. Suitable inorganic base addition salts are prepared from metal bases which comprise sodium hydride, sodium hydroxide, potassium hydroxide, calcium hydroxide, aluminium hydroxide, lithium hydroxide, magnesium hydroxide, zinc hydroxide. Suitable amine base addition salts are prepared from amines which have sufficient alkalinity in order to form a stable salt, and preferably comprise amines which are often used in medicinal chemistry because of their low toxicity and of their acceptability for medical use: ammonia, ethylenediamine, N-methyl-glucamine, lysine, arginine, ornithine, choline, N,N′-dibenzylethylenediamine, chloroprocaine, diethanolamine, procaine, N-benzyl-phenethylamine, diethylamine, piperazine, tris(hydroxymethyl)-aminomethane, tetramethyl-ammonium hydroxide, triethylamine, dibenzylamine, ephenamine, dehydroabietylamine, N-ethylpiperidine, benzylamine, tetra-methylammonium, tetraethylammonium, methylamine, dimethylamine, trimethyl-amine, ethylamine, base amino acids, for example lysine and arginine, and dicyclohexylamine, and the like.

The invention also relates to the tautomeric forms, to enantiomers, diastereoisomers, epimers and organic or mineral salts of the compounds of general formula (I).

Preferably in formula (I), the group R″ is H.

Preferably, in formula (I), the group R₁ is H and R₂ is H or is selected from the group formed by CH(CH₃)₂, NH₂, OH and OMe.

Preferably, when R₁ and R₂ form together with the carbon atom to which they are bound, a group

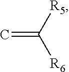

R₅ and R₆ represents a methyl group.

A preferred family of compounds of the invention consists of compounds of formula (I) wherein R represents an aryl group selected from the group formed by phenyl, benzyl, pyridinyl, pyrimidyl, pyrazinyl, triazinyl, pyrazolyl, isoxazolyl, thiazolyl, benzothiazothiazolyl and quinolinyl groups, optionally substituted.

Another preferred family of compounds of the invention consists of compounds of formula (I) wherein R represents a cyclohexyl or piperidinyl group, optionally substituted.

According to a preferred embodiment, the compounds of the invention are compounds of formula (I) as defined above, wherein R is selected from one of the following groups:

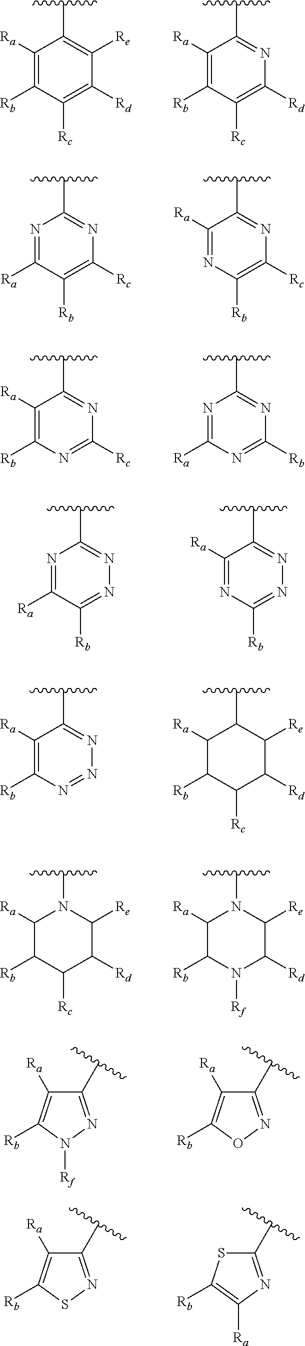

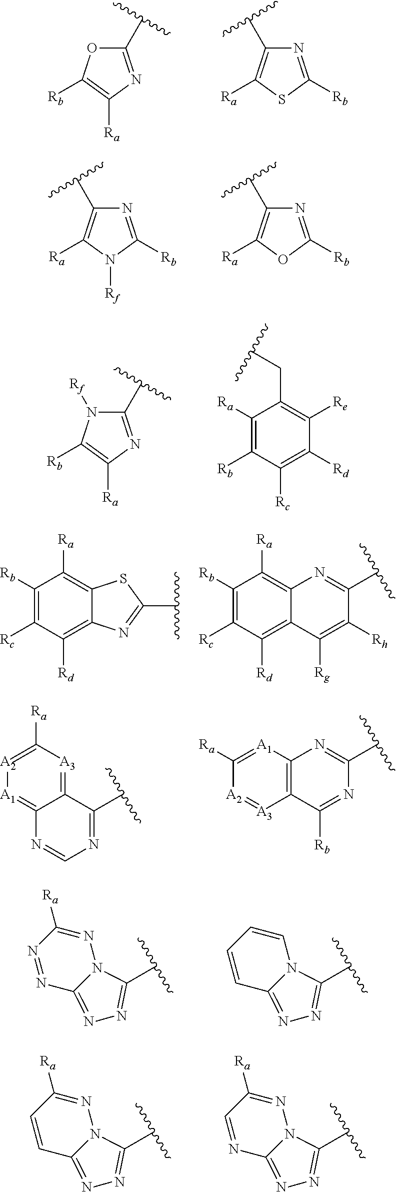

the groups Rₐ, R_{b}, R_{c}, R_{d}, Rₑ, R_{g} and Rₕ being selected independently of each other from the group formed by the following substituents:
a hydrogen atom,
a halogen atom notably Br, Cl or F,
an alkyl group comprising from 1 to 10 carbon atoms and preferably being a methyl group,
said alkyl group being optionally substituted notably with one or more substituents selected from the group formed by the following substituents:
   halogen atoms,
   alkenyl or alkynyl groups comprising from 2 to 10 carbon atoms,
   aryl groups comprising from 6 to 30 carbon atoms,
   CHO, COR_{α}, COOR_{α}, SR_{α}, OR_{α} or NR_{α}R_{β} groups, R_{α} and R_{β} representing independently of each other a hydrogen atom, an alkyl group compromising from 1 to 10 carbon atoms or an aryl group comprising from 6 to 30 carbon atoms,
a —CHO group,
a —CN group,
a phenyl group,
a —SR_{α} or OR_{α} group, R_{α} being as defined above, notably a —OH, —OCH₃, —SH, —SCH₃, —O—CH(CH₃)₂, —O—CH₂—CH₂—CH₃ group,
a —NR_{α}R_{β} group, R_{α} and R_{β} being as defined above, notably an NH₂ group,
a —COOR_{α} group, R_{α} being as defined above, notably —COOMe,
a —CONR_{α}R_{β} group, R_{α} and R_{β} being as defined above, notably —CONH₂,
a —NHCOR_{α} group, R_{α}being as defined above, and
a 2-pyridinyl group,

one of the atoms among A₁, A₂ and A₃ representing N, and the two other atoms among A₁, A₂ and A₃ representing CH,

the R_{f} group being a hydrogen atom, an alkyl group comprising from 1 to 10 carbon atoms or a —COOR_{α} group, R_{α} being as defined above and notably —COOtBu.

“Alkenyl” radicals represent hydrocarbon radicals with a straight or linear chain and comprise one or more ethylenic unsaturations. When they comprise a single double bond they may typically be represented by the formula CₙH₂ₙ, n representing the number of carbon atmos. Among alkenyl radicals, mention may notably be made of allyl or vinyl radicals.

“Alkynyl” radicals represent hydrocarbon radicals with a straight or linear chain and comprise one or more acetylenic unsaturations. When they comprise a single triple bond, they may typically be represented by the formula CₙH₂ₙ₋ₙ, n representing the number of carbon atoms. Among alkynyl radicals, mention may notably made of acetylene.

The preferred groups R are the following:

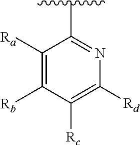

Rₐ, R_{b}, R_{c}, and R_{d}, being selected independently of each other from the group formed by the following substituents: a hydrogen atom, a halogen atom, notably Br, Cl or F, and an alkyl group comprising from 1 to 10 carbon atoms and preferably being a metal group.

Among the latter, mention may notably be made of the following groups:

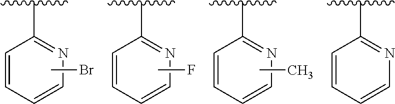

Other preferred groups R are the following:

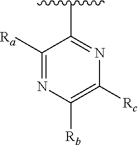

Rₐ, R_{b} and R_{c} being selected independently of each other from the group formed by the following substituents: a hydrogen atom, a halogen atom, notably Br, Cl or F, and an alkyl group comprising from 1 to 10 carbon atoms and preferably being a metal group.

Among the latter, mention may notably be made of the following group:

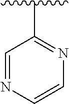

According to another preferred embodiment, the group R fits the following formula:

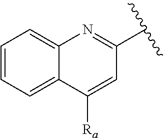

Rₐ being H or an —OR_{α}, group, R_{α} being as defined above and preferably being H or Me.

Among the compounds of the invention, mention may be made of the compounds of the following general formula (I-1):

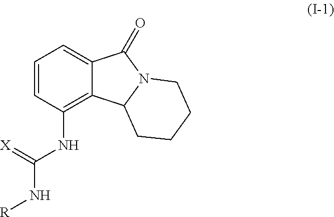

R and X being as defined above.

The compounds of formula (I-1) are compounds of formula (I) in which R₁, R₂, R″ and R₄ are H and A is CX.

Among the compounds of formula (I-1), mention may notably be made of compounds in which R is selected from one of the following groups:

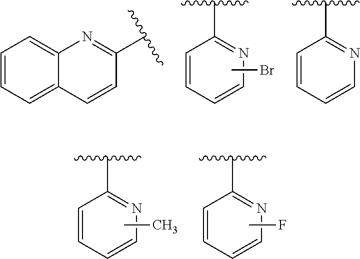

Among the compounds of the invention, mention may also be made of the compounds of the following general formula (I-2):

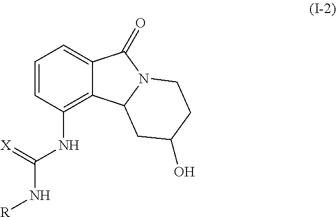

R and X being as defined above.

The compounds of formula (I-2) are compounds of formula (I) in which R₁, R″ and R₄ are H, A is CX and R₂ is OH.

Among the compounds of formula (I-2), mention may notably be made of the compounds in which R

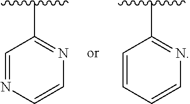

Among the compounds of the invention, mention may also be made of the compounds of the following general formula (I-3):

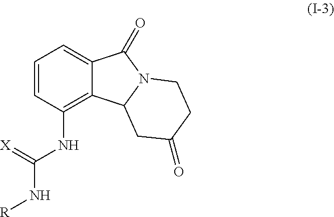

R and X being as defined above.

The compounds of formula (I-3) are compounds of formula (I) in which R″ and R₄ are H, A is CX and R₁ and R₂ form together with the carbon atom to which they are bound, a C═O group.

Among the compounds of formula (I-3), mention may notably be made of the compounds in which R is

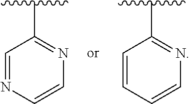

Among the compounds of the invention, mention may also be made of the compounds of the following general formula (I-4):

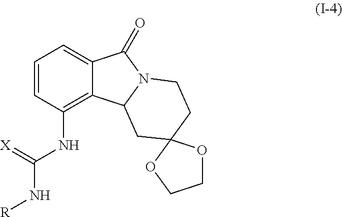

R and X being as defined above.

The compounds of formula (I-4) are compounds of formula (I) in which R″ and R₄ are H, A is CX and R₁ and R₂ form together with the carbon atom to which they are abound, a 1,3-dioxolane group.

Among the compounds of formula (I-4), mention may notably be made of the compounds in which R is

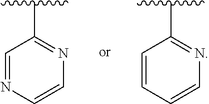

Among the compounds of the invention mention may also be made of the compounds of the following general formula (I-5):

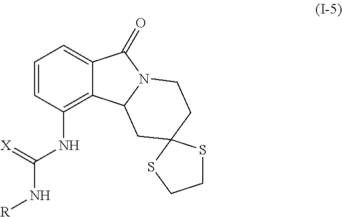

R and X being as defined above.

The compounds of formula (I-5) are compounds of formula (I) in which R″ and R₄ are H, A is CX and R₁ and R₂ form together with the carbon atom to which they are bound, a 1,3-dithiolane group.

Among the compounds of formula (I-5), mention may notably be made of the compounds in which R is

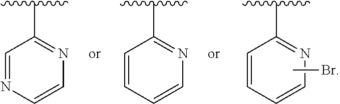

Each family of compounds fitting the formulae (I-1), (I-2), (I-3), (I-4) and (I-5) respectively comprises a sub-family of compounds wherein X is an oxygen atom.

Thus, the present invention relates to the compounds respectively fitting the following formulae (I-1′), (1-2′), (1-3′), (1-4′) and (I-5′):

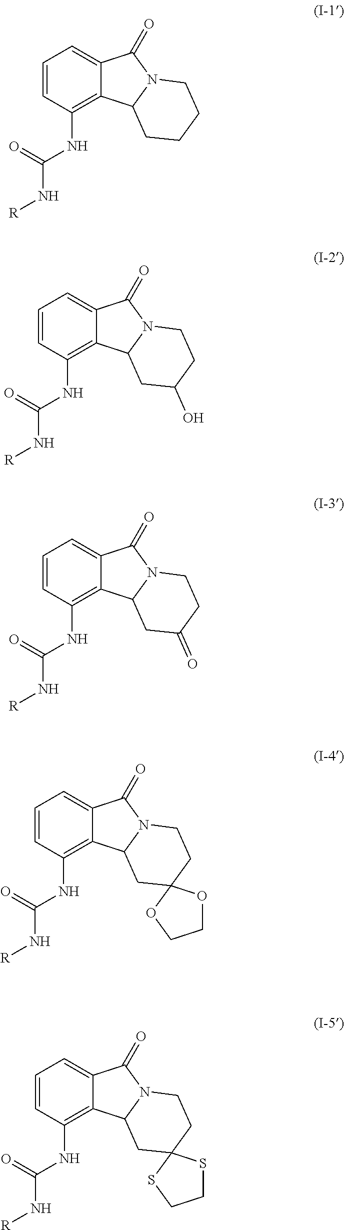

R is as defined above.

Another particular family of compounds of the invention consists of compounds fitting the following general formula (I-1-a):

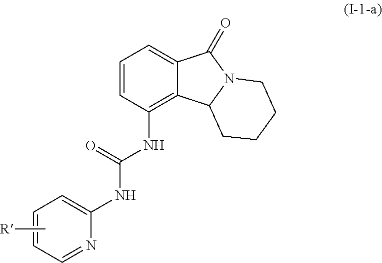

R′ representing a hydrogen atom, a halogen atom such as Br or F or an alkyl group comprising from 1 to 10 carbon atoms, preferably a methyl group.

These compounds are compounds of formula (I-1) as defined above in which X is O and R is a 2-pyridinyl group (optionally substituted with an R′ group).

Among the compounds of formula (I-1-a), mention may notably be made of the following compounds:

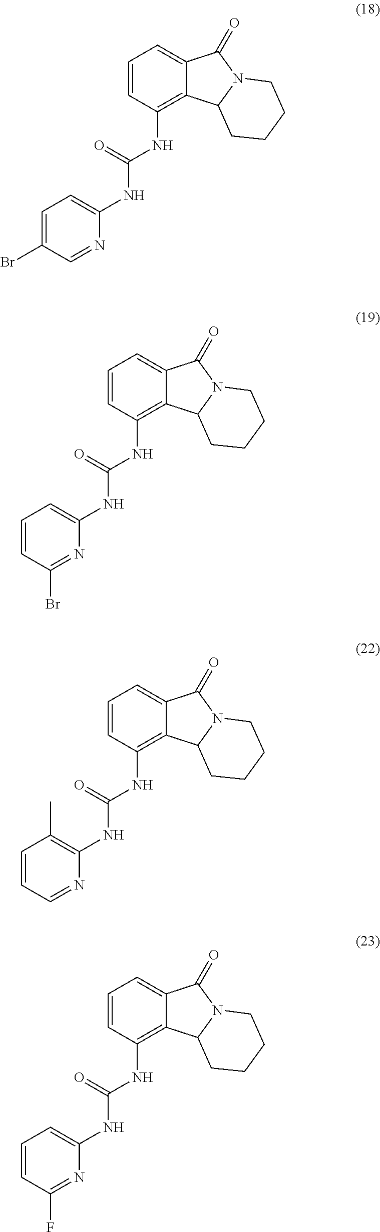

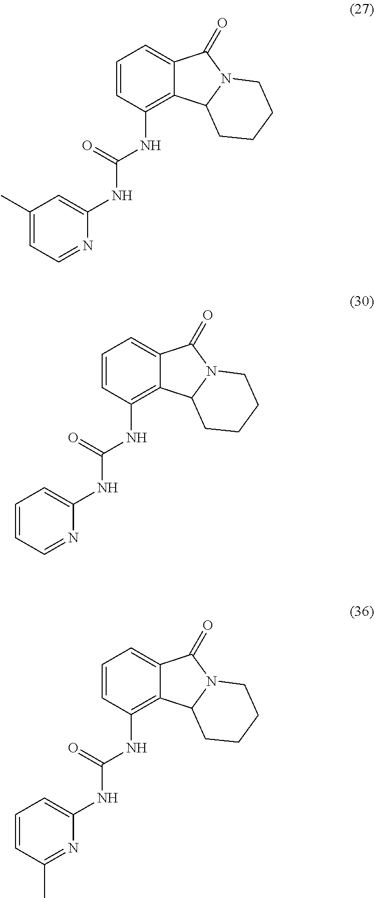

Another particular family of compounds of the invention consists of the compounds fitting the following general formula (I-1-b):

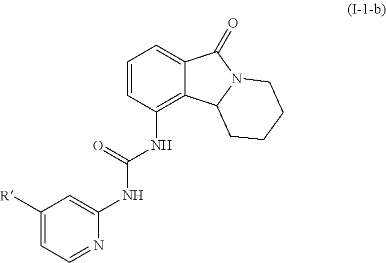

R′ being selected from the group formed by alkyl, aminoalkyl, amide (—CONR_{α}R_{β}, R_{α}and R_{β} being as defined above), alkoxy, notably methoxy or ethoxy, CHO, CH₂CHO and (CH₂)ₙNR_{γ}R_{δ} groups, N being equal to 0 or representing an integer comprised from 1 to 5, preferably N being equal to 0 or 1, and R_{γ} and R_{δ} representing independently of each other a hydrogen atom, an alkyl group comprising from 1 to 10 carbon atoms or an aryl group comprising from 6 to 30 carbon atoms, or R_{γ} and R_{δ} forming with the nitrogen atom to which they are bound, a (hetero)cycloalkyl, said alkyl, aryl and (hetero)cycloalkyl groups being optionally substituted.

These compounds are compounds of formula (I-1) as defined above, in which X is O and R is a 2-pyridinyl group (substituted with a group R′).

Another particular family of compounds of the invention consists of compounds fitting the following general formula (I-5-a):

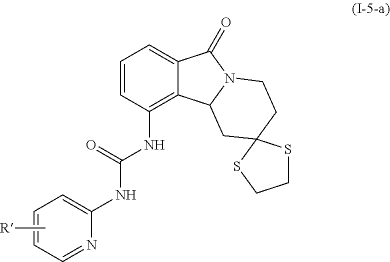

R′ representing a hydrogen atom, a halogen atom such as Br or F or an alkyl group comprising from 1 to 10 carbon atoms, preferably a methyl group.

These compounds are compounds of formula (I-5) as defined above, in which X is O and R is a 2-pyridinyl group (optionally substituted with an R′ group).

Among the compounds of formula (I-5), mention may notably be made of the following compounds:

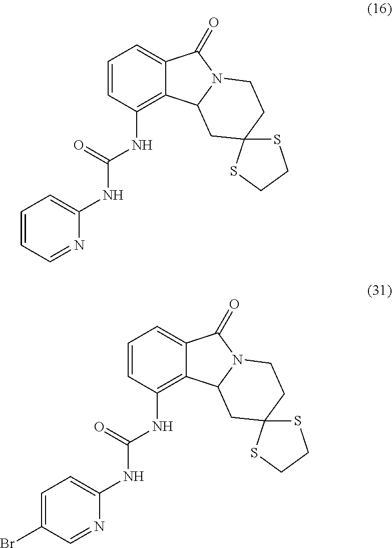

Another particular family of compounds of the invention consists of the compounds fitting the following general formula (I-6):

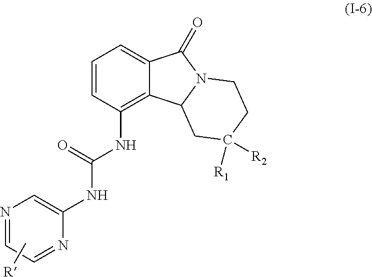

R′, R₁ and R₂ being as defined above,

and preferably R′ representing a hydrogen atom or an alkyl group comprising from 1 to 10 carbon atoms, preferably a methyl group.

The compounds of formula (I-6) are compounds of formula (I) in which R₂ and R₄ are H, A is CO and R is a 2-pyrazinyl group (optionally substituted with an R′ group).

Among the compounds of formula (I-6), mention may notably be made of the following compounds:

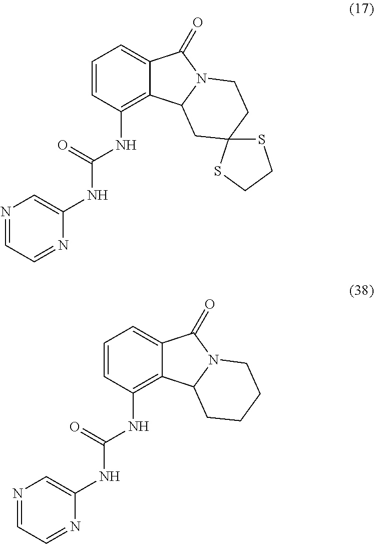

Another particular family of compounds of the invention consists of the compounds fitting the following general formula (I-6-a):

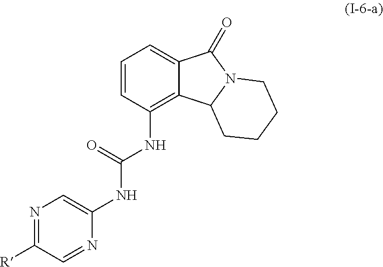

R′ being selected from the group formed by alkyl, aminoalkyl, amide (—CONR_{α}R_{β}, R_{α} and R_{β} being as defined above), alkoxy, notably methoxy or ethoxy, CHO, CH₂CHO and (CH₂)ₙNR_{γ}R_{δ}, n being equal to 0 or representing an integer comprised from 1 to 5, preferably n being equal 0 or 1, and R_{γ} and R_{δ} representing independently of each other a hydrogen atom, and alkyl group comprising from 1 to 10 carbon atoms, or an aryl group comprising from 6 to 30 carbon atoms, or R_{γ} and R_{δ} forming with the nitrogen atom to which they are bound, a heterocycloalkyl, said alkyl, aryl and heterocycloalkyl groups being optionally substituted.

The compounds of formula (I-6-a) are compounds of formula (I-6) wherein R₁ and R₂ are H.

Another particular family of compounds of the invention consists of compounds fitting the following general formula (I-7):

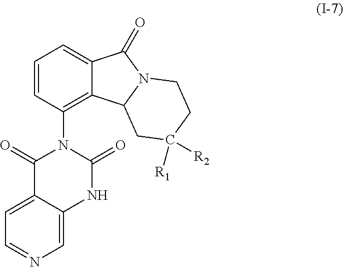

R₁ and R₂ being as defined above.

The compounds of formula (I-7) are compounds of formula (I) wherein A is a —CO— group and R and R₄ form together with the nitrogen atoms to which they are attached, a ring of formula (II) as defined above in which A₁, A₁ and A₄ are H and A₂ is N.

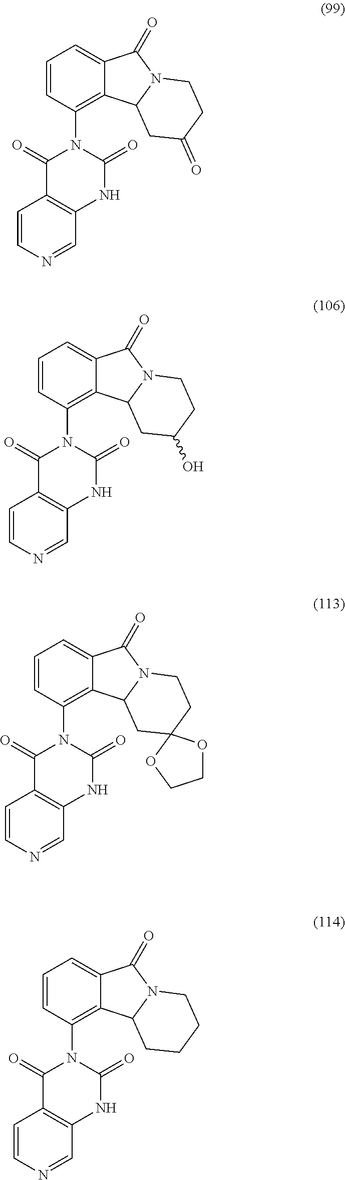

The present invention also relates to the compounds of the following formula (I-8):

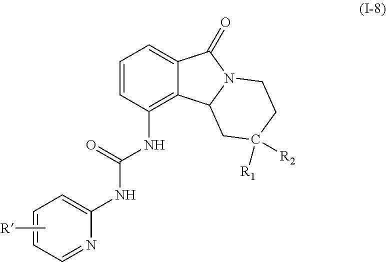

R′, R₁ and R₂ are as defined above.

The present invention also relates to a pharmaceutical composition comprising a compound of formula (I) as defined above, or any compound as mentioned above, in combination with a pharmaceutically acceptable carrier.

The present invention therefore relates to a compound as defined above of formula (I) for its use as a drug.

The pharmaceutical compounds according to the invention may have presentation forms intended for administration via a parenteral, oral, rectal, permucosal or percutaneous route.

The pharmaceutical compositions including these compounds of general formula (I) will therefore appear as solutes or injectable suspensions or multidose flasks, as naked or coated tablets, dragees, capsules, gelatin capsules, pills, cachets, powders, suppositories or rectal capsules, solutions or suspensions, for percutaneous use in a polar solvent, for permucosal use.

The excipients which are suitable for such administrations are derivatives of cellulose or microcrystalline cellulose, earth alkaline carbonates, magnesium phosphate, starches, modified starches, lactose for solid forms.

For rectal use, cocoa butter or polyethylene glycol steroids are the preferred excipients.

For parenteral use, water, aqueous solutes, saline, isotonic solutes are the most conveniently used carriers.

The dosage may vary within large limits (0.5 mg to 1000 mg) depending on the therapeutic indication and on the administration route, as well as on the age and weight of the subject.

The present invention relates to compounds of the invention as defined above, for their use as an inhibitor of CDK1, CDK5 and/or GSK-3 kinases.

Thus, the present invention relates to compounds of the invention as defined above, for their use within the scope of the treatment or prevention of diseases related to deregulation of CDK1, CDK5 and/or GSK-3 kinases.

More particularly, said diseases are selected from the group consisting of cancers, Alzheimer's disease, Parkinson's disease, brain traumas, strokes, renal polycystosis, amyotrophic lateral sclerosis, viral infections, autoimmune diseases, neurodegenerative disorders, psoriasis, asthma, atypical dermatitises and glomerulonephrites.

The present invention also relates to the use of the compounds of the invention as defined above, for the preparation of a drug intended for treating or preventing diseases related to deregulation of the CDK1, CDK5 and/or GSK-3 kinases, and more particularly to the treatment and prevention of the aforementioned diseases.

The present invention also relates to the method of preparation of the compounds of general formula (I) as defined above, in which R₄ and R″ are H and A is a CO group, and R₁ and R₂ are H or form together with the carbon atom to which they are attached, a group:

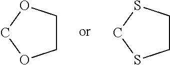

said method comprising:

a reaction step of the amine of the following formula:

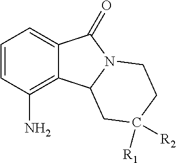

with an isocyanate of formula RNCO, notably in the presence of dioxane at 100° C. for 24 hours, R being as defined above for formula (I).

The present invention also relates to the method of preparation of the compounds of general formula (I) as defined above, in which R₄ and R″ are H and A is a CS group, and R₁ and R₂ are H or form together with the carbon atom to which they are attached, a group

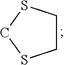

said method comprising:

a reaction step of the isothiocyanate of the following formula:

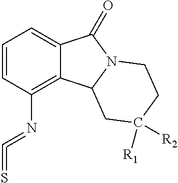

with an amine of formula RNH₂, notably in the presence of dioxane at 100° C. for 24 hours, R being as defined above for formula (I).

The present invention also relates to the method of preparation of the compounds of general formula (I) as defined above, in which R₄ and R″ are H and A is a CO group, and R₁ and R₂ are H or form together with the carbon atom to which they are attached a group

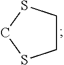

said method compromising:

a reaction step of the thiourea of the following formula:

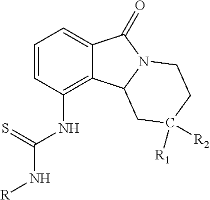

R being as defined above for formula (I),

in the presence of a CH₃CN/water mixture and of HgO at room temperature for a duration comprised from 24 to 40 hours.

The present invention also relates to the method of preparation of the compounds of general formula (I) as defined above, in which R₄ and R″ are H and A is a CO group, and R₁ and R₂ are H or form together with the carbon atom to which they are attached a group

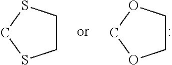

said method comprising:

a reaction step of the amine of the following formula:

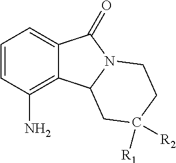

with an amine of formula RNH₂ and of triphosgen, notably in the presence of iPr₂NEt in dichloromethane at room temperature for one hour, R being as defined above for formula (I).

The present invention also relates to the method of preparation of the compounds of general formula (I) as defined above, in which R₁ and R₂ form together with the carbon atom to which they are attached, a C═O group; said method comprising:

a reaction step of the acetal of the following formula:

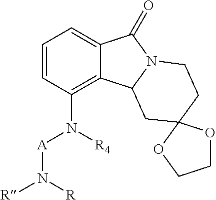

A, R, R₂ and R₄ being as defined above in formula (I),

with acetone and hydrochloric acid, the whole being notably refluxed for three hours.

The present invention also relates to the method of preparation of the compounds of general formula (I) as defined above, in which R₁ is H and R₂ is OH,

said method comprising:

a step for reducing the ketone of the following formula:

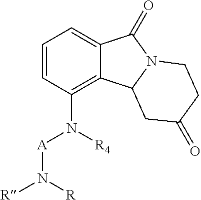

A, R, R″ and R₄ being as defined above for formula (I),

preferably with NaBH₄ in the presence of THF and methanol, the whole being notably stirred for two hours at a temperature comprised from 0° C. to 5° C.

The present invention also relates to the method of preparation of the compounds of general formula (I-7) as defined above, in which R₁ and R₂ are H or R₁ and R₂ form together with the carbon atom to which they attached, a group

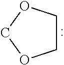

said method comprising:

a reaction step of the amine of the following formula:

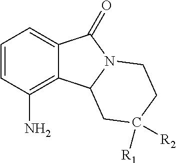

R₁ and R₂ being as defined above, with an isocyanate of formula (a2)

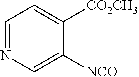

notably in toluene, said amine for example being in pyridine, the reaction medium being stirred with reflux for 24 hours.

The present invention also relates to the intermediate compounds (used for the preparation of the components of formula (I) according to the invention) fitting one of the following formulae:

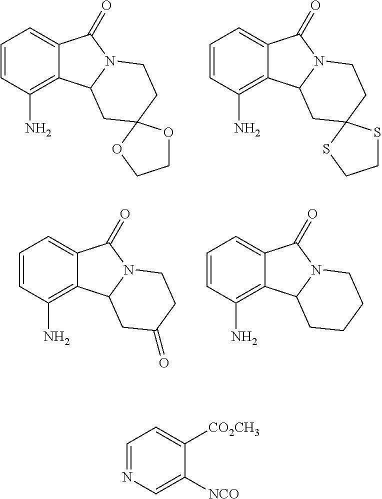

### EXPERIMENTAL PART

### Preparation of the Compounds of the Invention

1. Preparation of the Synthesis Intermediates

The methods of preparation of the compounds of the invention are carried out from synthesis intermediates (1)-(15), prepared according to the following scheme:

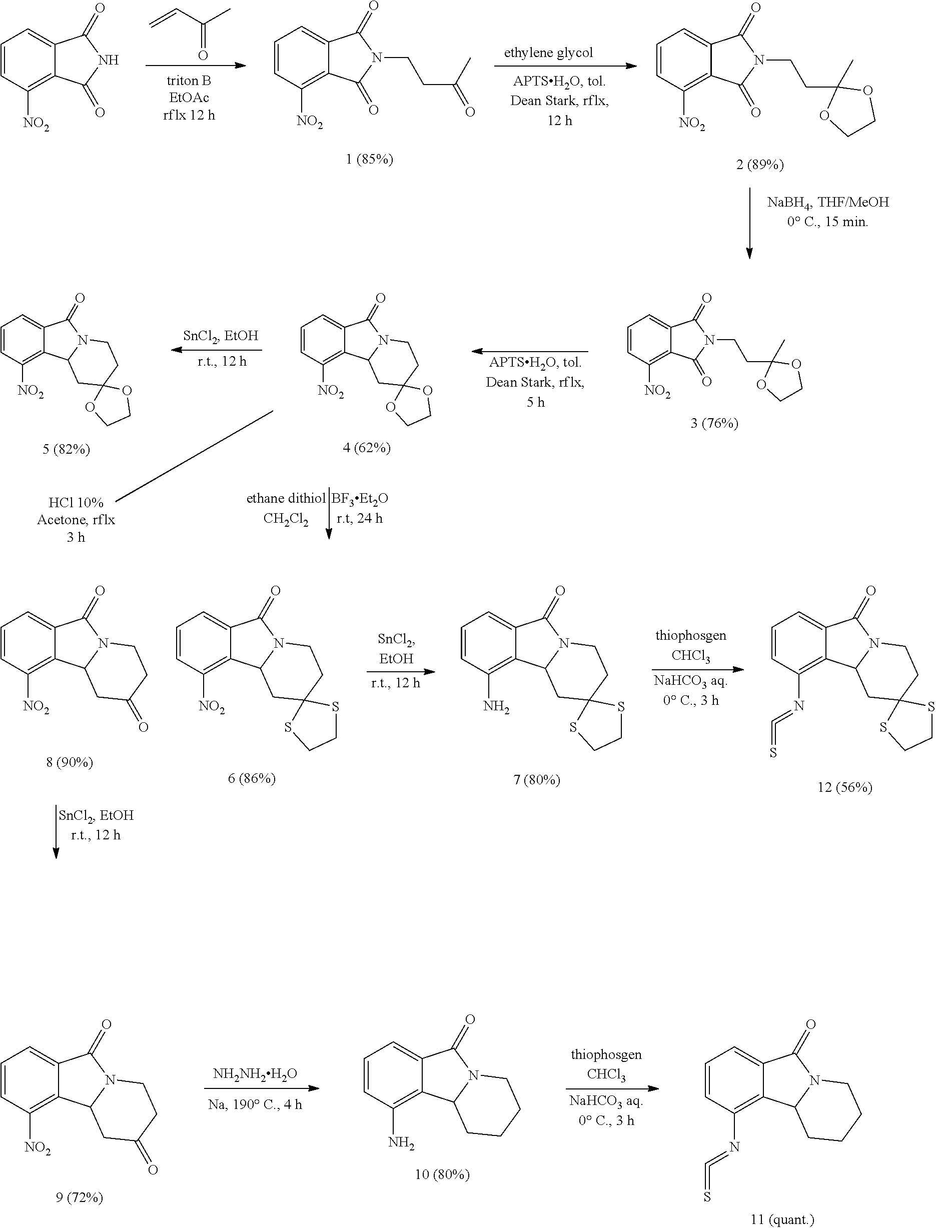

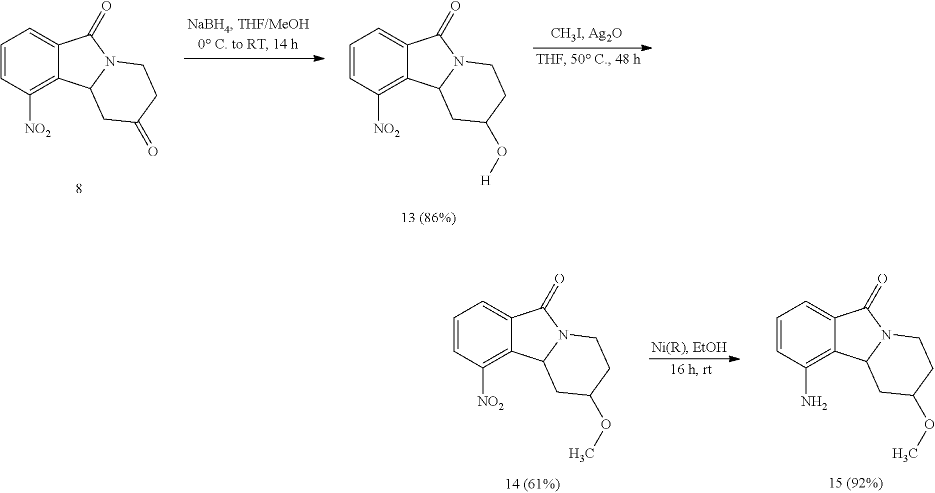

### 4-Nitro-2-(3-oxobutyl)isoindoline-1,3-done (1)

To a solution containing 3-nitrophthalimide (5.00 g; 26 mmol) in ethyl acetate (30 mL), methyl vinyl ketone (2.8 mL; 33 mmol; 1.25 equ.) is added dropwise. After a few minutes of stirring, 760 μL of triton B (Benzyltrimethylammonium hydroxide, 40% in water) are added. The reaction medium is refluxed for 12 hours. After cooling, the solvents are removed under reduced pressure and the solid residue is recrystallized from absolute ethanol in order to obtain the compound 1 as a yellow solid with a yield of 85%. MP: 122° C.; IR (ATR-Ge v cm⁻¹): 1715 (C═O ketone), 1538 (C═C arom), 1373 (N═O₂), 1127 (C—C); ¹H NMR (CDCl₃; 250 MHz) δ8.19-8.07 (m, 2H), 7.93 (dd, J=7.1 Hz, J=8.5 Hz, 1H), 3.98 (t, J=7.3 Hz, 2H), 2.91 (t, J=7.3 Hz, 2H), 2.20 (s, 3H); ¹³C NMR (CDCl₃; 63 MHz) δ205.7 (Cq), 165.6 (Cq), 162.9 (Cq), 145.2 (Cq), 135.6 (CH), 134.2 (Cq), 128.7 (CH), 127.2 (CH), 123.9 (Cq), 41.2 (CH₂), 33.8 (CH₂), 30.1 (CH₃). MS (IS) m/z: 263.0 [M+H]⁺

### 2-(2-(2-Methyl-1,3-dioxolan-2-yl)ethyl)-4-nitroisoindoline-1,3-dione (2)

To a solution containing the compound 1 (5.0 g, 19.0 mmol) in toluene (60 mL) are successfully added ethylene glycol (2.0 mL, 38.0 mmol, 2.0 equ.) and APTS.H₂O (21 mg; 0.1 mmol, cat.). The reaction medium is refluxed for one night by using a Dean-Stark apparatus. The cooled reaction mixture is washed with a solution saturated with NaHCO₃ (20 mL) and then with a solution saturated with NaCl (20 mL). The organic extract is dried on MgSO₄ and then the solvents are evaporated. The solid is recrystallized from ethanol in order to obtain the compound 2 as a yellow solid with a yield of 89%. MP: 90° C.; IR (ATR-Ge v cm⁻¹): 1711 (C═O amide), 1542 (C═C arom), 1366 (N═O₂), 1158 (C—O); ¹H NMR (CDCl₃; 250 MHz) δ 8.17-8.05 (m, 2H), 7.90 (dd, J=7.4 Hz, J=8.2 Hz, 1H), 4.00-3.90 (m, 4H), 3.86 (t, J=7.0 Hz, 2H), 2.10 (t, J=7.0 Hz, 2H), 1.36 (s, 3H); ¹³C NMR (CDCl₃; 63 MHz) δ 165.9 (Cq), 163.0 (Cq), 145.2 (Cq), 135.3 (CH), 134.4 (Cq), 128.5 (CH), 127.0 (CH), 124.1 (Cq), 108.8 (Cq), 64.8 (2×CH₂), 36.1 (CH₂), 34.3 (CH₂), 23.9 (CH₃). MS (IS) m/z: 307.5 [M+H]⁺, 324.0 [M+NH₄]⁺

### 3-Hydroxy-2-(2-(2-methyl-1,3-dioxolan-2-yl)ethyl)-4-nitroisoindolin-1-one (3)

A solution containing the compound 2 (2.0 g, 6.5 mmol) dissolved in a THF/MeOH mixture 1-2 (15/30 mL) is placed at −20° C. under stirring. 700 mg of NaBH₄ (19.5 mmol, 3.0 equ.) are added portionwise and then the reaction medium is stirred at this temperature for 15 minutes. The temperature is raised again to 0° C. and then an aqueous solution of NaOH 1M (40 mL) is introduced. The mixture is brought to room temperature, the volatiles are evaporated under reduced pressure. The residual aqueous phase is extracted with dichloromethane (3×20 mL). The collected organic extracts are dried on MgSO₄, filtered and evaporated under reduced pressure. Flash chromatography on silica gel (dichloromethane/methanol 99/1) gives the product 3 as a white solid with a yield of 76%. MP: 130° C. IR (ATR-Ge v cm⁻¹): 3293 (O—H), 1687 (C═O amide), 1540 (C═C arom), 1348 (N═O₂), 1054 (C—O); ¹H NMR (CDCl₃; 250 MHz) δ 8.32 (dd, J=0.9 Hz, J=8.2 Hz, 1H), 8.09 (dd, J=0.7 Hz, J=7.4 Hz, 1H), 7.77-7.67 (m, 1H), 6.46 (d, J=5.4 Hz, 1H), 4.23 (d, J=5.4 Hz, 1H), 3.95 (m, 4H), 3.86 (m, 1H), 3.75-3.59 (m, 1H), 2.21-2.05 (m, 2H), 1.39 (s, 3H); ¹³C NMR (CDCl₃; 63 MHz) δ 164.8 (Cq), 143.8 (Cq), 138.9 (Cq), 135.3 (Cq), 131.5 (CH), 129.6 (CH), 127.2 (CH), 109.1 (Cq), 81.4 (CH), 64.8 (CH₂), 64.8 (CH₂), 36.9 (CH₂), 35.8 (CH₂), 24.0 (CH₃). MS (IS) m/z: 309.0 [M+H]⁺, 326.0 [M+NH₄]⁺

### 10′-Nitro-3′,4′-dihydro-1′H-spiro[[1,3]dioxolane-2,2′-pyrido[2,1-a]isoindol]-6′(10b′H)-one (4)

In a 100 mL flask, surmounted with a Dean-Stark apparatus, a solution containing beta-hydroxylactam 3 (500 mg, 1.62 mmol) and APTS.H₂O (156 mg, 0.8 mmol, 0.5 equ.) in toluene (50 mL) is refluxed with heating for 6 hours. After cooling, a solution saturated with NaHCO₃ (45 mL) is added. After extraction, the aqueous phase is taken up with ethyl acetate (20 mL). The collected organic phases are dried on MgSO₄ and the solvents are evaporated under reduced pressure. The obtained residue is purified by silica gel chromatography (dichloromethane/methanol 99.5/0.5) in order to obtain the product 4 as a yellow solid with a yield of 62%. MP: 206° C.; IR (ATR-Ge v cm⁻¹): 1685 (C═O amide), 1539 (C═C arom), 1360 (N═O₂), 1142 (C—O), 1028 (C—C); ¹H NMR (CDCl₃, 250 MHz) δ 8.37 (d, J=8.1 Hz, 1H), 8.19 (d, J=7.4 Hz, 1H), 7.70 (t, J=7.8 Hz, 1H), 5.23 (dd, J=3.3 Hz, J=11.7 Hz, 1H), 4.55 (dd, J=4.3 Hz, J=13.4 Hz, 1H), 4.31-3.96 (m, 4H), 3.33 (td, J=4.2 Hz, J=13.0 Hz, 1H), 2.78-2.61 (m, 1H), 1.95-1.65 (m, 2H), 1.17 (t, J=12.2 Hz, 1H); ¹³C NMR (CDCl₃; 63 MHz) δ 163.8 (Cq), 143.7 (Cq), 140.0 (Cq), 136.0 (Cq), 130.2 (CH), 130.0 (CH), 127.1 (CH), 107.30 (Cq), 65.0 (CH₂), 64.9 (CH₂), 58.4 (CH), 38.1 (CH₂), 37.0 (CH₂), 34.3 (CH₂); MS (IS) m/z: 291.5 [M+H]⁺.

### 10′-Amino-3′,4′-dihydro-1′H-spiro[[1,3]dioxolane-2,2′-pyrido[2,1-a]isoindol]-6′(10b′H)-one (5)

To a solution of the compound 4 (1.0 g, 3.4 mmol) in EtOH (20 mL) is added anhydrous SnCl₂ (9.5 g, 51.0 mmol, 15.0 equ.) at room temperature. The mixture is stirred for one night and then the ethanol is evaporated under reduced pressure without heating. The solid residue is placed at 0° C., an aqueous solution of NaOH (2M) is added up to a pH=7. The aqueous phases are extracted with dichloromethane (3×20 mL). The collected organic phases are dried on MgSO₄ and then the solvent is evaporated under reduced pressure. The solid residue is purified by silica gel chromatography (dichloromethane/methanol 99/1) in order to obtain the compound 5 as a white solid with a yield of 82%. MP: 76° C.; IR (ATR-Ge v cm⁻¹): 1710 (C═O amide), 1542 (C═C arom), 1366 (C—N), 1143 (C—O), 1028 (C—C); ¹H NMR (CDCl₃; 250 MHz) δ 7.36-7.24 (m, 2H), 6.81 (dd, J=1.8 Hz, 7.0 Hz, 1H), 4.61-4.41 (m, 2H), 4.14-3.99 (m, 4H), 3.74 (s, 2H), 3.23 (td, J=3.9 Hz, J=13.1 Hz, 1H), 2.45 (m, 1H), 1.82 (m, 1H), 1.67 (td, J=5.7 Hz, 13.0 Hz, 1H), 1.38 (t, J=12.5 Hz, 1H); ¹³C NMR (CDCl₃; 63 MHz) δ 166.8 (Cq), 141.2 (Cq), 133.48 (Cq), 129.6 (CH), 129.5 (Cq), 118.4 (CH), 114.5 (CH), 107.7 (Cq), 65.0 (CH₂), 64.8 (CH₂), 55.8 (CH), 39.1 (CH₂), 36.6 (CH₂), 34.1 (CH₂); MS (IS) m/z: 261 [M+H]⁺

### 10′-Nitro-3′,4′-dihydro-1′H-spiro[[1,3]dithiolane-2,2′-pyrido[2,1-a]isoindol]-6′(10b′H)-one (6)

To a solution of the compound 4 (1.0 g, 3.4 mmol) in dichloromethane (40 mL), are slowly added with precaution, ethane dithiol (1.24 mL, 17 mmol, 5 equ.), BF₃.Et₂O (2.15 mL, 17.0 mmol, 5.0 equ.). After stirring for 24 hours at room temperature, CH₂Cl₂ (40 mL) and an aqueous solution of NaOH 1M (40 mL) are added. After extraction, the organic phase is dried on MgSO₄ and then the solvents are evaporated under reduced pressure. The residue is purified by flash chromatography on silica gel (ethyl acetate/petroleum ether 60/40) in order to obtain the product 6 as a beige powder with a yield of 86%. MP: 185° C.; IR (ATR-Ge v cm⁻¹): 1689 (C═O amide), 1524 (C═C arom), 1416 (C—N), 1345 (N═O₂), 1079 (C—C); ¹H NMR (CDCl₃; 250 MHz) δ 8.36 (dd, J=0.9 Hz, 8.2 Hz, 1H), 8.18 (dd, J=0.6 Hz, J=7.5 Hz, 1H), 7.69 (t, J=7.8 Hz, 1H), 5.25 (dd, J=3.1 Hz, J=11.1 Hz, 1H), 4.57 (m, 1H), 3.56-3.36 (m, 4H), 3.36-3.19 (m, 1H), 3.10-2.97 (m, 1H), 2.31-1.97 (m, 2H), 1.51 (dd, J=11.2 Hz, J=12.9 Hz, 1H); ¹³C NMR (CDCl₃; 63 MHz) δ 163.7 (Cq), 143.8 (Cq), 139.6 (Cq), 135.9 (Cq), 130.2 (CH), 130.1 (CH), 127.1 (CH), 65.5 (Cq), 59.5 (CH), 45.4 (CH₂), 40.6 (CH₂), 39.4 (CH₂), 39.3 (CH₂), 38.7 (CH₂); MS (IS) m/z: 323.0 [M+H]⁺, 340.0 [M+NH₄]⁺

### 10′-Amino-3′,4′-dihydro-1′H-spiro[[1,3]dithiolane-2,7-pyrido[2,1-a]isoindol]-6′(10b′H)one (7)

To a solution containing the compound 6 (800 mg, 2.4 mmol) of the compound 6 in l'EtOH (20 mL) are added 6.8 g de SnCl₂ (36.0 mmol, 15.0 equ.). The mixture is stirred for one night and then the ethanol is evaporated under reduced pressure without heating. The residue is cooled to 0° C. and then neutralized by successive additions of an aqueous solution of NaOH 2M. The aqueous phase is extracted with dichloromethane (3×20 mL), the collected organic phases are then dried on MgSO₄, filtered and evaporated under reduced pressure. The residue is purified by chromatography on silica gel (dichloromethane/methanol 99/1) in order to obtain the compound 7 as a yellow solid with a yield of 80%. MP: 205° C.; IR (ATR-Ge v cm⁻¹): 3236 (NH₂), 1675 (C═O amide), 1487 (C═C arom), 1287 (C—N), 1003 (C—C); ¹H NMR (CDCl₃; 400 MHz) δ 7.37-7.17 (m, 2H), 6.80 (dd, J=1.3 Hz, 7.2 Hz, 1H), 4.60-4.41 (m, 2H), 3.81 (s, 2H), 3.50-3.31 (m, 4H), 3.23 (td, J=3.2 Hz, J=13.3 Hz, 1H), 2.92-2.78 (m, 1H), 2.24-2.13 (m, 1H), 2.02 (td, J=5.1 Hz, J=12.9 Hz, 1H), 1.77-1.63 (m, 1H); ¹³C NMR (CDCl₃; 101 MHz) δ 166.7 (Cq), 141.4 (Cq), 133.3 (Cq), 129.7 (CH), 129.0 (Cq), 118.4 (CH), 114.3 (CH), 65.7 (Cq), 57.1 (CH), 45.7 (CH₂), 41.6 (CH₂), 39.66 (CH₂), 38.7 (CH₂), 38.4 (CH₂); MS (IS) m/z: 293.0 [M+H]⁺.

### 10-Nitro-1,3,4,10b-tetrahydropyrido[2,1-a]isoindole-2,6-done (8)

To a solution of the compound 4 (2.0 g, 6.91 mmol) in the acetone (30 mL) is added an aqueous solution of 10% hydrochloric acid (20 mL). The reaction medium is refluxed for 3 hours and then concentrated to 50% by evaporation under reduced pressure. After cooling in an ice water bath, the precipitate is filtered and then washed with water (10 mL) and dried under reduced pressure in order to obtain the compound 8 as a yellow solid with a yield of 90%. MP: 215° C.; IR (ATR-Ge v cm⁻¹): 1692 (C═O ketone), 1529 (C═C arom), 1342 (NO₂), 1298 (C—N); ¹H NMR (CDCl₃; 400 MHz) δ 8.44 (d, J=8.0 Hz, 1H), 8.25 (d, J=7.4 Hz, 1H), 7.78 (t, J=7.8 Hz, 1H), 5.39 (dd, J=4.0 Hz, 11.6 Hz, 1H), 4.82-4.76 (m, 1H), 3.55-3.38 (m, 2H), 2.72-2.52 (m, 2H), 2.08 (dd, J=11.7 Hz, 14.5 Hz, 1H); ¹³C NMR (CDCl₃; 101 MHz) δ 204.4 (Cq), 164.1 (Cq), 143.6 (Cq), 139.2 (Cq), 135.4 (Cq), 130.7 (CH), 130.6 (CH), 127.7 (CH), 58.7 (CH), 44.5 (CH₂), 39.5 (CH₂), 37.3 (CH₂); MS (IS) m/z: 293.5 [M+H]⁺

### 10-Amino-1,3,4,10b-tetrahydropyrido[2,1-a]isoindole-2,6-dione (9)

To a solution containing the compound 8 (3.0 g, 12.2 mmol) dissolved in EtOH (80 mL) are added portionwise 34.0 g of SnCl₂ (180 mmol, 15.0 equ.) and then 46 mL of an aqueous solution of 12N hydrochloric acid. The reaction medium is stirred for one night at room temperature and then the ethanol is evaporated under reduced pressure at a low temperature. The residue is placed at 0° C. and then neutralized with an aqueous solution of NaOH 2M. The aqueous phase is extracted with dichloromethane (3×50 mL) and then the collected organic phases are dried on MgSO₄, filtered and evaporated under reduced pressure. The residue is purified by flash chromatography on silica gel (dichloromethane/methanol 99/1) in order to obtain the compound 9 as a white solid with a yield of 72%. IR (ATR-Ge v cm⁻¹): ¹H NMR (CDCl₃, 400 MHz) δ 7.34-7.27 (m, 2H), 6.85 (dd, J=3.9 Hz, 4.9 Hz, 1H), 4.79-4.73 (m, 1H), 4.66 (dd, J=3.9 Hz, 11.9 Hz, 1H), 3.90 (s, 2H), 3.40-3.32 (m, 1H), 3.21-3.16 (m, 1H), 2.65-2.47 (m, 2H), 2.21 (dd, J=12.2 Hz, 13.8 Hz, 1H) ¹³C NMR (CDCl₃, 101 MHz) δ 206.4 (Cq), 167.0 (Cq), 141.4 (Cq), 132.7 (Cq), 130.2 (CH), 128.5 (Cq), 118.9 (CH), 114.4 (CH), 56.8 (CH), 45.0 (CH₂), 40.0 (CH₂), 37.5 (CH₂); MS (IS) m/z:

### 10-Amino-1,2,3,4-tetrahydropyrido[2,1-a]isoindol-6(10bH)-one (10)

In a flask containing 9 mL of ethylene glycol are added with stirring and with precaution 160 mg of sodium (6.93 mmol, 3.0 equ.) at room temperature and then 500 mg of the ketone 9 (2.31 mmol) and finally 0.45 mL of hydrazine monohydrate (9.24 mmol, 4 equ.). The reaction medium is heated for 4 hours at 190° C. After complete disappearance of the initial product, the mixture is cooled and dichloromethane (50 mL) is added. The organic phase is washed with an aqueous solution of NaOH 1M, dried on MgSO₄, filtered and then the solvents are evaporated. The raw product is purified by chromatography on silica gel (dichloromethane/methanol 99.5/0.5) in order to obtain the compound 10 as a yellow solid with a yield of 80%. IR (ATR-Ge v cm⁻¹): ¹H NMR (CDCl₃; 250 MHz) δ 7.35-7.17 (m, 2H), 6.80 (dd, J=1.4 Hz, 7.3 Hz, 1H), 4.49 (dd, J=4.9 Hz, J=13.3 Hz, 1H), 4.23 (dd, J=3.6 Hz, J=11.7 Hz, 1H), 3.79 (s, 2H), 2.96 (td, J=3.5 Hz, J=12.9 Hz, 1H), 2.50 (dd, J=3.2 Hz, J=13.4 Hz, 1H), 1.99 (m, 1H), 1.88-1.74 (m, 1H), 1.74-1.52 (m, 1H), 1.52-1.29 (m, 1H), 1.11 (m, 1H); ¹³C NMR (CDCl₃; 63 MHz) δ 166.6 (Cq), 141.3 (Cq), 133.6 (Cq), 130.2 (Cq), 129.3 (CH), 118.1 (CH), 114.2 (CH), 57.9 (CH), 39.9 (CH₂), 30.4 (CH₂), 25.5 (CH₂), 23.7 (CH₂); MS (IS) m/z: 203.0 [M+H]⁺.

### 10-Isothiocyanato-1,2,3,4-tetrahydropyrido[2,1-a]isoindol-6(10bH)-one (11)

To a solution containing the amine 10 (100 mg, 0.49 mmol) in chloroform (20 mL) is added a solution of 289 mg of NaHCO₃ in 20 mL of water. The mixture is cooled to 0° C. and then 50 μL of thiophosgen (0.64 mmol, 1.3 equ.) are added. The solution is stirred for 3 hours. After extraction with chloroform (3×20 mL), the collected organic phases are dried on MgSO₄, filtered and concentrated under reduced pressure. The residue is purified by silica gel chromatography (dichloromethane) in order to obtain the product 11 as an oil with quantitative yield. IR (ATR-Ge v cm⁻¹): 2056 (N═C═S), 1687 (C═O amide), 1591-1475-1417 (C═C arom), 1284 (C—N); ¹H NMR (CDCl₃; 400 MHz) δ 7.77 (d, J=7.5 Hz, 1H), 7.46 (t, J=7.7 Hz, 1H), 7.37 (d, J=7.9 Hz, 1H), 4.50 (dd, J=5.0 Hz, 13.3 Hz, 1H), 4.40 (dd, J=3.6 Hz, 12.4 Hz, 1H), 3.00 (td, J=3.6 Hz, 12.4 Hz, 1H), 2.64 (dd, J=3.1 Hz, 12.9 Hz, 1H), 2.05 (d, J=13.7 Hz, 1H), 1.90-1.80 (m, 1H), 1.80-1.64 (m, 1H), 1.49-1.34 (m, 1H), 1.17-1.07 (m, 1H); ¹³C NMR (CDCl₃; 101 MHz) δ 164.9 (Cq), 140.8 (Cq), 138.2 (Cq), 134.8 (Cq), 129.7 (CH), 128.7 (CH), 126.5 (Cq), 122.8 (CH), 58.2 (CH), 40.0 (CH₂), 30.8 (CH₂), 25.3 (CH₂), 23.6 (CH₂).

### 10′-Isothiocyanato-3′,4′-dihydro-1H-spiro[[1,3]dithiolane-2,2′-pyrido[2,1-a]isoindol]-6′(10b′H)-one (12)

A solution containing the amine 7 (900 mg, 3.0 mmol) in chloroform (120 mL) and a solution of 1.78 g of NaHCO₃ in 115 mL of water is cooled to 0° C. The thiophosgen (0.3 mL, 3.9 mmol) is added dropwise and then the solution is stirred for 3 hours. After extraction with chloroform (3×20 mL), the collected organic phases are dried on MgSO₄, filtered and concentrated under reduced pressure. The residue is purified by silica gel chromatography (dichloromethane) in order to obtain the product 12 as a white powder with a yield of 56%. MP: 150° C.; IR (ATR-Ge v cm⁻¹): 2085 (N═C═S), 1690 (C═O amide), 1592-1474-1417 (C═C arom), 1258 (C—N); ¹H NMR (CDCl₃; 250 MHz) δ 7.65 (dd, J=0.8 Hz, J=7.5 Hz, 1H), 7.38 (t, J=7.7 Hz, 1H), 7.26 (dd, J=1.0 Hz, J=7.9 Hz, 1H), 4.63 (dd, J=3.4 Hz, J=11.6 Hz, 1H), 4.43 (m, 1H), 3.49-3.27 (m, 4H), 3.13 (td, J=3.4 Hz, J=13.2 Hz, 1H), 2.86 (m, 1H), 2.22-2.07 (m, 1H), 1.97 (td, J=5.1 Hz, J=12.9 Hz, 1H), 1.58 (dd, J=11.7 Hz, J=12.9 Hz, 1H); ¹³C NMR (CDCl₃; 63 MHz) δ 164.6 (Cq), 140.1 (Cq), 139.1 (Cq), 134.3 (Cq), 129.8 (CH), 128.3 (CH), 126.5 (Cq), 122.7 (CH), 65.2 (Cq), 57.0 (CH), 46.0 (CH₂), 40.2 (CH₂), 39.2 (CH₂), 38.9 (CH₂), 38.4 (CH₂).

### 2-Hydroxy-10-nitro-1,2,3,4-tetrahydropyrido[2,1-a]isoindol-6(10bH)-one (13)

NaBH₄ (155 mg, 4.08 mmol) was slowly added portionwise at −20° C. into a flask under an inert atmosphere, to a solution of the compound 8 (500 mg, 2.04 mmol) in a tetrahydrofuran/methanol mixture (10 mL/20 mL). The solution was stirred at this temperature for 1 hour and then at room temperature for 4 hours. Water (10 mL) was added and the aqueous phase was extracted first with dichloromethane (2×10 mL) and then with ethyl acetate (10 mL). The combined organic phases were dried, filtered and concentrated under reduced pressure. The raw residue was purified by flash chromatography on silica gel (CH₂Cl₂/MeOH 95:5) in order to obtain the compound 13 with a yield of 86%, as a white solid; m.p.>260° C.; ¹H NMR (DMSO-d₆, 400 MHz) δₚₚₘ: 8.39 (d, J=8.0 Hz, 1H), 8.11 (d, J=8.0 Hz, 1H), 7.79 (dd, J=8.0 Hz, 1H), 5.01-5.07 (m, 2H), 4.24 (dd, J=5.0 Hz, J=12.5 Hz, 1H), 3.89-3.98 (m, 1H), 3.08 (td, J=5.0 Hz, J=12.5 Hz, 1H), 2.64 (d, J=13.2 Hz, 1H), 1.93 (d, J=12.5 Hz, 1H), 1.04-1.21 (m, 1H), 0.76 (q, J=12.5 Hz, 1H). ¹³C NMR (DMSO-d₆, 100 MHz) δₚₚₘ 162.5 (Cq), 143.2 (Cq), 139.6 (Cq), 135.0 (Cq), 130.2 (CH), 129.5 (CH), 126.9 (CH), 66.9 (CH), 57.8 (CH), 38.2 (CH₂), 36.6 (CH₂), 33.9 (CH₂). IR (ATR-Ge v cm⁻¹): 2 973 (O—H), 1683 (C═O ketone), 1566 (C═C arom), 1329 (NO₂), 1288 (C—N). MS (IS) m/z: 249.0 [M+H]⁺.

### 2-Methoxy-10-nitro-1,2,3,4-tetrahydropyrido[2,1-a]isoindol-6(10bH)-one (14)

To the solution of the compound 13 (100 mg, 0.4 mmol) in anhydrous THF (5 mL) was added MeI (0.25 mL, 4.0 mmol) and freshly prepared Ag₂O (37 mg, 1.6 mmol). The mixture was stirred at 50° C. for 48 hours. After cooling down to room temperature, an Ag₂O precipitate was recovered by filtration and by suction, washed with dichloromethane (3×10 mL), and the combined organic filtrates were concentrated in vacuo. The residue was purified by silica gel chromatography (CH₂Cl₂/MeOH 99:1) in order to obtain the pure compound 14 as a white powder with a yield of 61%. m.p. 132-134° C.; ¹H NMR (CDCl₃, 400 MHz) δₚₚₘ: 8.33 (d, J=8.2 Hz, 1H), 8.15 (d, J=7.5 Hz, 1H), 7.67 (dd, J=7.5 Hz, J=8.0 Hz, 1H), 4.96 (dd, J=3.2 Hz, J=11.6 Hz, 1H), 4.56 (dd, J=5.0 Hz, J=13.2 Hz, 1H), 3.64 (m, 1H), 3.42 (s, 3H), 2.95-3.08 (m, 2H), 2.17 (d, J=13.2 Hz, 1H), 1.25-1.39 (m, 1H), 0.82 (q, J=12.5 Hz, 1H). ¹³C NMR (CDCl₃, 100 MHz) δₚₚₘ: 136.5 (Cq), 143.6 (Cq), 139.6 (Cq), 135.7 (Cq), 130.1 (CH), 129.9 (CH), 127.0 (CH), 76.8 (CH), 58.6 (CH), 56.1 (CH₃), 37.3 (CH₂), 35.0 (CH₂), 30.9 (CH₂). IR (ATR-Ge v cm⁻¹): 1688 (C═O ketone), 1523 (C═C arom), 1349 (NO₂), 1285 (C—N). MS (IS) m/z: 285.0 [M+Na]⁺.

### 10-Amino-2-methoxy-1,2,3,4-tetrahydropyrido[2,1-a]isoindol-6(10bH)-one (15)

Freshly prepared Raney W nickel (about 200 mg) was added to a stirred solution of the compound 14 (1.0 g, 3.8 mmol) in absolute ethanol (30 mL). The resulting suspension was hydrogenated at room temperature for 16 hours. The mixture was filtered on Celite and washed with dichloromethane (3×10 mL). The organic phase was concentrated in vacuo in order to obtain 815 mg (92%) of the compound 15 as a yellow solid, m.p. 60-62° C.; ¹H NMR (CDCl₃, 400 MHz) δₚₚₘ: 7.28 (m, 2H), 6.82 (d, J=8.5 Hz, 1H), 4.56 (dd, J=5.0 Hz, J=11.7 Hz, 1H), 4.33 (dd, J=5.0 Hz, J=12.0 Hz, 1H), 3.84 (bl, 2H), 3.52-3.64 (m, 1H), 3.43 (m, 3H), 2.96 (td, J=2.5 Hz, J=12.0 Hz, 1H), 2.81 (d, J=12.2 Hz, 1H), 2.18 (d, J=12.2 Hz, 1H), 1.34-1.44 (m, 1H), 1.05 (q, J=12.2 Hz, 1H). ¹³C NMR (CDCl₃, 100 MHz) δₚₚₘ: 166.5 (Cq), 141.1 (Cq), 133.1 (Cq), 129.3 (CH), 128.9 (Cq), 118.2 (CH), 114.1 (CH), 77.1 (CH), 56.1 (CH), 55.9 (CH₃), 36.9 (CH₂), 35.4 (CH₂), 30.8 (CH₂). IR (ATR-Ge v cm⁻¹): 3236 (NH₂), 1683 (C═O ketone), 1566 (C═C arom), 1288 (C—N). MS (IS) m/z: 233.0 [M+H]⁺.

2. Preparation of the Compounds of the Invention

2.1. General Procedure A

Synthesis of the ureas (16)-(61) from isocyanates and amines (5), (7), (10) or (15).

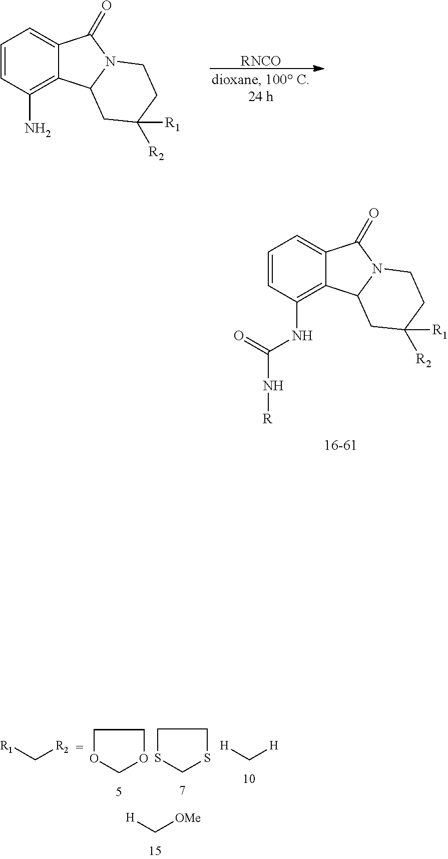

The compounds (16) to (61) hereafter are compounds of formula (I), in which R₄ and R″ are H and A is a CO group, and R₁ and R₂ are H, or H and OMe, or form together with the carbon atom to which they are attached, a group

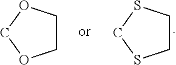

Synthesis of the isocyanates: Under an inert atmosphere, the carboxylic acid (0.37 mmol, 1.1 equ.) is dissolved in 7 mL of anhydrous THF and then 66 μL (0.48 mmol) of triethylamine are added. After 5 minutes of stirring at −10° C., 53 μL of ethyl chloroformate (0.55 mmol) are added. The mixture is stirred at this temperature for 1 hour. TLC analysis indicates complete conversion of the reaction. At this stage, an aqueous solution of NaN₃ (40 mg, 0.63 mmol) is added. The stirring is maintained for 1 hour at −10° C. The reaction medium is filtered, the filtrate is concentrated under reduced pressure and the aqueous phase is extracted with ethyl acetate (2×20 mL). The collected organic phases are dried on MgSO₄, filtered and then concentrated under reduced pressure in order to obtain a corresponding acyl nitrate, which is transformed by Curtius rearrangement in toluene (20 mL) with reflux into a corresponding isocyanate.

Synthesis of derivatives of ureas: To a solution of the freshly prepared isocyanate in 7 mL of anhydrous dioxane, the amine (0.34 mmol, 5, 7, 10 or 15) is added in one portion and the reaction mixture is then heated to 100° C. for 24 hours. After cooling, the solvent is evaporated under reduced pressure and the raw product is purified by silica gel chromatography in order to obtain the expected ureas.

### 1-(6′-oxo-3′,4′,6′,10b′-tetrahydro-1′H-spiro[[1,3]dithiolane-2,2′-pyrido[2,1-a]isoindole]-10′-yl)-3-(pyridin-2-yl)urea (16)

The compound 16 is obtained as a white solid according to the general procedure A with a yield of 96% after silica gel purification (CH₂Cl₂/MeOH: 99/1). MP: 242° C.; IR (ATR-Ge v cm⁻¹): 1703-1677 (C═O amide), 1553 (N—H amide), 1510-1483-1418 (C═C arom), 1310 (C—N), 1153 (C—C); ¹H NMR (DMSO-d6; 400 MHz) (δ 1.34 (s, 1H), 9.98 (s, 1H), 8.47 (d, J=3.9 Hz, 1H), 8.21 (d, J=7.9 Hz, 1H), 7.84-7.74 (m, 1H), 7.48 (t, J=7.7 Hz, 1H), 7.42 (d, J=6.9 Hz, 1H), 7.23 (d, J=8.3 Hz, 1H), 7.10-7.04 (m, 1H), 4.80 (dd, J=3.3 Hz, J=11.5 Hz, 1H), 4.34 (dd, J=3.2 Hz, J=13.7 Hz, 1H), 3.49-3.23 (m, 4H), 3.17 (td, J=3.2 Hz, J=13.3 Hz, 1H), 2.97 (d, J=11.8 Hz, 1H), 2.14 (d, J=13.3 Hz, 1H), 1.96 (td, J=5.0 Hz, J=13.0 Hz, 1H), 1.67 (t, J=12.2 Hz, 1H); ¹³C NMR (DMSO-d6; 101 MHz) (δ 164.8 (Cq), 152.9 (Cq), 152.2 (Cq), 146.4 (CH), 139.0 (CH), 134.1 (Cq), 133.8 (Cq), 132.6 (Cq), 129.0 (CH), 123.6 (CH), 117.7 (CH), 117.3 (CH), 112.1 (CH), 65.5 (Cq), 56.8 (CH), 44.0 (CH₂), 40.6 (CH₂), 38.8 (CH₂), 38.2 (CH₂), 37.7 (CH₂); MS (IS) m/z: 413.5 [M+H]⁺

### 1-(6′-Oxo-3′,4′,6′,10b′-tetrahydro-1′H-spiro[[1,3]dithiolane-2,2′-pyrido[2,1-a]isoindole]-10′-yl)-3-(pyrazin-2-yl)urea (17)

The compound 17 is obtained as a white solid according to the general procedure A with a yield of 90% after silica gel purification (CH₂Cl₂/MeOH: 99/1). MP: 236° C.; IR (ATR-Ge v cm⁻¹): 1676 (C═O amide), 1551 (N—H amide), 1505-1483-1421 (C═C arom), 1298 (C—N), 1138 (C—C); ¹H NMR (DMSO-d6; 400 MHz) (δ 10.19 (s, 1H), 10.13 (s, 1H), 8.83 (d, J=1.1 Hz, 1H), 8.40 (dd, J=1.5 Hz, J=2.6 Hz, 1H), 8.29 (d, J=2.7 Hz, 1H), 8.08 (dd, J=0.9 Hz, J=7.8 Hz, 1H), 7.54-7.42 (m, 2H), 4.79 (dd, J=3.4 Hz, J=11.6 Hz, 1H), 4.33 (dd, J=3.1 Hz, J=13.7 Hz, 1H), 3.48-3.28 (m, 4H), 3.16 (td, J=3.1 Hz, J=13.1 Hz, 1H), 2.89 (d, J=11.5 Hz, 1H), 2.14 (d, J=13.2 Hz, 1H), 1.96 (td, J=5.0 Hz, J=12.9 Hz, 1H), 1.63 (t, J=12.3 Hz, 1H); ¹³C NMR (DMSO-d6; 101 MHz) (δ 164.68 (Cq), 151.7 (Cq), 149.2 (Cq), 140.8 (CH), 137.6 (CH), 135.6 (CH), 134.7 (Cq), 133.3 (Cq), 132.7 (Cq), 129.1 (CH), 124.0 (CH), 118.3 (CH), 65.6 (Cq), 56.9 (CH), 44.0 (CH₂), 40.5 (CH₂), 38.7 (CH₂), 38.2 (CH₂), 37.7 (CH₂); MS (IS) m/z: 414.0 [M+H]⁺.

### 1-(5-Bromopyridin-2-yl)-3-(6-oxo-1,2,3,4,6,10b-hexahydropyrido[2,1-a]isoindol-10-yl)urea (18)

The compound 18 is obtained as a beige solid according to the general procedure A with a yield of 66% after silica gel purification (CH₂Cl₂/MeOH: 99/1). MP: 225° C.; IR (ATR-Ge v cm⁻¹): 1684 (C═O amide), 1560 (N—H amide), 1498-1465-1445 (C═C arom), 1362 (C—N), 1298-1232 (C—C), 749 (C—H arom); ¹H NMR (DMSO-d6; 400 MHz) δ 10.25 (s, 1H), 10.00 (s, 1H), 8.40 (d, J=2.3 Hz, 1H), 8.14 (dd, J=0.7 Hz, J=7.9 Hz, 1H), 8.00 (dd, J=2.5 Hz, J=8.9 Hz, 1H), 7.50 (d, J=8.9 Hz, 1H), 7.45 (t, J=7.7 Hz, 1H), 7.39 (dd, J=0.8 Hz, J=7.4 Hz, 1H), 4.59 (dd, J=3.2 Hz, J=11.5 Hz, 1H), 4.25 (dd, J=4.2 Hz, J=13.0 Hz, 1H), 3.00-3.11 (m, 1H), 2.63 (dd, J=2.7 Hz, J=12.6 Hz, 1H), 1.90 (d, J=12.6 Hz, 1H), 1.82-1.62 (m, 2H), 1.33-1.20 (m, 1H), 0.93-0.77 (m, 1H); ¹³C NMR (DMSO-d6; 101 MHz) δ 164.5 (Cq), 151.8 (Cq), 151.7 (Cq), 147.1 (CH), 141.2 (CH), 135.2 (Cq), 133.7 (Cq), 132.9 (Cq), 128.8 (CH), 122.5 (CH), 117.6 (CH), 113.9 (CH), 111.7 (Cq), 57.2 (CH), 45.6 (CH₂), 30.0 (CH₂), 25.0 (CH₂), 22.9 (CH₂); MS (IS) m/z: 402 [M+H]⁺

### 1-(6-Bromopyridin-2-yl)-3-(6-oxo-1,2,3,4,6,10b-hexahydropyrido[2,1-a]isoindol-10-yl)urea (19)

The compound 19 is obtained as a beige solid according to the general procedure A with a yield of 70% after silica gel purification (CH₂Cl₂/MeOH: 99/1). MP: 218-220° C.; IR (ATR-Ge v cm⁻¹): 1678 (C═O amide), 1538 (N—H amide), 1458-1429-1390 (C═C arom), 1337 (C—N), 1281-1251 (C—C), 748 (C—Br); ¹H NMR (DMSO-d6; 400 MHz) δ 10.04 (s, 1H), 9.28 (s, 1H), 8.10 (dd, J=0.7 Hz, J=7.8 Hz, 1H), 7.75-7.65 (m, 2H), 7.46 (t, J=7.7 Hz, 1H), 7.43-7.38 (m, 1H), 7.27 (dd, J=1.4 Hz, J=6.8 Hz, 1H), 4.56 (dd, J=3.4 Hz, J=11.5 Hz, 1H), 4.26 (dd, J=3.6 Hz, J=13.0 Hz, 1H), 3.02 (td, J=3.6 Hz, J=12.9 Hz, 1H), 2.66-2.55 (m, 1H), 1.89 (d, J=13.0 Hz, 1H), 1.81-1.61 (m, 2H), 1.34-1.20 (m, 1H), 0.87 (qd, J=3.1 Hz, J=12.7 Hz, 1H); ¹³C NMR (DMSO-d6; 101 MHz) δ 164.5 (Cq), 152.9 (Cq), 151.4 (Cq), 141.5 (CH), 138.1 (Cq), 135.2 (Cq), 133.5 (Cq), 132.9 (Cq), 128.8 (CH), 122.9 (CH), 121.2 (CH), 117.7 (CH), 110.7 (CH), 57.2 (CH), 39.0 (CH₂), 29.8 (CH₂), 25.0 (CH₂), 23.0 (CH₂); MS (IS) m/z: 402 [M+H]⁺.

### 1-(6-Oxo-1,2,3,4,6,10b-hexahydropyrido[2,1-a]isoindol-10-yl)-3-(quinolin-2-yl)urea (20)

The compound 20 is obtained as a white solid according to general procedure A with a yield of 67% after silica gel purification (CH₂Cl₂/MeOH: 99/1). MP: 260° C.; IR (ATR-Ge v cm⁻¹): 1691 (C═O amide), 1563 (N—H amide), 1432-1424 (C═C arom), 1256-1040 (C—C), 826-753 (C—H arom); ¹H NMR (CDCl₃; 400 MHz) (δ 12.32 (s, 1H), 10.24 (s, 1H), 8.35 (d, J=8.0 Hz, 1H), 7.94 (d, J=8.8 Hz, 1H), 7.71 (dd, J=6.2 Hz, J=15.8 Hz, 4H), 7.54 (t, J=7.7 Hz, 1H), 7.48-7.44 (m, 1H), 7.10 (d, J=8.8 Hz, 1H), 4.56 (dd, J=4.0 Hz, J=13.2 Hz, 1H), 4.42 (dd, J=3.0 Hz, J=11.7 Hz, 1H), 2.98 (td, J=3.0 Hz, J=12.9 Hz, 1H), 2.70 (d, J=10.5 Hz, 1H), 1.82 (t, J=15.1 Hz, 2H), 1.59 (q, J=13.2 Hz, 1H), 1.47-1.31 (m, 1H), 1.05 (qd, J=4.0 Hz, J=12.0 Hz, 1H); ¹³C NMR (CDCl₃; 101 MHz) (δ 166.0 (Cq), 154.3 (Cq), 152.3 (Cq), 145.0 (Cq), 139.1 (CH), 135.9 (Cq), 133.7 (Cq), 133.4 (Cq), 130.4 (CH), 129.2 (CH), 128.2 (CH), 126.2 (CH), 125.0 (CH), 125.0 (Cq), 124.5 (CH), 119.5 (CH), 114.0 (CH), 58.4 (CH), 40.0 (CH₂), 29.8 (CH₂), 25.4 (CH₂), 23.9 (CH₂); MS (IS) m/z: 373.0 [M+H]⁺

### 1-(6-Bromopyridin-2-yl)-3-(6′-oxo-3′,4′,6′,10b′-tetrahydro-1′H-spiro[[1,3]dithiolane-2,2′-pyrido[2,1-a]isoindole]-10′-yl)urea (21)

The compound 21 is obtained as a yellow solid according to the general procedure A with a yield of 57% after silica gel purification (CH₂Cl₂/MeOH: 99/1). MP: 170° C.; IR (ATR-Ge v cm⁻¹): 1654 (C═O amide), 1565 (N—H amide), 1530-1486-1430 (C═C arom), 786 (C—Br); ¹H NMR (CDCl₃; 400 MHz) (δ 10.89 (s, 1H), 9.93 (s, 1H), 7.74 (dd, J=7.6 Hz, J=13.8 Hz, 2H), 7.52 (td, J=5.7 Hz, J=7.8 Hz, 2H), 7.15 (d, J=7.7 Hz, 2H), 4.98 (dd, J=3.3 Hz, J=11.5 Hz, 1H), 4.56 (dd, J=3.7 Hz, J=13.8 Hz, 1H), 3.32-3.22 (m, 4H), 3.22-3.15 (m, 1H), 2.86 (d, J=12.0 Hz, 1H), 2.19 (d, J=13.5 Hz, 1H), 2.04 (td, J=5.0 Hz, J=13.4 Hz, 1H), 1.67 (t, J=12.0 Hz, 1H); ¹³C NMR (CDCl₃; 101 MHz) (δ 166.1 (Cq), 153.6 (Cq), 153.1 (Cq), 140.8 (CH), 138.2 (Cq), 137.3 (Cq), 133.7 (Cq), 132.4 (Cq), 129.4 (CH), 127.3 (CH), 121.4 (CH), 120.9 (CH), 111.2 (CH), 65.6 (Cq), 58.2 (CH), 45.2 (CH₂), 40.9 (CH₂), 39.0 (CH₂), 38.9 (CH₂), 38.6 (CH₂); MS (IS) m/z: decomposition.

### 1-(3-Methylpyridin-2-yl)-3-(6-oxo-1,2,3,4,6,10b-hexahydropyrido[2,1-a]isoindol-10-yl)urea (22)

The compound 22 is obtained as a white solid according to the general procedure A with a yield of 58% after purification on silica gel (CH₂Cl₂/MeOH: 99/1). MP: 235° C.; IR (ATR-Ge v cm⁻¹): 1681 (C═O amide), 1565 (N—H amide), 1482-1459-1434-1416 (C═C arom), 1286 (C—N), 1235 (C—C), 749 (C—H arom); ¹H NMR (CDCl₃; 400 MHz) (δ 12.29 (s, 1H), 8.26 (d, J=7.9 Hz, 1H), 8.09 (dd, J=1.1 Hz, J=5.0 Hz, 1H), 7.60 (dd, J=0.6 Hz, J=7.4 Hz, 1H), 7.53 (dd, J=0.7 Hz, J=7.3 Hz, 1H), 7.49-7.41 (m, 2H), 6.95 (dd, J=5.1 Hz, J=7.4 Hz, 1H), 4.54 (dd, J=4.9 Hz, J=13.3 Hz, 1H), 4.46 (dd, J=3.5 Hz, J=11.7 Hz, 1H), 3.02 (td, J=3.5 Hz, J=13.0 Hz, 1H), 2.81 (dd, J=3.1 Hz, J=13.0 Hz, 1H), 2.34 (s, 3H), 2.04 (d, J=13.4 Hz, 1H), 1.83 (d, J=13.2 Hz, 1H), 1.71 (qt, J=3.2 Hz, J=13.3 Hz, 1H), 1.54-1.36 (m, 1H), 1.08 (qd, J=3.1 Hz, J=13.0 Hz, 1H); ¹³C NMR (CDCl₃; 101 MHz) δ 166.1 (Cq), 152.8 (Cq), 151.3 (Cq), 143.0 (CH), 139.8 (CH), 135.5 (Cq), 133.5 (Cq), 133.4 (Cq), 129.2 (CH), 123.6 (CH), 120.0 (Cq), 119.0 (CH), 117.8 (CH), 58.5 (CH), 39.9 (CH₂), 30.3 (CH₂), 25.4 (CH₂), 24.1 (CH₂), 17.1 (CH₃); MS (IS) m/z: 337.0 [M+H]⁺.

### 1-(6-Fluoropyridin-2-yl)-3-(6-oxo-1,2,3,4,6,10b-hexahydropyrido[2,1-a]isoindol-10-yl)urea (23)

The compound 23 is obtained as a white solid according to the general procedure A with a yield of 70% after silica gel purification (CH₂Cl₂/MeOH: 99/1). MP: 254° C.; IR (ATR-Ge v cm⁻¹): 1687 (C═O amide), 1585 (N—H amide), 1561-1471-1420 (C═C arom), 1286 (C—F); ¹H NMR (DMSO-d6; 250 MHz) δ 9.92 (s, 1H), 9.33 (s, 1H), 8.14 (dd, J=1.2 Hz, J=7.6 Hz, 1H), 7.94 (q, J=8.2 Hz, 1H), 7.57 (dd, J=2.1 Hz, J=8.0 Hz, 1H), 7.51-7.36 (m, 2H), 6.77 (dd, J=2.0 Hz, J=7.9 Hz, 1H), 4.51 (dd, J=3.3 Hz, J=11.5 Hz, 1H), 4.26 (dd, J=3.5 Hz, J=13.0 Hz, 1H), 3.02 (td, J=3.5 Hz, 1J=2.7 Hz, 1H), 2.75-2.57 (m, 1H), 1.90 (d, J=13.2 Hz, 1H), 1.76 (d, J=14.1 Hz, 1H), 1.72-1.52 (m, 1H), 1.40-1.12 (m, 1H), 1.03-0.70 (m, 1H); ¹³C NMR (DMSO-d6; 63 MHz) δ 164.5 (Cq), 151.5 (Cq), 151.3 (Cq), 151.1 (Cq), 144.0 (CH), 135.1 (Cq), 133.6 (Cq), 132.9 (Cq), 128.8 (CH), 122.5 (CH), 117.6 (CH), 108.7 (CH), 101.3 (CH), 57.3 (CH), 39.1 (CH₂), 29.9 (CH₂), 24.9 (CH₂), 23.0 (CH₂); MS (IS) m/z: 341.0 [M+H]⁺, 363.0 [M+Na]⁺, 681.5 [2M+H]⁺.

### 1-(5-Méthylisoxazol-3-yl)-3-(6-oxo-1,2,3,4,6,10b-hexahydropyrido[2,1-a]isoindol-10-yl)urea (24)

The compound 24 is obtained as a yellow solid according to the general procedure A with a yield of 72% after silica gel purification (CH₂Cl₂/MeOH: 99/1). MP: 156° C.; IR (ATR-Ge v cm⁻¹): 1680 (C═O amide), 1624 (C═N), 1599 (N—H amide), 1539-1484-1428 (C═C arom), 1286 (C—O), 752 (C—H arom); ¹H NMR (CDCl₃; 400 MHz) δ 9.53 (s, 1H), 9.33 (s, 1H), 7.99 (d, J=7.9 Hz, 1H), 7.64 (d, J=7.3 Hz, 1H), 7.44 (t, J=7.8 Hz, 1H), 6.12 (s, 1H), 4.49 (td, J=4.0 Hz, J=11.8 Hz, 2H), 2.98 (td, J=3.2 Hz, J=13.0 Hz, 1H), 2.70 (d, J=11.1 Hz, 1H), 2.41 (s, 3H), 1.94 (d, J=13.3 Hz, 1H), 1.80 (d, J=13.1 Hz, 1H), 1.70-1.53 (m, 1H), 1.46-1.29 (m, 1H), 1.03 (qd, J=3.1 Hz, J=12.8 Hz, 1H); ¹³C NMR (CDCl₃; 101 MHz) δ 169.87 (Cq), 166.22 (Cq), 159.04 (Cq), 152.78 (Cq), 136.75 (Cq), 133.55 (Cq), 133.00 (Cq), 129.28 (CH), 124.80 (CH), 119.95 (CH), 95.36 (CH), 58.73 (CH), 40.13 (CH₂), 30.31 (CH₂), 25.43 (CH₂), 23.68 (CH₂), 12.62 (CH₃); MS (IS) m/z: 327.0 [M+H]⁺, 349.0 [M+Na]⁺, 653.5 [2M+H]⁺.

### 1-(6-oxo-1,2,3,4,6,10b-hexahydropyrido[2,1-a]isoindol-10-yl)-3-(5-phenyl-isoxazol-3-yl)urea (25)

The compound 25 is obtained as a white solid according to the general procedure A with a yield of 87% after silica gel purification (CH₂Cl₂/MeOH: 99/1). MP: 248° C.; IR (ATR-Ge v cm⁻¹): 1708 (C═O amide), 1680 (C═N), 1627 (N—H amide), 1535-1521-1485 (C═C arom), 1366 (C—N), 1288 (C—O), 746 (C—H arom); ¹H NMR (DMSO-d6; 400 MHz) δ 10.04 (s, 1H), 8.58 (s, 1H), 8.04 (d, J=7.7 Hz, 1H), 7.88 (dd, J=1.7 Hz, J=7.6 Hz, 2H), 7.59-7.39 (m, 5H), 7.27 (s, 1H), 4.52 (dd, J=3.3 Hz, J=11.5 Hz, 1H), 4.26 (dd, J=3.6 Hz, J=13.0 Hz, 1H), 3.02 (td, J=3.6 Hz, J=12.8 Hz, 1H), 2.61 (dd, J=2.9 Hz, J=12.5 Hz, 1H), 1.91 (d, J=13.1 Hz, 1H), 1.76 (d, J=12.5 Hz, 1H), 1.71-1.54 (m, 1H), 1.34-1.17 (m, 1H), 0.86 (qd, J=2.9 Hz, J=12.7 Hz, 1H); ¹³C NMR (DMSO-d6; 101 MHz) δ 168.5 (Cq), 164.4 (Cq), 159.1 (Cq), 151.2 (Cq), 135.5 (Cq), 133.6 (Cq), 133.0 (Cq), 130.5 (CH), 129.2 (2×CH), 128.7 (CH), 126.8 (Cq), 125.4 (2×CH), 123.0 (CH), 117.8 (CH), 93.7 (CH), 57.3 (CH), 39.0 (CH₂), 29.8 (CH₂), 25.0 (CH₂), 23.0 (CH₂); MS (IS) m/z: 389.0 [M+H]⁺, 411.0 [M+Na]⁺, 777.0 [2M+H]⁺.

### 1-(6-Oxo-1,2,3,4,6,10b-hexahydropyrido[2,1-a]isoindol-10-yl)-3-(thiazol-4-yl)urea (26)

The compound 26 is obtained as a yellow solid according to the general procedure A with a yield of 62% after silica gel purification (CH₂Cl₂/MeOH: 99/1). MP: 178-180° C.; IR (ATR-Ge v cm⁻¹): 3305-3224 (N—H amide), 1651 (C═O amide), 1529-1485-1427 (C═C arom), 1288 (C—N), 1200 (C—C), 723 (C—H arom); ¹H NMR (CDCl₃; 400 MHz) δ 9.68 (s, 1H), 8.64 (d, J=1.5 Hz, 1H), 7.96 (d, J=7.8 Hz, 1H), 7.61 (d, J=7.4 Hz, 1H), 7.41 (t, J=7.8 Hz, 1H), 7.11 (s, 1H), 4.57-4.38 (m, 2H), 2.97-2.91 (m, 1H), 2.65 (d, J=10.9 Hz, 1H), 1.88 (d, J=12.2 Hz, 1H), 1.76 (d, J=12.3 Hz, 1H), 1.60-1.43 (m, 1H), 1.43-1.28 (m, 1H), 1.10-0.92 (m, 1H); ¹³C NMR (CDCl₃; 101 MHz) δ 166.4 (Cq), 153.0 (Cq), 150.8 (CH), 149.9 (Cq), 136.6 (Cq), 133.5 (Cq), 133.3 (Cq), 129.2 (CH), 124.9 (CH), 119.4 (CH), 97.7 (CH), 58.91 (CH), 40.1 (CH₂), 30.3 (CH₂), 25.4 (CH₂), 23.6 (CH₂); MS (IS) m/z: 329.0 [M+H]⁺, 351.0 [M+Na]⁺, 657.5 [2M+H]⁺.

### 1-(4-Methylpyridin-2-yl)-3-(6-oxo-1,2,3,4,6,10b-hexahydropyrido[2,1-a]isoindol-10-yl)urea (27)

The compound 27 is obtained as a white solid according to the general procedure A with a yield of 90% after silica gel purification (CH₂Cl₂/MeOH: 99/1). MP: 178-180° C.; IR (ATR-Ge v cm⁻¹): 3305-3224 (N—H amide), 1651 (C═O amide), 1529-1485-1427 (C═C arom), 1288 (C—N), 1200 (C—C), 723 (C—H arom); ¹H NMR (CDCl₃; 400 MHz) δ 9.68 (s, 1H), 8.64 (d, J=1.5 Hz, 1H), 7.96 (d, J=7.8 Hz, 1H), 7.61 (d, J=7.4 Hz, 1H), 7.41 (t, J=7.8 Hz, 1H), 7.11 (s, 1H), 4.57-4.38 (m, 2H), 2.97-2.91 (m, 1H), 2.65 (d, J=10.9 Hz, 1H), 1.88 (d, J=12.2 Hz, 1H), 1.76 (d, J=12.3 Hz, 1H), 1.60-1.43 (m, 1H), 1.43-1.28 (m, 1H), 1.10-0.92 (m, 1H); ¹³C NMR (CDCl₃; 101 MHz) δ: 166.4 (Cq), 153.0 (Cq), 150.8 (CH), 149.9 (Cq), 136.6 (Cq), 133.5 (Cq), 133.3 (Cq), 129.2 (CH), 124.9 (CH), 119.4 (CH), 97.7 (CH), 58.9 (CH), 40.1 (CH₂), 30.3 (CH₂), 25.4 (CH₂), 23.6 (CH₂); MS (IS) m/z: 329.0 [M+H]⁺, 351.0 [M+Na]⁺, 657.5 [2M+H]⁺.

### 1-(6′-Oxo-3′,4′,6′,10b′-tetrahydro-1′H-spiro[[1,3]dioxolane-2,2′-pyrido[2,1-a]isoindole]-10′-yl)-3-(pyridin-2-yl)urea (28)

The compound 28 is obtained as a white solid according to the general procedure A with a yield of 78% after silica gel purification (CH₂Cl₂/MeOH: 99/1). MP: 178-180° C.; IR (ATR-Ge v cm⁻¹): 3305-3224 (N—H amide), 1651 (C═O amide), 1529-1485-1427 (C═C arom), 1288 (C—N), 1200 (C—C), 723 (C—H arom); ¹H NMR (CDCl₃; 400 MHz) δ 9.68 (s, 1H), 8.64 (d, J=1.5 Hz, 1H), 7.96 (d, J=7.8 Hz, 1H), 7.61 (d, J=7.4 Hz, 1H), 7.41 (t, J=7.8 Hz, 1H), 7.11 (s, 1H), 4.57-4.38 (m, 2H), 2.97-2.91 (m, 1H), 2.65 (d, J=10.9 Hz, 1H), 1.88 (d, J=12.2 Hz, 1H), 1.76 (d, J=12.3 Hz, 1H), 1.60-1.43 (m, 1H), 1.43-1.28 (m, 1H), 1.10-0.92 (m, 1H); ¹³C NMR (CDCl₃; 101 MHz) δ 166.4 (Cq), 153.0 (Cq), 150.8 (CH), 149.9 (Cq), 136.6 (Cq), 133.5 (Cq), 133.3 (Cq), 129.2 (CH), 124.9 (CH), 119.4 (CH), 97.7 (CH), 58.9 (CH), 40.1 (CH₂), 30.3 (CH₂), 25.4 (CH₂), 23.6 (CH₂); MS (IS) m/z: 329.0 [M+H]⁺, 351.0 [M+Na]⁺, 657.5 [2M+H]⁺.

### 1-(6′-Oxo-3′,4′,6′,10b′-tetrahydro-1′H-spiro[[1,3]dioxolane-2,2′-pyrido[2,1-a]isoindole]-10′-yl)-3-(pyrazin-2-yl)urea (29)

The compound 29 is obtained as a pale brown solid according to the general procedure A with a yield of 68% after silica gel purification (CH₂Cl₂/MeOH: 99/1). F>260° C.; IR (ATR-Ge v cm⁻¹): 1698 (C═O amide), 1570 (N—H amide), 1549-1504-1478 (C═C arom), 1298 (C—O), 1244 (C—N), 1073 (C—C); ¹H NMR (DMSO-d6; 400 MHz) δ 10.12 (s, 1H), 10.03 (s, 1H), 8.88 (s, 1H), 8.34-8.25 (m, 2H), 8.13 (d, J=7.8 Hz, 1H), 7.49 (t, J=7.7 Hz, 1H), 7.43 (d, J=6.9 Hz, 1H), 4.78 (dd, J=3.6 Hz, J=12.0 Hz, 1H), 4.28 (dd, J=4.0 Hz, J=13.3 Hz, 1H), 4.14-4.00 (m, 2H), 4.00-3.83 (m, 2H), 3.15 (td, J=4.0 Hz, J=13.1 Hz, 1H), 2.55 (d, J=11.5 Hz, 1H), 1.83 (d, J=13.1 Hz, 1H), 1.51 (td, J=5.7 Hz, 13.0 Hz, 1H), 1.23 (t, J=12.3 Hz, 1H); ¹³C NMR (DMSO-d6; 101 MHz) δ 164.7 (Cq), 151.6 (Cq), 149.2 (Cq), 140.7 (CH), 137.8 (CH), 135.5 (CH), 134.8 (Cq), 133.4 (Cq), 132.8 (Cq), 129.0 (CH), 123.2 (CH), 118.0 (CH), 106.8 (Cq), 64.2 (CH₂), 63.9 (CH₂), 55.5 (CH), 38.0 (CH₂), 36.0 (CH₂), 33.1 (CH₂); MS (IS) m/z: 382.0 [M+H]⁺, 404.0 [M+Na]⁺, 763.5 [2M+H]⁺.

### 1-(6-oxo-1,2,3,4,6,10b-hexahydropyrido[2,1-a]isoindol-10-yl)-3-(pyridin-2-yl)urea (30)

The compound 30 is obtained as a white solid according to the general procedure A with a yield of 90% after silica gel purification (CH₂Cl₂/MeOH 97/3+1% Et₃N). MP: 142° C.; IR (ATR-Ge v cm⁻¹): 3515 (NH amide), 1670 (C═O amide), 1603 (N—H amide), 1576-1478-1417 (C═C arom), 1315 (C—N); ¹H NMR (DMSO-d6; 400 MHz) δ 11.16 (s, 1H), 10.00 (s, 1H), 8.29 (d, J=4.0 Hz, 1H), 8.23 (d, J=8.0 Hz, 1H), 7.85-7.74 (m, 1H), 7.45 (t, J=7.7 Hz, 1H), 7.38 (dd, J=0.8 Hz, J=7.4 Hz, 1H), 7.33 (d, J=8.3 Hz, 1H), 7.11-7.03 (m, 1H), 4.59 (dd, J=3.3 Hz, J=11.5 Hz, 1H), 4.26 (dd, J=4.4 Hz, J=13.0 Hz, 1H), 3.12-2.97 (m, 1H), 2.72 (dd, J=3.0 Hz, J=12.6 Hz, 1H), 1.91 (d, J=13.3 Hz, 1H), 1.83-1.64 (m, 2H), 1.36-1.22 (m, 1H), 0.95-0.77 (qd, 1H, J=13.0 Hz, J=3.0 Hz); ¹³C NMR (DMSO-d6; 101 MHz) δ 164.6 (Cq), 152.7 (Cq), 152.1 (Cq), 145.9 (CH), 139.2 (CH), 134.8 (Cq), 133.9 (Cq), 132.8 (Cq), 128.8 (CH), 122.2 (CH), 117.6 (CH), 117.3 (CH), 112.2 (CH), 57.3 (CH), 39.0 (CH₂), 30.0 (CH₂), 25.0 (CH₂), 23.0 (CH₂); MS (IS) m/z: 323.5 [M+H]⁺.

### 1-(5-Bromopyridin-2-yl)-3-(6′-oxo-3′,4′,6′,10b′-tetrahydro-1′H-spiro[[1,3]dithiolane-2,2′-pyrido[2,1-a]isoindole]-10′-yl)urea (31)

The compound 31 is obtained as a yellow solid according to the general procedure A with a yield of 82% after silica gel purification (CH₂Cl₂/MeOH: 99/1). MP: 240° C.; IR (ATR-Ge v cm⁻¹): 1700 (C═O amide), 1551 (N—H amide), 1481-1430 (C═C arom), 1367 (C—N), 1240-1048 (C—C), 759 (C—Br); ¹H NMR (DMSO-d6; 400 MHz) δ 10.69 (s, 1H), 10.10 (s, 1H), 8.57 (d, J=2.4 Hz, 1H), 8.17 (d, J=7.8 Hz, 1H), 8.01 (dd, J=2.5 Hz, 8.9 Hz, 1H), 7.49 (t, J=7.7 Hz, 1H), 7.43 (d, J=6.8 Hz, 1H), 7.33 (d, J=8.9 Hz, 1H), 4.78 (dd, J=3.3 Hz, J=11.6 Hz, 1H), 4.34 (dd, J=3.2 Hz, J=13.5 Hz, 1H), 3.50-3.27 (m, 4H), 3.17 (td, J=3.2 Hz, J=13.3 Hz, 1H), 2.92 (d, J=11.7 Hz, 1H), 2.15 (d, J=13.3 Hz, 1H), 1.95 (td, J=5.0 Hz, J=12.9 Hz, 1H), 1.66 (t, J=12.2 Hz, 1H); ¹³C NMR (DMSO-d6; 101 MHz) δ 164.7 (Cq), 151.8 (2×Cq), 147.1 (CH), 141.3 (CH), 134.2 (Cq), 133.6 (Cq), 132.3 (Cq), 129.1 (CH), 123.6 (CH), 117.9 (CH), 114.0 (CH), 111.4 (Cq), 65.7 (Cq), 56.8 (CH), 43.9 (CH₂), 40.7 (CH₂), 38.7 (CH₂), 38.1 (CH₂), 37.7 (CH₂); MS (IS) m/z: 492.5 [M+H]⁺.

### 1-(6-Oxo-1,2,3,4,6,10b-hexahydropyrido[2,1-a]isoindol-10-yl)-3-(pyrimidin-4-yl)urea (32)

The compound 32 is obtained as a beige solid according to the general procedure A with a yield of 76% after silica gel purification (CH₂Cl₂/MeOH: 99/1). MP: 218° C.; IR (ATR-Ge v cm⁻¹): 1685 (C═O amide), 1559 (N—H amide), 1478-1435-1417-1389 (C═C arom), 1310 (C—N); ¹H NMR (DMSO-d6; 400 MHz) δ 10.21 (s, 1H), 10.14 (s, 1H), 8.84 (s, 1H), 8.58 (d, J=5.6 Hz, 1H), 8.12 (d, J=7.6 Hz, 1H), 7.54 (d, J=5.4 Hz, 1H), 7.5-7.4 (m, 2H), 4.57 (d, J=8.8 Hz, 1H), 4.26 (d, J=9.6 Hz, 1H), 3.03 (t, J=11.8 Hz, 1H), 2.64 (d, J=10.9 Hz, 1H), 1.91 (d, J=11.9 Hz, 1H), 1.80-1.61 (m, 2H), 1.38-1.12 (m, 1H), 0.91-0.82 (m, 1H); ¹³C NMR (DMSO-d6; 101 MHz) δ 164.4 (Cq), 158.0 (Cq), 157.6 (CH), 157.3 (CH), 151.4 (Cq), 135.4 (Cq), 133.2 (Cq), 133.0 (Cq), 128.9 (CH), 122.8 (CH), 118.0 (CH), 108.6 (CH), 57.3 (CH), 39.0 (CH₂), 29.9 (CH₂), 25.0 (CH₂), 22.9 (CH₂); MS (IS) m/z: 324.0 [M+H]⁺, 647.5 [2M+H]⁺.

### 1-(6-oxo-1,2,3,4,6,10b-hexahydropyrido[2,1-a]isoindol-10-yl)-3-(perchloro-pyridin-2-yl)urea (33)

The compound 33 is obtained as a beige solid according to the general procedure A with a yield of 60% after silica gel purification (CH₂Cl₂/MeOH: 99/1). MP: 246° C.; IR (ATR-Ge v cm⁻¹): 1683 (C═O amide), 1598 (N—H amide), 1529-1461-1430 (C═C arom), 1278 (C—N), 1221 (C—C), 750-732 (C—Cl); ¹H NMR (DMSO-d6; 400 MHz) δ 9.60 (d, J=2.6 Hz, 2H), 7.97 (dd, J=1.2 Hz, J=7.3 Hz, 1H), 7.49-7.44 (m, 2H), 4.58 (dd, J=3.3 Hz, J=11.5 Hz, 1H), 4.25 (dd, J=3.8 Hz, J=12.6 Hz, 1H), 3.02 (td, J=3.8 Hz, J=12.0 Hz, 1H), 2.61 (d, J=10.2 Hz, 1H), 1.88 (d, J=13.0 Hz, 1H), 1.75 (d, J=12.3 Hz, 1H), 1.64 (q, J=12.8 Hz, 1H), 1.29-1.15 (m, 1H), 0.92-0.84 (m, 1H); ¹³C NMR (DMSO-d6; 101 MHz) δ 164.4 (Cq), 150.4 (Cq), 147.3 (Cq), 143.9 (Cq), 143.0 (Cq), 136.2 (Cq), 133.1 (Cq), 133.0 (Cq), 128.7 (CH), 124.0 (CH), 122.3 (Cq), 119.2 (Cq), 118.4 (CH), 57.3 (CH), 39.1 (CH₂), 29.7 (CH₂), 25.0 (CH₂), 23.0 (CH₂); MS (IS) m/z: 461.0 [M+H]⁺, 483.0 [M+Na]⁺.

### 1-(Benzo[d]thiazol-2-yl)-3-(6-oxo-1,2,3,4,6,10b-hexahydropyrido[2,1-a]-isoindol-10-yl)urea (34)

The compound 34 is obtained as a pale yellow solid according to the general procedure A with a yield of 50% after silica gel purification (CH₂Cl₂/MeOH: 99/1). MP: 260° C.; IR (ATR-Ge v cm⁻¹): 1688 (C═O amide), 1597 (N—H amide), 1561-1524-1482 (C═C arom), 1286 (C—N); ¹H NMR (DMSO-d6; 400 MHz) δ 11.19 (s, 1H), 9.07 (s, 1H), 8.05 (d, J=7.0 Hz, 1H), 7.93 (d, J=7.7 Hz, 1H), 7.69 (d, J=6.6 Hz, 1H), 7.51-7.39 (m, 3H), 7.26 (t, J=7.5 Hz, 1H), 4.57 (d, J=9.4 Hz, 1H), 4.26 (d, J=9.2 Hz, 1H), 3.07-3.01 (m, 1H), 2.61 (d, J=11.5 Hz, 1H), 1.90 (d, J=12.8 Hz, 1H), 1.76 (d, J=12.4 Hz, 1H), 1.64 (q, J=13.0 Hz, 1H), 1.32-1.15 (m, 1H), 0.88 (q, J=12.0 Hz, 1H); ¹³C NMR (DMSO-d6; 101 MHz) δ 164.4 (Cq), 135.8 (2×Cq), 133.2 (2×Cq), 133.1 (Cq), 128.8 (CH), 126.0 (CH), 123.3 (CH+Cq), 123.0 (CH), 121.6 (CH), 120.0 (CH), 118.3 (CH), 57.3 (CH), 54.8 (Cq), 39.0 (CH₂), 29.8 (CH₂), 24.9 (CH₂), 23.0 (CH₂); MS (IS) m/z: 379.5 [M+H]⁺.

### 1-(2-Methylthiazol-4-yl)-3-(6-oxo-1,2,3,4,6,10b-hexahydropyrido[2,1-a]isoindol-10-yl)urea (35)

The compound 35 is obtained as a pale yellow solid according to the general procedure A with a yield of 42% after silica gel purification (CH₂Cl₂/MeOH: 99/1). MP: 146° C.; IR (ATR-Ge v cm⁻¹): 1691 (C═O amide), 1650 (N—H amide), 1532-1484-1429 (C═C arom), 1286 (C—N), 1202 (C—C); ¹H NMR (CDCl₃; 400 MHz) δ 9.30 (s, 2H), 8.03 (d, J=6.5 Hz, 1H), 7.62 (d, J=7.3 Hz, 1H), 7.41 (t, J=7.8 Hz, 1H), 6.70 (s, 1H), 4.51 (dd, J=4.0 Hz, J=13.2 Hz, 1H), 4.45 (dd, J=3.5 Hz, J=11.6 Hz, 1H), 2.95 (t, J=12.0 Hz, 1H), 2.71 (s, 4H), 1.92 (d, J=12.6 Hz, 1H), 1.78 (d, J=12.6 Hz, 1H), 1.62-1.48 (m, 1H), 1.47-1.31 (m, 1H), 1.04 (q, J=12.0 Hz, 1H); ¹³C NMR (CDCl₃; 101 MHz) δ 166.3 (Cq), 164.6 (Cq), 152.9 (Cq), 147.9 (Cq), 136.2 (Cq), 133.5 (Cq), 133.4 (Cq), 129.2 (CH), 124.7 (CH), 119.4 (CH), 96.6 (CH), 58.69 (CH), 40.1 (CH₂), 30.3 (CH₂), 25.4 (CH₂), 23.9 (CH₂), 19.3 (CH₃); MS (IS) m/z: 343.0 [M+H]⁺, 685.5 [2M+H]⁺.

### 1-(6-Methylpyridin-2-yl)-3-(6-oxo-1,2,3,4,6,10b-hexahydropyrido[2,1-a]isoindol-10-yl)urea (36)

The compound 36 is obtained as a white solid according to the general procedure A with a yield of 73% after silica gel purification (CH₂Cl₂/MeOH: 99/1). MP: 210° C.; IR (ATR-Ge v cm⁻¹): 1685 (C═O amide), 1572 (N—H amide), 1510-1464-1438 (C═C arom), 1283 (C—N), 757 (C—H arom); ¹H NMR (CDCl₃; 400 MHz) δ 1.95 (s, 1H), 9.56 (s, 1H), 8.13 (d, J=8.0 Hz, 1H), 7.67 (d, J=7.5 Hz, 1H), 7.56 (t, J=7.8 Hz, 1H), 7.49 (t, J=7.8 Hz, 1H), 6.84-6.76 (m, 2H), 4.65-4.46 (m, 2H), 3.00 (td, J=3.5 Hz, J=13.1 Hz, 1H), 2.71 (dd, J=3.2 Hz, 13.0 Hz, 1H), 1.96 (d, J=13.3 Hz, 1H), 1.81 (d, J=13.0 Hz, 1H), 1.63 (q, J=13.2 Hz, 1H), 1.51-1.34 (m, 1H), 1.10 (qd, J=3.2 Hz, J=13.0 Hz, 1H); ¹³C NMR (CDCl₃; 101 MHz) δ 166.1 (Cq), 155.0 (Cq), 154.2 (Cq), 152.6 (Cq), 139.2 (CH), 136.4 (Cq), 133.6 (Cq), 133.3 (Cq), 129.1 (CH), 125.2 (CH), 119.6 (CH), 117.0 (CH), 109.6 (CH), 58.6 (CH), 40.0 (CH₂), 29.9 (CH₂), 25.4 (CH₂), 24.5 (CH₃), 23.9 (CH₂); MS (IS) m/z: 337.5 [M+H]⁺.

### 1-(6-Oxo-1,2,3,4,6,10b-hexahydropyrido[2,1-a]isoindol-10-yl)-3-(2-(pyridin-4-yl)thiazol-4-yl)urea (37)

The compound 37 is obtained as a pale orange solid according to the general procedure A with a yield of 60% after silica gel purification (CH₂Cl₂/MeOH: 99/1). MP: 152° C.; IR (ATR-Ge v cm⁻¹): 1649 (C═O amide), 1561 (N—H amide), 1521-1485-1430 (C═C arom), 1201 (C—C); ¹H NMR (DMSO-d6; 400 MHz) δ 10.10 (s, 1H), 8.72 (dd, J=1.5 Hz, J=4.6 Hz, 2H), 8.48 (s, 1H), 8.11 (d, J=7.2 Hz, 1H), 7.84 (dd, J=1.6 Hz, J=4.5 Hz, 2H), 7.55 (s, 1H), 7.45 (t, J=7.7 Hz, 1H), 7.38 (dd, J=0.8 Hz, J=7.4 Hz, 1H), 4.50 (dd, J=3.3 Hz, J=11.5 Hz, 1H), 4.26 (dd, J=3.7 Hz, J=13.0 Hz, 1H), 3.03 (td, J=3.7 Hz, J=12.9 Hz, 1H), 2.62 (dd, J=2.9 Hz, J=12.6 Hz, 1H), 1.90 (d, J=13.1 Hz, 1H), 1.76 (d, J=12.6 Hz, 1H), 1.62 (q, J=13.0 Hz, 1H), 1.34-1.15 (m, 1H), 0.85 (qd, J=2.9 Hz, J=12.7 Hz, 1H); ¹³C NMR (DMSO-d6; 101 MHz) δ 164.5 (Cq), 161.4 (Cq), 151.6 (Cq), 150.8 (2×CH), 149.6 (Cq), 139.2 (Cq), 134.9 (Cq), 134.00 (Cq), 132.9 (Cq), 128.7 (CH), 122.3 (CH), 119.4 (2×CH), 117.3 (CH), 100.4 (CH), 57.3 (CH), 39.0 (CH₂), 29.9 (CH₂), 25.0 (CH₂), 23.0 (CH₂); MS (IS) m/z: 406.5 [M+H]⁺.

### 1-(6-Oxo-1,2,3,4,6,10b-hexahydropyrido[2,1-a]isoindol-10-yl)-3-(pyrazin-2-yl)urea (38)

The compound 38 is obtained as a beige solid according to the general procedure A with a yield of 70% after silica gel purification (CH₂Cl₂/MeOH 96/4). MP: 215° C.; IR (ATR-Ge v cm⁻¹): 1699-1683 (C═O amide), 1671 (C═N), 1651 (N—H amide), 1558-1540-1520-1507 (C═C arom); ¹H NMR (DMSO-d6; 400 MHz) δ 10.05 (s, 1H), 9.81 (s, 1H), 8.93 (d, J=1.3 Hz, 1H), 8.36-8.31 (m, 1H), 8.29 (d, J=2.7 Hz, 1H), 8.13 (dd, J=1.0 Hz, J=7.9 Hz, 1H), 7.47 (t, J=7.7 Hz, 1H), 7.42 (dd, J=1.0 Hz, J=7.4 Hz, 1H), 4.56 (dd, J=3.4 Hz, J=11.5 Hz, 1H), 4.26 (dd, J=3.8 Hz, J=13.0 Hz, 1H), 3.03 (td, J=3.8 Hz, J=12.8 Hz, 1H), 2.63 (dd, J=3.0 Hz, J=12.6 Hz, 1H), 1.91 (d, J=13.1 Hz, 1H), 1.76 (d, J=15.0 Hz, 1H), 1.72-1.64 (m, 1H), 1.33-1.16 (m, 1H), 0.88 (qd, J=3.0 Hz, J=12.7 Hz, 1H); ¹³C NMR (DMSO-d6; 101 MHz) δ 164.4 (Cq), 151.6 (Cq), 149.1 (Cq), 141.0 (CH), 137.8 (CH), 135.4 (CH), 135.3 (Cq), 133.5 (Cq), 132.9 (Cq), 128.8 (CH), 122.8 (CH), 117.8 (CH), 57.3 (CH), 39.0 (CH₂), 29.9 (CH₂), 25.01 (CH₂), 23.0 (CH₂); MS (IS) m/z: 324.0 [M+H]⁺

### 1-(5-methylpyrazin-2-yl)-3-(6-oxo-1,2,3,4,6,10b-hexahydropyrido[2,1-a]isoindol-10-yl)urea (39)

The compound 39 is obtained according to the procedure A from the amine 10 as a white solid with 80% of yield after flash silica gel chromatography (CH₂Cl₂/MeOH: 99/1). MP: 220° C.; IR (ATR-Ge v cm⁻¹): 1687 (C═O amide), 1560 (N—H amide), 1503-1483-1416 (C═C arom), 1342 (C—N), 1155 (C—C); ¹H NMR (DMSO-d6; 400 MHz) δ 9.92 (s, 1H), 9.74 (s, 1H), 8.82 (s, 1H), 8.20 (s, 1H), 8.13 (d, J=7.8 Hz, 1H), 7.46 (t, J=7.7 Hz, 1H), 7.40 (d, J=7.2 Hz, 1H), 4.54 (dd, J=3.1 Hz, 11.5 Hz, 1H), 4.26 (dd, J=4.1 Hz, 13.0 Hz, 1H), 3.09-2.95 (m, 1H), 2.63 (d, J=10.1 Hz, 1H), 2.44 (s, 3H), 1.91 (d, J=13.2 Hz, 1H), 1.82-1.60 (m, 2H), 1.33-1.15 (m, 1H), 0.94-0.77 (m, 1H); ¹³C NMR (DMSO-d6; 101 MHz) δ 164.5 (Cq), 151.7 (Cq), 146.9 (Cq), 146.2 (Cq), 139.9 (CH), 135.1 (Cq), 134.0 (CH), 133.6 (Cq), 132.9 (Cq), 128.8 (CH), 122.6 (CH), 117.6 (CH), 57.3 (CH), 39.0 (CH₂), 29.9 (CH₂), 25.0 (CH₂), 23.0 (CH₂), 20.1 (CH₃). MS (IS) m/z: 338.5 [M+H]⁺, 675.5 [2M+H]⁺

### 1-(6′-Oxo-3′,4′,6′,10b′-tetrahydro-1′H-spiro[[1,3]dithiolane-2,2′-pyrido[2,1-a]isoindole]-10′-yl)-3-(1-Methyl-1H-pyrazol-3-yl)urea (40)

The compound 40 was obtained according to the procedure A after purification by flash chromatography (CH₂Cl₂/MeOH: 99/1) as a yellow solid. Yield: 68%. m.p. 135° C.; IR (ATR-Ge v cm⁻¹): 3256 (N—H amide), 2923 (Cₛₚ₃—H), 1676 (C═O amide), 1530-1485 (C═C arom), 1287 (C—N); ¹H NMR (CDCl₃; 400 MHz) δ 10.08 (s, 1H), 8.82 (s, 1H), 7.89 (d, J=8.0 Hz, 1H), 7.61 (d, J=7.4 Hz, 1H), 7.42 (t, J=7.7 Hz, 1H), 7.23 (d, J=2.2 Hz, 1H), 5.91 (s, 1H), 4.81 (dd, J=11.4 Hz, 3.1 Hz, 1H), 4.52 (dd, J=13.8 Hz, 3.8 Hz, 1H), 3.87 (s, 3H), 3.33-3.16 (m, 5H), 2.95 (d, J=12.5 Hz, 1H), 2.17 (d, J=12.1 Hz, 1H), 2.01 (td, J=13.0 Hz, 5.0 Hz, 1H), 1.62 (t, J=12.0 Hz, 1H); ¹³C NMR (CDCl₃; 101 MHz) δ 166.3 (Cq), 153.5 (Cq), 148.5 (Cq), 136.0 (Cq), 133.3 (Cq), 133.3 (Cq), 131.4 (CH), 129.3 (CH), 126.3 (CH), 119.8 (CH), 94.65 (CH), 65.8 (Cq), 57.8 (CH), 45.0 (CH₂), 41.0 (CH₂), 39.2 (CH₃), 38.9 (CH₂), 38.9 (CH₂), 38.4 (CH₂); HRMS: calculated for C₁₉H₂₁N₅O₂NaS₂ 438.1034; found 438.1025

### 1-(1-Methyl-1H-pyrazol-3-yl)-3-(6-oxo-1,2,3,4,6,10b-hexahydro-pyrido[2,1-a]isoindol-10-yl)-urea (41)

The compound 41 was obtained according to the procedure A after purification by flash chromatography (CH₂Cl₂/MeOH: 99/1) as a white solid. Yield: 62% m.p. 233° C.; IR (ATR-Ge v cm⁻¹): 3083 (Cₛₚ₂—H arom), 2937 (Cₛₚ₃—H), 1686 (C═O amide), 1598-1481 (C═C arom), 1284 (C—N); ¹H NMR (DMSO-d6; 400 MHz) δ 9.42 (s, 1H), 9.21 (s, 1H), 8.18 (d, J=7.3 Hz, 1H), 7.59 (s, 1H), 7.43 (t, J=7.7 Hz, 1H), 7.35 (d, J=7.2 Hz, 1H), 6.07 (s, 1H), 4.52 (d, J=8.9 Hz, 1H), 4.26 (d, J=9.5 Hz, 1H), 3.78 (s, 3H), 3.03 (t, J=11.6 Hz, 1H), 2.71 (d, J=11.1 Hz, 1H), 1.92 (d, J=13.1 Hz, 1H), 1.76 (d, J=12.5 Hz, 1H), 1.74-1.55 (m, 1H), 1.25 (d, J=12.6 Hz, 1H), 0.87 (dd, J=23.0 Hz, 11.8 Hz, 1H); ¹³C NMR (DMSO-d6; 101 MHz) δ 164.6 (Cq), 151.7 (Cq), 147.7 (Cq), 134.5 (Cq), 134.4 (Cq), 132.8 (Cq), 131.7 (CH), 128.7 (CH), 122.1 (CH), 117.0 (CH), 94.3 (CH), 57.3 (CH), 39.0 (CH₂), 38.3 (CH₃), 29.8 (CH₂), 25.0 (CH₂), 23.1 (CH₂); HRMS: calc. for C₁₇H₁₉N₅O₂Na 348.1436; found 348.1431.

### 1-(6-oxo-1,2,3,4,6,10b-hexahydro-pyrido[2,1-a]isoindol-10-yl)-3-pyrimidin-2-yl-urea (42)

The compound 42 was obtained according to the procedure A after is purification by flash chromatography (CH₂Cl₂/MeOH: 99/1) as a beige solid. Yield: 87%. m.p. 232° C.; IR (ATR-Ge v cm⁻¹): 3063 (Cₛₚ₂—H arom), 2913 (Cₛₚ₃—H), 1691 (C═O amide), 1582-1484 (C═C arom), 1291 (C—N); ¹H NMR (DMSO-d6; 400 MHz) δ 11.60 (s, 1H), 10.42 (s, 1H), 8.70 (d, J=4.9 Hz, 2H), 8.24 (d, J=7.8 Hz, 1H), 7.48 (t, J=7.7 Hz, 1H), 7.42 (d, J=7.2 Hz, 1H), 7.20 (t, J=4.9 Hz, 1H), 4.64 (dd, J=11.5 Hz, 3.2 Hz, 1H), 4.27 (dd, J=12.8 Hz, 4.5 Hz, 1H), 3.11-2.96 (m, 1H), 2.68 (d, J=10.2 Hz, 1H), 1.92 (d, J=13.1 Hz, 1H), 1.75 (t, J=13.2 Hz, 2H), 1.25 (dd, J=13.3 Hz, 9.3 Hz, 1H), 0.90 (dt, J=14.5 Hz, 12.0 Hz, 1H); ¹³C NMR (DMSO-d6; 101 MHz) δ 164.5 (Cq), 158.2 (2CH), 157.7 (Cq), 151.4 (Cq), 135.2 (Cq), 133.6 (Cq), 132.9 (Cq), 128.9 (CH), 122.4 (CH), 117.7 (CH), 115.3 (CH), 57.2 (CH), 39.0 (CH₂), 30.0 (CH₂), 25.0 (CH₂), 23.0 (CH₂); HRMS: calc. for C₁₇H₁₈N₅O₂ 324.1461; found 324.1449.

### 1-(6-Methyl-pyrazin-2-yl)-3-(6-oxo-1,2,3,4,6,10b-hexahydro-pyrido[2,1-a]isoindol-10-yl)-urea (43)

The compound 43 was obtained according to the procedure A after purification by flash chromatography (CH₂Cl₂/MeOH: 99/1) as a beige solid. Yield: 82%. m.p. 142° C.; IR (ATR-Ge v cm⁻¹): 3207 (N—H amide), 2948 (Cₛₚ₃—H), 1665 (C═O amide), 1594-1487 (C═C arom), 1280 (C—N); ¹H NMR (DMSO-d6; 400 MHz) δ 10.00 (s, 1H), 9.61 (s, 1H), 8.72 (s, 1H), 8.27-8.05 (m, 2H), 7.54-7.33 (m, 2H), 4.55 (dd, J=11.5 Hz, 3.1 Hz, 1H), 4.26 (dd, J=12.7 Hz, 3.7 Hz, 1H), 3.10-2.93 (m, 1H), 2.60 (d, J=10.5 Hz, 1H), 2.46 (s, 3H), 1.89 (d, J=13.1 Hz, 1H), 1.75 (d, J=12.5 Hz, 1H), 1.62 (dd, J=26.1 Hz, 13.1 Hz, 1H), 1.25 (dd, J=17.4 Hz, 8.4 Hz, 1H), 0.88 (dd, J=23.4 Hz, 11.1 Hz, 1H); ¹³C NMR (DMSO-d6; 101 MHz) δ 164.5 (Cq), 151.7 (Cq), 150.3 (Cq), 148.3 (Cq), 137.2 (CH), 135.3 (Cq), 133.5 (Cq), 132.9 (Cq), 131.9 (CH), 128.8 (CH), 123.1 (CH), 117.8 (CH), 57.3 (CH), 39.1 (CH₂), 29.8 (CH₂), 25.0 (CH₂), 23.1 (CH₂), 20.7 (CH₃); HRMS: calc. for C₁₈H₂₀N₅O₂ 338.1617; found 338.1621.

### 1-(3-Methyl-pyrazin-2-yl)-3-(6-oxo-1,2,3,4,6,10b-hexahydro-pyrido[2,1-a]isoindol-10-yl)-urea (44)

The compound 44 was obtained according to the procedure A after purification by flash chromatography (CH₂Cl₂/MeOH: 99/1) as a white solid. Yield: 70% m.p. 228° C.; IR (ATR-Ge v cm⁻¹): 3255 (N—H amide), 2935 (Cₛₚ₃—H), 1677 (C═O amide), 1597-1481 (C═C arom), 1280 (C—N); ¹H NMR (CDCl₃, 400 MHz) δ 11.53 (s, 1H), 8.19 (d, J=2.8 Hz, 1H), 8.14 (d, J=8.0 Hz, 1H), 8.01 (d, J=2.7 Hz, 1H), 7.69-7.58 (m, 2H), 7.47 (t, J=7.8 Hz, 1H), 4.54 (dd, J=13.3 Hz, 4.8 Hz, 1H), 4.46 (dd, J=11.7 Hz, 3.5 Hz, 1H), 3.01 (td, J=13.0 Hz, 3.5 Hz, 1H), 2.74-2.57 (m, 4H), 2.03 (d, J=13.4 Hz, 1H), 1.84 (d, J=13.3 Hz, 1H), 1.77-1.63 (m, 1H), 1.52-1.35 (m, 1H), 1.10 (qd, J=13.0 Hz, 3.3 Hz, 1H); ¹³C NMR (CDCl₃, 101 MHz) δ 166.0 (Cq), 152.3 (Cq), 147.4 (Cq), 143.2 (Cq), 137.1 (CH), 136.8 (CH), 136.1 (Cq), 133.7 (Cq), 132.8 (Cq), 129.4 (CH), 124.3 (CH), 120.0 (CH), 58.5 (CH), 40.0 (CH₂), 30.5 (CH₂), 25.4 (CH₂), 24.1 (CH₂), 20.6 (CH₃). HRMS: calculated for C₁₈H₂₀N₅O₂ 338.1617; found 338.1629.

### 1-(3-Methylpyridin-2-yl)-3-(6′-oxo-3′,4′,6′,10b′-tetrahydro-1′H-spiro[1,3]dioxolane-2,2′-pyrido[2,1-i]isoindole]-10′-yl)urea (45)

The compound 45 was obtained according to the procedure A with a yield of 72%, as a white solid. m.p.>260° C.; IR (ATR-Ge v cm⁻¹): 1679 (C═O amide), 1566 (N—H amide), 1480-1415 (C═C arom), 1291 (C—N), 1135 (C—C). ¹H NMR (DMSO-d₆, 400 MHz) δₚₚₘ: 12.4 (s, 1H), 8.85 (s, 1H), 8.33 (d, J=7.5 Hz, 1H), 8.23 (d, J=7.5 Hz, 1H), 7.69 (d, J=7.0 Hz, 1H), 7.39-7.50 (m, 2H), 7.09 (dd, J=7.0 Hz, J=8.0 Hz, 1H), 4.84 (dd, J=3.3 Hz, J=11.5 Hz, 1H), 4.30 (dd, J=3.2 Hz, J=13.7 Hz, 1H), 3.81-4.07 (m, 4H), 3.17 (td, J=3.2 Hz, J=13.3 Hz, 1H), 2.62 (d, J=11.5 Hz, 1H), 3.32 (s, 3H), 1.86 (d, J=12.5 Hz, 1H), 1.53 (td, J=5.0 Hz, J=12.5 Hz, 1H), 1.35 (t, J=12.5 Hz, 1H). ¹³C NMR (DMSO-d₆, 100 MHz): 166.2 (Cq), 152.7 (Cq), 151.6 (Cq), 145.5 (CH), 143.9 (Cq), 143.3 (Cq), 137.2 (CH), 134.5 (Cq), 133.0 (Cq), 129.9 (CH), 129.2 (CH), 121.7 (CH), 112.7 (CH), 107.3 (Cq), 64.6 (CH₂), 64.3 (CH₂), 55.7 (CH), 37.8 (CH₂), 36.4 (CH₂), 34.4 (CH₂), 17.5 (CH₃). HRMS: calc. for C₂₁H₂₃N₄O₄: 395.1719; found: 395.1731.

### 1-(6-Methylpyridin-2-yl)-3-(6′-oxo-3′,4′,6′,10b′-tetrahydro-1′H-spiro[1,3]dioxolane-2,2′-pyrido[2,1-a]isoindole]-10′-yl)urea (46)

The compound 46 was obtained according to the procedure A with a yield of 75%, as a white solid. m.p. 240-242° C.; IR (ATR-Ge v cm⁻¹): 3180 (N—H amine), 1685 (C═O amide), 1584-1560-1510 (C═C arom), 1327 (C—N), 1287 (C—C), 751 (C—H arom). ¹H NMR (CDCl₃, 400 MHz) δₚₚₘ 12.14 (s, 1H), 9.16 (s, 1H), 7.99 (d, J=7.5 Hz, 1H), 7.67 (d, J=7.5 Hz, 1H), 7.47-7.59 (m, 2H), 6.82 (d, J=7.5 Hz, 1H), 6.73 (d, J=7.5 Hz, 1H), 4.92 (dd, J=2.5 Hz, J=12.5 Hz, 1H), 4.52 (dd, J=5.0 Hz, J=12.5 Hz, 1H), 3.77-3.97 (m, 4H), 3.27 (td, J=2.5 Hz, J=12.5 Hz, 1H), 2.60 (s, 3H), 2.53 (d, J=12.5 Hz, 1H), 1.79 (d, J=12.5 Hz, 1H), 1.64 (td, J=5.0 Hz, J=12.5 Hz, 1H), 1.33 (t, J=12.5 Hz, 1H). ¹³C NMR (CDCl₃, 100 MHz): 166.0 (Cq), 155.4 (Cq), 153.9 (Cq), 152.4 (Cq), 139.1 (CH), 136.1 (Cq), 133.2 (Cq), 132.8 (Cq), 129.1 (CH), 125.8 (CH), 119.7 (CH), 116.7 (CH), 109.3 (CH), 107.4 (Cq), 64.5 (CH₂), 64.4 (CH₂), 56.4 (CH), 38.1 (CH₂), 36.5 (CH₂), 34.0 (CH₂), 23.8 (CH₃). HRMS: calc. for C₂₁H₂₃N₄O₄: 395.1719; found: 395.1733.

### 1-(6-Bromopyridin-2-yl)-3-(6′-oxo-3′,4′,6′,10b′-tetrahydro-1′H-spiro[1,3]dioxolane-2,2′-pyrido[2,1-a]isoindole]-10′-yl)urea (47)

The compound 47 was obtained according to procedure A with a yield of 60%, as a white solid. m.p. 184-186° C.; IR (ATR-Ge v cm⁻¹): 2977 (N—H amine), 1685 (C═O amide), 1565-1511-1434 (C═C arom), 1326 (C—N), 1289 (C—C), 752 (C—H arom) ¹H NMR (CDCl₃, 400 MHz) δₚₚₘ 10.97 (s, 1H), 9.88 (s, 1H), 7.87 (d, J=7.5 Hz, 1H), 7.72 (d, J=7.5 Hz, 1H), 7.47-7.55 (m, 2H), 7.15 (d, J=7.5 Hz, 1H), 7.03 (d, J=10.0 Hz, 1H), 4.99 (dd, J=2.5 Hz, J=12.5 Hz, 1H), 4.53 (dd, J=2.5 Hz, J=12.5 Hz, 1H), 3.86-3.96 (m, 4H), 3.27 (td, J=2.5 Hz, J=12.5 Hz, 1H), 2.54 (d, J=15.0 Hz, 1H), 1.79 (d, J=12.5 Hz, 1H), 1.68-1.95 (m, 2H), 1.34 (t, J=12.5 Hz, 1H). ¹³C NMR (CDCl₃, 100 MHz): 165.9 (Cq), 153.4 (Cq), 152.9 (Cq), 140.7 (CH), 137.9 (Cq), 136.9 (Cq), 133.5 (Cq), 132.1 (Cq), 129.1 (CH), 126.4 (CH), 121.1 (CH), 120.4 (CH), 110.9 (CH), 107.3 (Cq), 64.7 (CH₂), 64.6 (CH₂), 56.5 (CH), 38.2 (CH₂), 36.6 (CH₂), 34.2 (CH₂). HRMS: calc. for C₂₀H₂₀N₄O₄Br: 459.0668; found 459.0685.

### 1-(4-Methoxyquinolin-2-yl)-3-(6′-oxo-3′,4′,6′,10b′-tetrahydro-1′H-spiro[1,3]dioxolane-2,2′-pyrido[2,1-a]isoindole]-10′-yl)urea (48)

The compound 48 was obtained according to procedure A with a yield of 72%, as a yellow solid. m.p. 258-260° C.; IR (ATR-Ge v cm⁻¹): 2967 (N—H amine), 1689 (C═O amide), 1621-1585-1414 (C═C arom), 1332 (C—N), 1289 (C—C), 751 (C—H arom). ¹H NMR (CDCl₃, 400 MHz) δₚₚₘ 12.51 (s, 1H), 8.95 (s, 1H), 8.23 (d, J=7.5 Hz, 1H), 8.10 (d, J=7.5 Hz, 1H), 7.95 (d, J=7.5 Hz, 1H), 7.67-7.73 (m, 2H), 7.38-7.49 (m, 2H), 6.47 (s, 1H), 5.01 (dd, J=2.5 Hz, J=12.5 Hz, 1H), 4.56 (dd, J=2.5 Hz, J=12.5 Hz, 1H), 4.02 (s, 3H), 3.57-3.79 (m, 3H), 3.27-3.39 (m, 2H), 2.72 (d, J=15.0 Hz, 1H), 1.79 (d, J=12.5 Hz, 1H), 1.70 (td, J=5.0 Hz, J=12.5 Hz, 1H), 1.38 (t, J=12.5 Hz, 1H). ¹³C NMR (CDCl₃, 100 MHz): 166.0 (Cq), 163.9 (Cq), 154.3 (Cq), 153.3 (Cq), 145.8 (Cq), 135.6 (Cq), 133.3 (Cq), 133.0 (Cq), 130.5 (CH), 128.8 (CH), 126.5 (CH), 125.2 (CH), 124.1 (CH), 121.8 (CH), 119.6 (CH), 118.8 (Cq), 107.2 (Cq), 91.5 (CH), 64.6 (CH₂), 64.5 (CH₂), 56.5 (CH), 55.7 (CH₃), 38.3 (CH₂), 36.5 (CH₂), 34.1 (CH₂). HRMS: calc. for C₂₅H₂₅N₄O₅: 461.1825; found 461.1844.

### 1-(4-Methoxyquinolin-2-yl)-3-(6′-oxo-3′,4′,6′,10b′-tetrahydro-1′H-spiro[1,3]dithiolane-2,2′-pyrido[2,1-a]isoindole]-10′-yl)urea (49)

The compound 49 was obtained according to procedure A with a yield of 80%, as a yellow solid. m.p. 250-252° C.; IR (ATR-Ge v cm⁻¹): 2975 (N—H amine), 1685 (C═O amide), 1621-1585-1414 (C═C arom), 1332 (C—N), 1289 (C—C), 751 (C—H arom). ¹H NMR (CDCl₃, 400 MHz) δₚₚₘ 12.56 (s, 1H), 10.01 (s, 1H), 8.08 (d, J=7.5 Hz, 1H), 8.00 (dd, J=7.5 Hz, J=8.0 Hz, 2H), 7.72 (d, J=7.5 Hz, 1H), 7.64 (dd, J=7.5 Hz, J=8.0 Hz, 1H), 7.49 (d, J=7.5 Hz, 1H), 7.39 (dd, J=7.5 Hz, J=8.0 Hz, 1H), 6.48 (s, 1H), 5.07 (dd, J=2.5 Hz, J=12.5 Hz, 1H), 4.58 (dd, J=2.5 Hz, J=12.5 Hz, 1H), 4.00 (s, 3H), 3.21 (td, J=5.0 Hz, J=12.5 Hz, 1H), 2.78-3.13 (m, 4H), 2.47-2.57 (m, 1H), 2.19 (d, J=12.5 Hz, 1H), 2.06 (td, J=5.0 Hz, J=12.5 Hz, 1H), 1.68 (t, J=12.5 Hz, 1H). ¹³C NMR (CDCl₃, 100 MHz): 166.0 (Cq), 163.9 (Cq), 154.3 (Cq), 153.3 (Cq), 145.8 (Cq), 136.3 (Cq), 133.4 (Cq), 132.8 (Cq), 130.4 (CH), 128.7 (CH), 127.0 (CH), 126.8 (CH), 124.2 (CH), 121.8 (CH), 120.2 (CH), 118.7 (Cq), 91.5 (CH), 65.3 (Cq), 57.7 (CH₂), 55.7 (CH₃), 45.4 (CH₂), 40.1 (CH₂), 39.0 (CH₂), 38.3 (CH₂), 38.1 (CH₂). HRMS: calc. for C₂₅H₂₅N₄O₃S₂: 493.1368; found: 493.1384.

### 1-(6-Methylpyridin-2-yl)-3-(6′-oxo-3′,4′,6′,10b′-tetrahydro-1′H-spiro[1,3]dithiolane-2,2′-pyrido[2,1-a]isoindole]-10′-yl)urea (50)

The compound 50 was obtained according to procedure A with a yield of 78%, as a white solid. m.p. 154-156° C.; IR (ATR-Ge v cm⁻¹): 2922 (N—H amine), 1689 (C═O amide), 1622-1596-1429 (C═C arom), 1355 (C—N), 1290 (C—C), 741 (C—H arom). ¹H NMR (CDCl₃, 400 MHz) δₚₚₘ 12.05 (s, 1H), 9.48 (s, 1H), 7.75 (d, J=8.0 Hz, 1H), 7.68 (d, J=8.0 Hz, 1H), 7.58-7.65 (m, 1H), 7.47 (dd, J=7.5 Hz, J=8.0 Hz, 1H), 6.84 (d, J=8.0 Hz, 2H), 4.92 (dd, J=2.5 Hz, J=12.5 Hz, 1H), 4.52 (dd, J=2.5 Hz, J=12.5 Hz, 1H), 3.14-3.29 (m, 5H), 3.88 (d, J=12.5 Hz, 1H), 2.60 (s, 3H), 2.18 (d, J=12.5 Hz, 1H), 2.03 (td, J=5.0 Hz, J=12.5 Hz, 1H), 1.61 (t, J=12.5 Hz, 1H). ¹³C NMR (CDCl₃, 100 MHz): 165.8 (Cq), 154.0 (Cq), 152.1 (Cq), 139.7 (Cq), 136.8 (CH), 133.4 (CH), 132.4 (Cq), 129.1 (2 CH), 127.0 (Cq), 120.4 (CH), 116.8 (CH), 109.7 (Cq), 65.5 (Cq), 57.8 (CH), 45.1 (CH₂), 40.5 (CH₂), 38.8 (CH₂), 38.6 (CH₂), 38.3 (CH₂), 24.8 (CH₃). HRMS: calc. for C₂₁H₂₃N₄O₂S₂: 427.1262; found 427.1278.

### 1-(5-Methylpyrazin-2-yl)-3-(6′-oxo-3′,4′,6′,10b′-tetrahydro-1′H-spiro[1,3]dithiolane-2,2′-pyrido[2,1-a]isoindole]-10′-yl)urea (51)

The compound 51 was obtained according to procedure A with a yield of 85%, as a white solid. m.p.>260° C.; IR (ATR-Ge v cm⁻¹): 2977 (N—H amine), 1688 (C═O amide), 1621-1586-1414 (C═C arom), 1332 (C—N), 1288 (C—C), 753 (C—H arom). ¹H NMR (DMSO-d₆, 400 MHz) δₚₚₘ 10.03 (s, 2H), 8.73 (s, 1H), 8.26 (s, 1H), 8.07 (d, J=7.5 Hz, 1H), 7.42-7.52 (m, 2H), 4.75 (dd, J=2.8 Hz, J=12.2 Hz, 1H), 4.32 (dd, J=2.8 Hz, J=12.2 Hz, 1H), 3.38-3.43 (m, 1H), 3.27-3.34 (m, 3H), 3.14 (t, J=12.5 Hz, 1H), 2.86 (d, J=12.5 Hz, 1H), 2.42 (s, 3H), 2.12 (d, J=12.8 Hz, 1H), 1.93 (td, J=5.0 Hz, J=12.5 Hz, 1H), 1.62 (t, J=12.5 Hz, 1H). ¹³C NMR (DMSO-d₆, 100 MHz): 164.5 (Cq), 151.7 (Cq), 146.8 (Cq), 145.8 (Cq), 139.5 (CH), 134.5 (Cq), 134.2 (CH), 133.4 (Cq), 132.6 (Cq), 128.9 (CH), 123.8 (Cq), 118.0 (CH), 65.5 (Cq), 56.8 (CH), 43.9 (CH₂), 40.4 (CH₂), 38.5 (CH₂), 38.1 (CH₂), 37.7 (CH₂), 20.0 (CH₃). HRMS: calc. for C₂₀H₂₂N₅O₂S₂ 428.1215; found 428.1229.

### 1-(4-Methoxyquinolin-2-yl)-3-(6-oxo-1,2,3,4,6,10b-hexahydropyrido[2,1-a]isoindol-10-yl)urea (52)

The compound 52 was obtained according to procedure A with a yield of 80%, as a white solid. m.p.>260° C.; IR (ATR-Ge v cm⁻¹): 1692 (C═O amide), 1588 (N—H amide), 1521-1480-1416 (C═C arom), 1286 (C—O). ¹H NMR (DMSO-d₆, 400 MHz) δₚₚₘ 11.98 (s, 1H), 10.15 (s, 1H), 8.28 (d, J=7.5 Hz, 1H), 8.05 (d, J=8.2 Hz, 1H), 7.90 (d, J=8.2 Hz, 1H), 7.77 (dd, J=7.5 Hz, J=8.2 Hz, 1H), 7.43-7.54 (m, 3H), 6.85 (s, 1H), 4.85 (dd, J=2.5 Hz, J=12.5 Hz, 1H), 4.30 (dd, J=2.8 Hz, J=12.5 Hz, 1H), 4.03 (s, 3H), 3.13 (td, J=2.8 Hz, J=12.5 Hz, 1H), 2.66 (d, J=12.5 Hz, 1H), 1.77 (d, J=12.5 Hz, 2H), 1.60-1.66 (m, 1H), 1.22-1.28 (m, 1H), 0.95 (q, J=12.5 Hz, 1H). ¹³C NMR (DMSO-d₆, 100 MHz): 165.1 (Cq), 163.6 (Cq), 154.3 (Cq), 152.8 (Cq), 146.4 (Cq), 136.2 (Cq), 134.2 (Cq), 133.6 (Cq), 131.0 (CH), 129.1 (CH), 126.4 (CH), 124.4 (CH), 124.3 (CH), 122.0 (CH), 118.7 (Cq), 118.3 (CH), 92.6 (CH), 57.9 (CH), 56.5 (CH₃), 39.6 (CH₂), 30.2 (CH₂), 25.5 (CH₂), 23.5 (CH₂). HRMS: calc. for C₂₃H₂₃N₄O₃: 403.1770; found 403.1763

### 1-(2-Methoxy-6-oxo-1,2,3,4,6,10b-hexahydropyrido[2,1-a]isoindol-10-yl)-3-(pyridin-2-yl)urea (53)

The compound 53 was obtained according to procedure A with a yield of 81%, as a yellow solid. m.p. 166-168° C.; IR (ATR-Ge v cm⁻¹): 1688 (C═O amide), 1602 (C═O ketone), 1579 (N—H amide), 1478-1418 (C═C arom), 1312 (C—N), 1291 (C—C), 751 (C—H arom); ¹H NMR (CDCl₃, 400 MHz) δₚₚₘ: 11.98 (s, 1H), 8.66 (s, 1H), 7.26 (d, J=7.5 Hz, 1H), 8.15 (d, J=8.0 Hz, 1H), 7.70 (dd, J=7.5 Hz, J=8.0 Hz, 1H), 7.64 (d, J=7.2 Hz, 1H), 7.49 (dd, J=7.5 Hz, J=8.0 Hz, 1H), 7.01 (dd, J=7.5 Hz, J=8.0 Hz, 1H), 6.90 (d, J=8.0 Hz, 1H), 4.60 (dd, J=3.2 Hz, J=12.5 Hz, 2H), 3.58-3.66 (m, 1H), 3.36 (s, 3H), 3.00-3.09 (m, 2H), 2.21 (d, J=12.5 Hz, 1H), 1.13-1.42 (m, 1H), 1.04 (t, J=12.5 Hz, 1H). ¹³C NMR (CDCl₃, 100 MHz) δₚₚₘ 166.2 (Cq), 153.5 (Cq), 152.9 (Cq), 145.7 (CH), 139.4 (CH), 135.2 (Cq), 133.4 (Cq), 133.3 (Cq), 129.5 (CH), 124.7 (CH), 119.7 (Cq), 117.9 (CH), 112.6 (CH), 77.6 (CH), 57.1 (CH), 56.1 (CH₃), 45.5 (CH₂), 35.7 (CH₂), 30.9 (CH₂). HRMS: calc. for C₁₉H₂₁N₄O₃: 353.1614; found 353.1603.

### 1-(2-Methoxy-6-oxo-1,2,3,4,6,10b-hexahydropyrido[2,1-a]isoindol-10-yl)-3-(pyridin-2-yl)urea (54)

The compound 54 was obtained according to procedure A with a yield of 78%, as a yellow solid. m.p. 192-194° C.; IR (ATR-Ge v cm⁻¹): 1684 (C═O amide), 1595 (C═O ketone), 1557 (N—H amide), 1486-1421 (C═C arom), 1306 (C—N), 1084 (C—C), 753 (C—H arom). ¹H NMR (DMSO-d₆, 400 MHz) δₚₚₘ 10.08 (s, 1H), 9.79 (s, 1H), 8.95 (s, 1H), 8.30-8.35 (m, 2H), 8.14 (d, J=8.0 Hz, 1H), 7.48 (dd, J=7.8 Hz, J=8.0 Hz, 1H), 7.41 (d, J=8.0 Hz, 1H), 4.66 (dd, J=3.2 Hz, J=12.5 Hz, 1H), 4.28 (dd, J=4.0 Hz, J=13.5 Hz, 1H), 3.64-3.69 (m, 1H), 3.28 (s, 3H), 3.08 (td, J=3.2 Hz, J=12.5 Hz, 1H), 2.91 (d, J=13.5 Hz, 1H), 2.13 (d, J=12.5 Hz, 1H), 1.07-1.18 (m, 1H), 0.74 (q, J=12.5 Hz, 1H). ¹³C NMR (DMSO-d₆, 100 MHz) δₚₚₘ: 164.5 (Cq), 151.6 (Cq), 149.1 (Cq), 141.1 (CH), 137.9 (CH), 135.4 (CH), 134.5 (Cq), 133.5 (Cq), 132.8 (Cq), 129.0 (CH), 123.0 (CH), 117.9 (CH), 76.2 (CH), 55.8 (CH), 55.1 (CH₃), 36.4 (CH₂), 35.2 (CH₂), 30.6 (CH₂). HRMS: calc. for C₁₈H₁₉N₅O₃Na: 376.1386; found 376.1370.

### 1-(2-Methoxy-6-oxo-1,2,3,4,6,10b-hexahydropyrido[2,1-a]isoindol-10-yl)-3-(4-methoxyquinolin-2-yl)urea (55)

The compound 55 was obtained according to procedure A with a yield of 80%, as a white solid. m.p.>260° C. IR (ATR-Ge v cm⁻¹): 1694 (C═O amide), 1610 (C═O ketone), 1556 (N—H amide), 1479-1416 (C═C arom), 1338 (C—N), 1295 (C—C), 759 (C—H arom). ¹H NMR (DMSO-d₆, 400 MHz) δₚₚₘ 11.99 (s, 1H), 10.15 (s, 1H), 8.30 (d, J=8.0 Hz, 1H), 8.02 (d, J=8.0 Hz, 1H), 7.92 (d, J=8.0 Hz, 1H), 7.77 (dd, J=7.5 Hz, J=8.0 Hz, 1H), 7.41-7.51 (m, 3H), 6.84 (s, 1H), 4.92 (dd, J=3.2 Hz, J=12.0 Hz, 1H), 4.30 (dd, J=4.0 Hz, J=13.5 Hz, 1H), 4.01 (s, 3H), 3.59 (d, J=12.0 Hz, 1H), 3.15 (td, J=3.2 Hz, J=12.5 Hz, 1H), 2.91 (d, J=13.5 Hz, 1H), 2.82 (s, 3H), 2.09 (d, J=12.5 Hz, 1H), 1.05-1.15 (m, 1H), 0.76 (q, J=12.5 Hz, 1H). ¹³C NMR (DMSO-d₆, 100 MHz) δₚₚₘ 164.6 (Cq), 162.9 (Cq), 153.7 (Cq), 152.3 (Cq), 145.6 (Cq), 134.3 (Cq), 133.7 (Cq), 132.8 (Cq), 130.7 (CH), 128.9 (CH), 126.3 (CH), 124.1 (CH), 123.5 (Cq), 121.5 (CH), 118.0 (Cq), 117.8 (CH), 91.9 (CH), 76.3 (CH), 56.0 (CH), 55.8 (CH₃), 54.7 (CH₃), 36.4 (CH₂), 34.4 (CH₂), 30.7 (CH₂). HRMS: calc. for C₂₄H₂₅N₄O₄ 433.1876; found 433.1884.

### 1-(2-Methoxy-6-oxo-1,2,3,4,6,10b-hexahydropyrido[2,1-a]isoindol-10-yl)-3-(6-methylpyri-din-2-yl)urea (56)

The compound 56 was obtained according to the procedure A with a yield of 75% in the form of a white solid. 167-169° C. IR (ATR-Ge v cm⁻¹): 1689 (C═O amide), 1589 (C═O ketone), 1556 (N—H amide), 1436 (C═C arom), 1283 (C—N), 751 (C—H arom). ¹H NMR (CDCl₃, 400 MHz) δₚₚₘ 11.98 (s, 1H), 8.93 (s, 1H), 7.99 (d, J=8.0 Hz, 1H), 7.66 (d, J=8.0 Hz, 1H), 7.55 (dd, J=7.5 Hz, J=8.0 Hz, 1H), 7.49 (dd, J=7.5 Hz, J=8.0 Hz, 1H), 6.83 (d, J=7.5 Hz, 1H), 6.70 (d, J=8.0 Hz, 1H), 4.69 (dd, J=3.2 Hz, J=12.0 Hz, 1H), 4.58 (dd, J=4.5 Hz, J=13.5 Hz, 1H), 3.52-3.59 (m, 1H), 3.30 (s, 3H), 3.02 (td, J=3.2 Hz, J=12.5 Hz, 1H), 2.93 (d, J=12.5 Hz, 1H), 2.56 (s, 3H), 2.19 (d, J=12.5 Hz, 1H), 1.27-1.38 (m, 1H), 1.03 (q, J=12.5 Hz, 1H). ¹³C NMR (CDCl₃, 100 MHz) δₚₚₘ 166.2 (Cq), 155.1 (Cq), 153.9 (Cq), 152.5 (Cq), 139.4 (CH), 136.0 (Cq), 133.5 (Cq), 133.1 (Cq), 129.4 (CH), 125.9 (CH), 120.1 (CH), 117.1 (CH), 109.5 (CH), 77.4 (CH), 57.3 (CH), 56.0 (CH₃), 37.3 (CH₂), 35.3 (CH₂), 30.8 (CH₂), 24.6 (CH₃). HRMS: calc. for C₂₀H₂₃N₄O₃: 367.1770; found 367.1776.

### 1-(2-Methoxy-6-oxo-1,2,3,4,6,10b-hexahydropyrido[2,1-a]isoindol-10-yl)-3-(3-methylpyridin-2-yl)urea (57)

The compound 57 was obtained according to procedure A with a yield of 73% in the form of a yellow solid. 222-224° C. IR (ATR-Ge v cm⁻¹): 1692 (C═O amide), 1592 (C═O ketone), 1554 (N—H amide), 1484-1421 (C═C arom), 1298 (C—N), 1189 (C—C), 751 (C—H arom). ¹H NMR (CDCl₃, 400 MHz) δₚₚₘ: 12.24 (s, 1H), 8.13-8.16 (m, 2H), 7.61 (d, J=7.5 Hz, 1H), 7.53 (d, J=7.5 Hz, 1H), 7.46 (dd, J=7.5 Hz, J=8.0 Hz, 1H), 7.10 (s, 1H), 6.95 (dd, J=7.5 Hz, J=8.0 Hz, 1H), 4.58 (dd, J=3.2 Hz, J=12.0 Hz, 2H), 3.60-3.65 (m, 1H), 3.36 (s, 3H), 3.03 (td, J=3.2 Hz, J=12.5 Hz, 2H), 2.31 (s, 3H), 2.20 (d, J=12.5 Hz, 1H), 1.30-1.40 (m, 1H), 1.02 (q, J=12.5 Hz, 1H). ¹³C NMR (CDCl₃, 100 MHz) δₚₚₘ 166.2 (Cq), 152.7 (Cq), 151.2 (Cq), 143.2 (CH), 139.9 (CH), 135.1 (Cq), 133.3 (Cq), 133.2 (Cq), 129.5 (CH), 124.6 (CH), 119.8 (Cq), 119.6 (CH), 117.9 (CH), 77.6 (CH), 57.1 (CH), 56.1 (CH₃), 37.2 (CH₂), 35.7 (CH₂), 30.9 (CH₂), 17.1 (CH₃). HRMS: calc. for C₂₀H₂₃N₄O₃: 367.1770; found 367.1778.

### 1-(2-methoxy-6-oxo-1,2,3,4,6,10b-hexahydropyrido[2,1-a]isoindol-10-yl)-3-(4-methylpyridin-2-yl)urea (58)

The compound was obtained according to procedure A with a yield of 76% as a yellow solid. 218-220° C. IR (ATR-Ge v cm⁻¹): 1690 (C═O amide), 1619 (C═O ketone), 1573 (N—H amide), 1481-1439 (C═C arom), 1310 (C—N), 1293 (C—C), 752 (C—H arom). ¹H NMR (DMSO-d₆, 400 MHz) δₚₚₘ 11.41 (s, 1H), 9.88 (s, 1H), 8.26 (d, J=8.0 Hz, 1H), 8.18 (d, J=7.5 Hz, 1H), 7.45 (dd, J=7.5 Hz, J=8.0 Hz, 1H), 7.36 (d, J=7.5 Hz, 1H), 7.08 (s, 1H), 6.92 (d, J=7.5 Hz, 1H), 4.69 (dd, J=3.2 Hz, J=12.0 Hz, 1H), 4.28 (dd, J=3.2 Hz, J=12.0 Hz, 1H), 3.68-3.77 (m, 1H), 3.24 (s, 3H), 3.08 (td, J=3.2 Hz, J=12.5 Hz, 2H), 2.29 (s, 3H), 2.13 (d, J=12.0 Hz, 1H), 1.07-1.18 (m, 1H), 0.75 (q, J=12.0 Hz, 1H). ¹³C NMR (DMSO-d₆, 100 MHz) δₚₚₘ 164.7 (Cq), 152.9 (Cq), 152.2 (Cq), 150.0 (Cq), 145.6 (CH), 134.0 (Cq), 133.9 (Cq), 132.7 (Cq), 129.1 (CH), 122.3 (CH), 118.9 (CH), 117.4 (CH), 112.1 (CH), 76.3 (CH), 55.8 (CH), 55.2 (CH₃), 36.5 (CH₂), 35.2 (CH₂), 30.7 (CH₂), 20.8 (CH₃). HRMS: calc. for C₂₀H₂₃N₄O₃: 367.1770; found 367.1761.

1-(2-methoxy-6-oxo-1,2,3,4,6,10b-hexahydropyrido[2,1-a]isoindol-10-yl)-3-(5-methylpyrazin-2-yl)urea (59).

The compound 59 was obtained according to procedure A with a yield of 76% in the form of a yellow solid. m.p. 140-142° C. IR (ATR-Ge v cm⁻¹): 1688 (C═O amide), 1598 (C═O ketone), 1554 (N—H amide), 1484-1438 (C═C arom), 1344 (C—N), 1291 (C—C), 753 (C—H arom). ¹H NMR (CDCl₃, 400 MHz) δₚₚₘ 11.16 (s, 1H), 9.80 (s, 1H), 8.42 (s, 1H), 8.11 (d, J=8.0 Hz, 1H), 8.02 (s, 1H), 7.66 (d, J=7.5 Hz, 1H), 7.49 (dd, J=7.5 Hz, J=8.0 Hz, 1H), 4.53-4.62 (m, 2H), 3.58-3.64 (m, 1H), 3.38 (s, 3H), 2.96-3.06 (m, 2H), 2.54 (s, 3H), 2.22 (d, J=12.5 Hz, 1H), 1.30-1.40 (m, 1H), 1.06 (q, J=12.0 Hz, 1H). ¹³C NMR (CDCl₃, 100 MHz) δₚₚₘ 165.8 (Cq), 153.4 (Cq), 146.7 (Cq), 146.6 (Cq), 137.7 (CH), 135.1 (CH), 135.0 (Cq), 133.3 (Cq), 132.6 (Cq), 129.5 (CH), 124.6 (CH), 120.0 (CH), 77.3 (CH), 56.9 (CH), 55.9 (CH₃), 37.0 (CH₂), 35.7 (CH₂), 30.5 (CH₂), 20.6 (CH₃). HRMS: calc. for C₁₉H₂₂N₅O₃: 368.1723; found 368.1722.

### 1-(2-methoxy-6-oxo-1,2,3,4,6,10b-hexahydropyrido[2,1-a]isoindol-10-yl)-3-(1-methyl-1H-pyrazol-3-yl)urea (60)

The compound 60 was obtained according to procedure A with a yield of 81% as a yellow solid 196-198° C.; IR (ATR-Ge v cm⁻¹): 1686 (C═O amide), 1625 (C═O ketone), 1538 (N—H amide), 1482-1421 (C═C arom), 1316 (C—N), 1284 (C—C), 751 (C—H arom). ¹H NMR (CDCl₃, 400 MHz) δₚₚₘ 10.06 (s, 1H), 8.17 (s, 1H), 8.05 (d, J=8.0 Hz, 1H), 7.62 (d, J=7.5 Hz, 1H), 7.45 (dd, J=7.5 Hz, J=8.0 Hz, 1H), 7.27 (s, 1H), 5.85 (s, 1H), 4.56-4.60 (m, 2H), 3.87 (s, 3H), 3.52-3.60 (m, 1H), 3.36 (s, 3H), 3.97-3.08 (m, 2H), 2.21 (d, J=12.5 Hz, 1H), 1.27-1.38 (m, 1H), 1.06 (q, J=12.5 Hz, 1H). ¹³C NMR (CDCl₃, 100 MHz) δₚₚₘ 166.0 (Cq), 152.8 (Cq), 148.2 (Cq), 135.2 (Cq), 133.3 (Cq), 133.2 (Cq), 131.4 (CH), 129.2 (CH), 125.0 (CH), 119.5 (CH), 94.1 (Cq), 77.2 (CH), 56.9 (CH), 55.7 (CH₃), 38.7 (CH₃), 37.0 (CH₂), 35.7 (CH₂), 30.3 (CH₂). HRMS: calc. for C₁₈H₂₁N₅O₃Na: 378.1542; found 378.1542.

### 1-(5-(methoxymethoxy)quinolin-2-yl)-3-(6′-oxo-3′,4′,6′,10b′-tetrahydro-1′H-spiro[[1,3]dioxolane-2,2′-pyrido[2,1-a]isoindole]-10′-yl)urea (61)

The compound 61 was obtained according to procedure A with a yield of 78% in the form of a white solid. 192-194° C. IR (ATR-Ge v cm⁻¹): 1690 (C═O amide), 1601 (C═O ketone), 1525 (N—H amide), 1422 (C═C arom), 1332 (C—N), 1298 (C—C), 778 (C—H arom). ¹H NMR (CDCl₃, 400 MHz) δₚₚₘ 12.44 (s, 1H), 10.26 (s, 1H), 8.28 (d, J=8.0 Hz, 1H), 8.03 (d, J=8.0 Hz, 1H), 7.91 (d, J=8.0 Hz, 1H), 7.58-7.65 (m, 2H), 7.41 (dd, J=7.6 Hz, J=8.0 Hz, 1H), 7.33 (dd, J=7.6 Hz, J=7.6 Hz, 1H), 6.73 (s, 1H), 5.34-5.39 (m, 2H), 4.92 (dd, J=4.0 Hz, J=12.8 Hz, 1H), 4.47 (dd, J=4.8 Hz, J=13.6 Hz, 1H), 3.50-3.68 (m, 3H), 3.49 (s, 3H), 3.21-3.27 (m, 2H), 2.63 (d, J=11.6 Hz, 1H), 1.70 (d, J=13.6 Hz, 1H), 1.61 (td, J=4.8 Hz, 12.8 Hz, 1H), 1.27 (t, J=12.8 Hz, 1H). ¹³C NMR (CDCl₃, 100 MHz) δₚₚₘ 166.1 (Cq), 161.3 (Cq), 154.4 (Cq), 153.4 (Cq), 146.0 (Cq), 135.3 (Cq), 133.1 (2 Cq), 130.4 (CH), 129.0 (2 CH), 126.7 (CH), 125.1 (CH), 124.1 (CH), 121.8 (CH), 119.3 (CH), 118.8 (Cq), 107.2 (Cq), 94.6 (CH₂), 64.6 (CH₂), 64.5 (CH₂), 56.9 (CH₃), 56.5 (CH), 38.3 (CH₂), 36.5 (CH₂), 34.3 (CH₂). MS (IS) m/z: 491.0 [M+H]⁺.

2.2. General Procedure B

Synthesis of Derivatives of Thioureas (62)-(74) from Isothiocyanates (11) or (12).

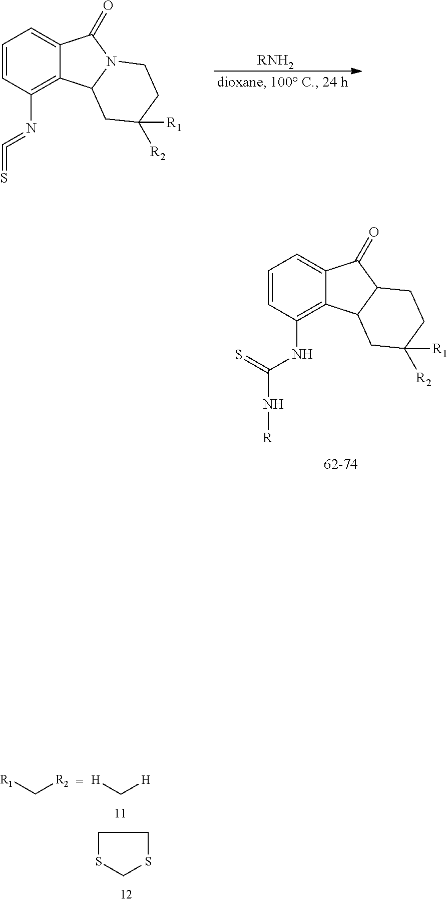

In a 25 mL flask, the isothiocyanate (0.6 mmol, 11 or 12) is dissolved in 10 mL of an anhydrous dioxane and then the amine (0.6 mmol, 1 equ.) is added. The reaction mixture is heated to 100° C. with stirring for 24 hours and then cooled in order to remove the solvent under reduced pressure. The reaction raw product is purified by chromatography on silica gel in order to obtain the expected thioureas.

### 1-(6-Oxo-1,2,3,4,6,10b-hexahydropyrido[2,1-a]isoindol-10-yl)-3-(pyridin-2-yl)thiourea (62)

The compound 62 is obtained as a white solid according to the general procedure B with a yield of 90% after purification on silica gel (CH₂Cl₂/MeOH: 99/1). MP: 195° C.; IR (ATR-Ge v cm⁻¹): 3223 (N—H amine), 1679 (C═O amide), 1597-1565-1526 (C═C arom), 1346 (C—N), 1286 (C—C), 776 (C—H arom); ¹H NMR (CDCl₃; 400 MHz) (δ 13.63 (s, 1H), 9.67 (s, 1H), 8.21 (dd, J=1.2 Hz, J=5.1 Hz, 1H), 7.85 (dd, J=7.6 Hz, J=15.5 Hz, 2H), 7.76-7.68 (m, 1H), 7.53 (t, J=7.7 Hz, 1H), 7.10-7.00 (m, 2H), 4.61 (dd, J=3.6 Hz, J=11.7 Hz, 1H), 4.52 (dd, J=4.1 Hz, J=13.3 Hz, 1H), 2.99 (td, J=4.1 Hz, J=13.0 Hz, 1H), 2.47 (dd, J=3.1 Hz, J=12.9 Hz, 1H), 1.92 (d, J=13.5 Hz, 1H), 1.79 (d, J=13.1 Hz, 1H), 1.68-1.53 (m, 1H), 1.34-1.46 (m, 1H), 1.22-1.09 (m, 1H); ¹³C NMR (CDCl₃; 101 MHz) (δ 179.7 (Cq), 165.8 (Cq), 153.3 (Cq), 145.6 (CH), 141.0 (Cq), 139.5 (CH), 134.1 (Cq), 133.7 (Cq), 129.7 (CH), 129.0 (CH), 122.6 (CH), 118.8 (CH), 112.9 (CH), 58.8 (CH), 39.9 (CH₂), 30.6 (CH₂), 25.4 (CH₂), 23.9 (CH₂); MS (IS) m/z: 339.5 [M+H]⁺, 677.5 [2M+H]⁺.

### 1-(6-Oxo-1,2,3,4,6,10b-hexahydropyrido[2,1-a]isoindol-10-yl)-3-(pyrazin-2-yl)thiourea (63)

The compound 63 is obtained as a pale brown solid according to the general procedure B with a yield of 62% after purification on silica gel (CH₂Cl₂/MeOH: 99/1). MP: 204° C.; IR (ATR-Ge v cm⁻¹): 3010 (N—H amine), 1686 (C═O amide), 1591-1560-1514 (C═C arom), 1416 (C—N), 1136 (C—C); ¹H NMR (DMSO-d6; 400 MHz) (δ 12.80 (s, 1H), 11.40 (s, 1H), 8.68 (d, J=1.0 Hz, 1H), 8.40-8.29 (m, 2H), 7.93 (d, J=7.7 Hz, 1H), 7.62 (d, J=6.9 Hz, 1H), 7.54 (t, J=7.7 Hz, 1H), 4.65 (dd, J=3.5 Hz, J=11.6 Hz, 1H), 4.24 (dd, J=3.7 Hz, J=13.0 Hz, 1H), 3.00 (td, J=3.7 Hz, J=12.9 Hz, 1H), 2.34 (dd, J=3.0 Hz, J=12.7 Hz, 1H), 1.82 (d, J=12.8 Hz, 1H), 1.73 (d, J=13.0 Hz, 1H), 1.58 (q, J=13.1 Hz, 1H), 1.31-1.19 (m, 1H), 0.97 (qd, J=3.0 Hz, J=12.7 Hz, 1H); ¹³C NMR (DMSO-d6; 101 MHz) (δ 179.3 (Cq), 164.3 (Cq), 149.3 (Cq), 140.7 (Cq), 139.4 (CH), 138.1 (CH), 136.5 (CH), 133.8 (Cq), 133.2 (Cq), 129.3 (CH), 128.6 (CH), 121.1 (CH), 57.7 (CH), 39.0 (CH₂), 30.1 (CH₂), 24.9 (CH₂), 23.0 (CH₂); MS (IS) m/z: 340.5 [M+H]⁺, 679.5 [2M+H]⁺.

### 1-(6-Oxo-1,2,3,4,6,10b-hexahydropyrido[2,1-a]isoindol-10-yl)-3-(1H-pyrazol-3-yl)thiourea (64)

The compound 64 is obtained as a beige solid according to the general procedure B with a yield of 56% after purification on silica gel (CH₂Cl₂/MeOH: 99/1). MP: 208° C.; IR (ATR-Ge v cm⁻¹): 3190 (NH amine), 1659 (C═O amide), 1579-1522-1454-1424 (C═C arom), 1258-1016 (C—C), 762 (C—H arom); ¹H NMR (DMSO-d6; 400 MHz) (δ 12.70 (s, 1H), 11.64 (s, 1H), 10.91 (s, 1H), 7.93 (t, J=8.0 Hz, 1H), 7.76 (d, J=2.3 Hz, 1H), 7.59 (d, J=6.8 Hz, 1H), 7.51 (t, J=7.7 Hz, 1H), 6.05 (s, 1H), 4.60 (dd, J=3.5 Hz, J=11.6 Hz, 1H), 4.24 (dd, J=3.8 Hz, J=13.0 Hz, 1H), 2.98 (td, J=3.8 Hz, J=12.9 Hz, 1H), 2.39 (d, J=12.4 Hz, 1H), 1.82 (d, J=13.4 Hz, 1H), 1.73 (d, J=12.7 Hz, 1H), 1.60 (q, J=13.1 Hz, 1H), 1.31-1.18 (m, 1H), 0.95 (qd, J=3.2 Hz, J=12.7 Hz, 1H); ¹³C NMR (DMSO-d6; 101 MHz) (δ 164.7 (Cq), 149.4 (Cq), 140.8 (Cq), 140.4 (Cq), 134.3 (Cq), 133.2 (Cq), 130.0 (CH), 129.6 (CH), 128.7 (CH), 121.0 (CH), 94.4 (CH), 58.0 (CH), 39.2 (CH₂), 30.2 (CH₂), 25.1 (CH₂), 23.0 (CH₂); MS (IS) m/z: 328.5 [M+H]⁺.

### 1-(6-Oxo-1,2,3,4,6,10b-hexahydropyrido[2,1-a]isoindol-10-yl)-3-(pyrimidin-2-yl)thiourea (65)

The compound 65 is obtained as a beige solid according to the general procedure B with a yield of 48% after purification on silica gel (CH₂Cl₂/MeOH: 99/1). MP: 212° C.; IR (ATR-Ge v cm⁻¹): 3145 (N—H amine), 1698 (C═O amide), 1580-1545-1522 (C═C arom), 1406 (C—N), 1198-1158 (C—C); ¹H NMR (DMSO-d6; 400 MHz) (δ 13.15 (s, 1H), 11.30 (s, 1H), 8.74 (d, J=4.9 Hz, 2H), 7.93 (d, J=7.7 Hz, 1H), 7.62 (d, J=7.0 Hz, 1H), 7.54 (t, J=7.7 Hz, 1H), 7.25 (t, J=4.9 Hz, 1H), 4.65 (dd, J=3.5 Hz, J=11.6 Hz, 1H), 4.24 (dd, J=3.7 Hz, J=12.8 Hz, 1H), 3.00 (td, J=3.7 Hz, J=12.8 Hz, 1H), 2.33 (dd, J=3.0 Hz, J=12.6 Hz, 1H), 1.82 (d, J=13.2 Hz, 1H), 1.73 (d, J=13.0 Hz, 1H), 1.58 (q, J=13.0 Hz, 1H), 1.30-1.14 (m, 1H), 0.97 (qd, J=3.0 Hz, J=12.9 Hz, 1H); ¹³C NMR (DMSO-d6; 101 MHz) δ 179.1 (Cq), 164.3 (Cq), 158.2 (2×CH), 157.4 (Cq), 140.6 (Cq), 134.0 (Cq), 133.1 (Cq), 129.2 (CH), 128.5 (CH), 121.4 (CH), 116.1 (CH), 57.6 (CH), 39.0 (CH₂), 30.1 (CH₂), 24.9 (CH₂), 22.9 (CH₂); MS (IS) m/z: 340.5 [M+H]⁺, 679.5 [2M+H]⁺.

### 1-(5-Bromopyridin-2-yl)-3-(6-oxo-1,2,3,4,6,10b-hexahydropyrido[2,1-a]isoindol-10-yl)thiourea (66)

The compound 66 is obtained as a white solid according to the general procedure B with a yield of 75% after purification on silica gel (CH₂Cl₂/MeOH: 99/1). F>260° C.; IR (ATR-Ge v cm⁻¹): 3286 (NH amine), 1671 (C═O amide), 1592-1543-1510-1468 (C═C arom), 1170 (C—N), 662 (C—Br); ¹H NMR (DMSO-d6; 400 MHz) δ 13.21 (s, 1H), 11.22 (s, 1H), 8.47 (d, J=2.2 Hz, 1H), 8.09 (dd, J=2.4 Hz, J=8.9 Hz, 1H), 7.99 (d, J=7.7 Hz, 1H), 7.60 (d, J=7.3 Hz, 1H), 7.53 (t, J=7.6 Hz, 1H), 7.27 (d, J=8.9 Hz, 1H), 4.66 (dd, J=3.2 Hz, J=11.6 Hz, 1H), 4.24 (dd, J=4.0 Hz, J=12.0 Hz, 1H), 3.02 (td, J=4.0 Hz, J=12.0 Hz, 1H), 2.32 (d, J=9.9 Hz, 1H), 1.82 (d, J=13.2 Hz, 1H), 1.73 (d, J=12.7 Hz, 1H), 1.59 (q, J=13.3 Hz, 1H), 1.30-1.17 (m, 1H), 1.03-0.90 (m, 1H); ¹³C NMR (DMSO-d6; 101 MHz) δ 178.7 (Cq), 164.3 (Cq), 152.1 (Cq), 146.0 (CH), 141.9 (CH), 140.3 (Cq), 133.9 (Cq), 133.1 (Cq), 128.9 (CH), 128.4 (CH), 120.8 (CH), 114.8 (CH), 112.5 (Cq), 57.5 (CH), 38.9 (CH₂), 30.0 (CH₂), 24.9 (CH₂), 22.9 (CH₂); MS (IS) m/z: 418.5 [M+H]⁺.

### 1-(6-Bromopyridin-2-yl)-3-(6-oxo-1,2,3,4,6,10b-hexahydropyrido[2,1-a]isoindol-10-yl)thiourea (67)

The compound 67 is obtained as a beige solid according to the general procedure B with a yield of 68% after purification on silica gel (CH₂Cl₂/MeOH: 99/1). MP: 195° C.; IR (ATR-Ge v cm⁻¹): 3225 (N—H amine), 1678 (C═O amide), 1578-1447-1433-1409 (C═C arom), 1148-1127 (C—C), 679 (C—Br); ¹H NMR (DMSO-d6; 400 MHz) δ 12.47 (s, 1H), 11.29 (s, 1H), 7.97 (d, J=7.7 Hz, 1H), 7.80 (t, J=8.0 Hz, 1H), 7.62 (d, J=7.0 Hz, 1H), 7.54 (t, J=7.7 Hz, 1H), 7.39 (d, J=7.6 Hz, 1H), 7.34 (d, J=8.2 Hz, 1H), 4.68 (dd, J=3.6 Hz, J=11.7 Hz, 1H), 4.24 (dd, J=3.7 Hz, J=12.9 Hz, 1H), 3.00 (td, J=3.7 Hz, J=12.9 Hz, 1H), 2.45 (dd, J=3.1 Hz, J=12.8 Hz, 1H), 1.85 (d, J=13.0 Hz, 1H), 1.74 (d, J=12.7 Hz, 1H), 1.70-1.55 (m, 1H), 1.32-1.15 (m, 1H), 1.02 (qd, J=3.1 Hz, J=12.6 Hz, 1H); ¹³C NMR (DMSO-d6; 101 MHz) δ 178.7 (Cq), 164.2 (Cq), 153.0 (Cq), 142.0 (CH), 140.0 (Cq), 136.5 (Cq), 133.5 (Cq), 133.2 (Cq), 129.5 (CH), 128.4 (CH), 122.0 (CH), 121.0 (CH), 112.0 (CH), 57.4 (CH), 39.0 (CH₂), 30.2 (CH₂), 24.9 (CH₂), 22.9 (CH₂); MS (IS) m/z: 418.5 [M+H]⁺.

### 1,3-Bis(6′-oxo-3′,4′,6′,10b′-tetrahydro-1′H-spiro[[1,3]dithiolane-2,2′-pyrido[2,1-a]isoindole]-10′-yl)thiourea (68)

The compound 68 is obtained as a white solid according to the general procedure B with a yield of 56% after purification on silica gel (CH₂Cl₂/MeOH: 99/1). MP: 188° C.; IR (ATR-Ge v cm⁻¹): 3195 (N—H amine), 1687 (C═O amide), 1521-1487-1454-1429 (C═C arom), 1204 (C—C), 762 (C—H arom); ¹H NMR (DMSO-d6; 250 MHz) δ 9.89 (s, 1H), 9.76 (s, 1H), 7.74-7.40 (m, 6H), 4.78 (d, J=11.3 Hz, 2H), 4.31 (d, J=13.3 Hz, 2H), 3.5-3.35 (m, 8H), 3.23-3.01 (m, 2H), 2.82-2.62 (m, 2H), 2.14 (d, J=12.5 Hz, 2H), 1.97 (td, J=4.4 Hz, J=12.6 Hz, 2H), 1.76-1.55 (m, 2H); ¹³C NMR (DMSO-d6; 101 MHz) δ 181.1 (Cq), 180.9 (Cq), 164.5 (2×Cq), 141.4 (Cq), 140.8 (Cq), 134.3 (Cq), 134.8 (Cq), 133.1 (Cq), 131.3 (CH), 130.5 (CH), 129.1 (2×CH), 121.6 (CH), 121.3 (CH), 65.6 (2×Cq), 57.4 (CH), 57.2 (CH), 44.86 (CH₂), 44.6 (CH₂), 40.0 (2×CH₂), 38.5 (2×CH₂), 38.4 (CH₂), 38.3 (CH₂), 37.9 (CH₂), 37.8 (CH₂); MS (IS) m/z: 627.5 [M+H]⁺, 649.0 [M+Na]⁺.

### 1-(6′-Oxo-3′,4′,6′,10b′-tetrahydro-1′H-spiro[[1,3]dithiolane-2,2′-pyrido[2,1-a]isoindole]-10′-yl)-3-(pyrazin-2-yl)thiourea (69)

The compound 69 is obtained as a yellow solid according to general procedure B with a yield of 62% after purification on silica gel (CH₂Cl₂/MeOH: 99/1). MP: 128° C.; IR (ATR-Ge v cm⁻¹): 3203 (N—H amine), 1686 (C═O amide), 1599-1567-1514-1484 (C═C arom), 1403 (C—N), 1140 (C—C); ¹H NMR (CDCl₃; 400 MHz) δ 12.94 (s, 1H), 9.79 (s, 1H), 8.57 (d, J=1.1 Hz, 1H), 8.34 (d, J=2.8 Hz, 1H), 8.30 (dd, J=1.4 Hz, J=2.8 Hz, 1H), 8.12 (d, J=7.8 Hz, 1H), 7.82 (d, J=7.4 Hz, 1H), 7.55 (t, J=7.7 Hz, 1H), 4.86 (dd, J=3.4 Hz, J=11.7 Hz, 1H), 4.64-4.53 (m, 1H), 3.35-3.16 (m, 4H), 3.16-3.08 (m, 1H), 2.83 (m, 1H), 2.23-2.16 (m, 1H), 2.05 (td, J=5.0 Hz, J=12.8 Hz, 1H), 1.80 (t, J=12.0 Hz, 1H); ¹³C NMR (CDCl₃; 101 MHz) δ 179.6 (Cq), 165.8 (Cq), 149.1 (Cq), 139.2 (CH), 138.7 (Cq), 138.6 (CH), 136.3 (CH), 133.7 (Cq), 133.2 (Cq), 129.4 (CH), 129.3 (CH), 122.7 (CH), 65.6 (Cq), 57.9 (CH), 45.6 (CH₂), 41.3 (CH₂), 39.3 (CH₂), 38.8 (CH₂), 38.5 (CH₂); MS (IS) m/z: 430.0 [M+H]⁺

### 1-(6′-Oxo-3′,4′,6′,10b′-tetrahydro-1′H-spiro[[1,3]dithiolane-2,2′-pyrido[2,1-a]isoindole]-10′-yl)-3-(pyridin-2-yl)thiourea (70)

The compound 70 is obtained as a beige solid according to general procedure B with a yield of 45% after purification on silica gel (CH₂Cl₂/MeOH: 99/1). MP: 142° C.; IR (ATR-Ge v cm⁻¹): 3226 (N—H amine), 1677 (C═O amide), 1598-1530-1475-1421 (C═C arom), 1144-1000 (C—C); ¹H NMR (CDCl₃; 400 MHz) δ 13.73 (s, 1H), 8.92 (s, 1H), 8.38 (dd, J=1.2, J=5.1 vHz, 1H), 8.17 (d, J=7.9 Hz, 1H), 7.81-7.71 (m, 2H), 7.54 (t, J=7.8 Hz, 1H), 7.06 (dd, J=5.2 Hz, J=6.7 Hz, 1H), 6.90 (d, J=8.3 Hz, 1H), 4.88 (dd, J=3.5 Hz, J=11.6 Hz, 1H), 4.60-4.50 (m, 1H), 3.47-3.12 (m, 4H), 3.09-3.00 (m, 1H), 2.91 (m, 1H), 2.20 (d, J=13.4 Hz, 1H), 2.09-2.01 (m, 1H), 1.79-1.73 (m, 1H); ¹³C NMR (CDCl₃; 101 MHz) δ 179.5 (Cq), 165.9 (Cq), 153.1 (Cq), 146.2 (CH), 139.6 (CH), 138.6 (Cq), 133.7 (Cq), 133.7 (Cq), 129.3 (CH), 129.1 (CH), 122.3 (CH), 118.7 (CH), 112.5 (CH), 65.7 (Cq), 57.8 (CH), 45.7 (CH₂), 41.1 (CH₂), 39.1 (CH₂), 38.9 (CH₂), 38.5 (CH₂); MS (IS) m/z: 429.0 [M+H]⁺

### 1-(6′-Oxo-3′,4′,6′,10b′-tetrahydro-1′H-spiro[[1,3]dithiolane-2,2′-pyrido[2,1-a]isoindole]-10′-yl)-3-(1H-pyrazol-3-yl)thiourea (71)

The compound 71 is obtained as a white solid according to general procedure B with a yield of 55% after purification on silica gel (CH₂Cl₂/MeOH: 99/1). MP: 185° C.; IR (ATR-Ge v cm⁻¹): 3190 (NH amine), 1669 (C═O amide), 1574, 1525-1485 (C═C arom), 1199 (C—C), 999 (C—N), 755 (C—H arom); ¹H NMR (DMSO-d6; 400 MHz) δ 12.64 (s, 1H), 11.54 (s, 1H), 10.92 (s, 1H), 7.80 (d, J=7.7 Hz, 1H), 7.75 (s, 1H), 7.61 (d, J=7.4 Hz, 1H), 7.53 (t, J=7.6 Hz, 1H), 6.06 (s, 1H), 4.82 (dd, J=3.3 Hz, J=11.5 Hz, 1H), 4.31 (dd, J=3.9 Hz, J=13.6 Hz, 1H), 3.32-3.00 (m, 5H), 2.63 (d, J=12.0 Hz, 1H), 2.09 (d, J=13.3 Hz, 1H), 1.97 (td, J=5.0 Hz, J=12.9 Hz, 1H), 1.60 (t, J=12.3 Hz, 1H); ¹³C NMR (DMSO-d6; 101 MHz) δ 177.2 (Cq), 164.5 (Cq), 149.2 (Cq), 139.9 (Cq), 134.2 (Cq), 132.8 (Cq), 130.3 (CH), 129.8 (CH), 128.8 (CH), 121.0 (CH), 94.4 (CH), 65.4 (Cq), 57.1 (CH), 44.4 (CH₂), 39.6 (CH₂), 38.3 (CH₂), 38.0 (CH₂), 37.5 (CH₂); MS (IS) m/z: 418.0 [M+H]⁺.

### 1-(3-bromopyridin-2-yl)-3-(6-oxo-1,2,3,4,6,10b-hexahydropyrido[2,1-a]isoindol-10-yl)thiourea (72)

The compound 72 is obtained as a yellow solid according to general procedure B with a yield of 70% after purification on silica gel (CH₂Cl₂/MeOH: 99/1). MP: 180° C.; IR (ATR-Ge v cm⁻¹): 3403 (N—H amine), 1686 (C═O amide), 1566-1503-1417 (C═C arom), 1342 (C—N), 1286 (C—C), 757 (C—H arom); ¹H NMR (CDCl₃; 400 MHz) δ 13.25 (s, 1H), 8.84 (s, 1H), 8.20 (dd, J=1.5 Hz, 4.9 Hz, 1H), 7.99 (dd, J=1.6 Hz, 7.9 Hz, 1H), 7.83 (dd, J=7.5 Hz, 13.9 Hz, 2H), 7.52 (t, J=7.7 Hz, 1H), 6.99 (dd, J=4.9 Hz, 7.9 Hz, 1H), 4.59 (dd, J=3.7 Hz, 11.8 Hz, 1H), 4.49 (dd, J=4.1 Hz, 13.1 Hz, 1H), 2.98 (td, J=4.1 Hz, 13.1 Hz, 1H), 2.42 (dd, J=3.3 Hz, 12.9 Hz, 1H), 1.93 (d, J=13.5 Hz, 1H), 1.80 (d, J=13.2 Hz, 1H), 1.68-1.56 (m, 1H), 1.48-1.31 (m, 1H), 1.14 (qd, J=3.3 Hz, 12.9 Hz, 1H); ¹³C NMR (CDCl₃; 101 MHz) δ 179.4 (Cq), 165.6 (Cq), 149.7 (Cq), 144.6 (CH), 142.5 (CH), 140.8 (Cq), 134.1 (Cq), 133.4 (Cq), 129.3 (CH), 129.0 (CH), 122.7 (CH), 119.5 (CH), 107.0 (Cq), 58.7 (CH), 39.8 (CH₂), 30.5 (CH₂), 25.3 (CH₂), 23.8 (CH₂); MS (IS) m/z: 418.5 [M+H]⁺

### 1-(5-methylpyridin-2-yl)-3-(6-oxo-1,2,3,4,6,10b-hexahydropyrido[2,1-a]isoindol-10-yl)thiourea (73)

The compound 73 is obtained according to the procedure B from isothiocyanate 11 as a white solid with 60% of yield after flash chromatography on silica gel (CH₂Cl₂/MeOH: 99/1). MP: 232° C.; IR (ATR-Ge v cm⁻¹): 3297 (N—H amine), 1673 (C═O amide), 1540-1506-1488 (C═C arom), 1170 (C—N); ¹H NMR (DMSO-d6; 400 MHz) δ 13.74 (s, 1H), 11.02 (s, 1H), 8.16 (s, 1H), 8.10 (d, J=7.6 Hz, 1H), 7.71 (dd, J=2.1 Hz, 8.5 Hz, 1H), 7.58 (d, J=6.9 Hz, 1H), 7.52 (t, J=7.6 Hz, 1H), 7.20 (d, J=8.5 Hz, 1H), 4.65 (dd, J=3.5 Hz, 11.6 Hz, 1H), 4.24 (dd, J=3.5 Hz, 12.7 Hz, 1H), 3.02 (td, J=3.5 Hz, 12.8 Hz, 1H), 2.44-2.32 (m, 1H), 2.27 (s, 3H), 1.83 (d, J=12.9 Hz, 1H), 1.73 (d, J=12.7 Hz, 1H), 1.67-1.49 (m, 1H), 1.31-1.14 (m, 1H), 1.01-0.96 (m, 1H); ¹³C NMR (DMSO-d6; 101 MHz) δ 178.6 (Cq), 164.3 (Cq), 151.4 (Cq), 144.6 (CH), 140.3 (CH), 139.9 (Cq), 134.1 (Cq), 133.0 (Cq), 128.5 (CH), 128.4 (CH), 127.6 (Cq), 120.5 (CH), 112.5 (CH), 57.6 (CH), 38.9 (CH₂), 30.0 (CH₂), 24.9 (CH₂), 22.9 (CH₂), 17.2 (CH₃). MS (IS) m/z: 353.0 [M+H]⁺

### 1-(3-Bromopyridin-2-yl)-3-(6′-oxo-3′,4′,6′,10b′-tetrahydro-1′H-spiro[[1,3]dithiolane-2,2′-pyrido[2,1-a]isoindole]-10′-yl)thiourea (74)

The compound 74 was obtained according to procedure B with a yield of 70% as a white solid. m.p. 215° C. IR (ATR-Ge v cm⁻¹): 3380 (N—H amine), 1686 (C═O amide), 1554-1503 (C═C arom), 1336 (C—N), 1287 (C—C), 754 (C—H arom). ¹H NMR (CDCl₃, 400 MHz): 13.4 (s, 1H), 8.83 (s, 1H), 8.35 (dd, J=2.5 Hz, J=7.5 Hz, 1H), 8.16 (d, J=7.5 Hz, 1H), 7.98 (dd, J=2.5 Hz, J=7.5 Hz, 1H), 7.77 (d, J=7.5 Hz, 1H), 7.53 (t, J=7.5 Hz, 1H), 6.97 (dd, J=5.0 Hz, J=7.5 Hz, 1H), 4.85 (dd, J=5.0 Hz, J=12.5 Hz, 1H), 4.53 (dd, J=2.5 Hz, J=12.5 Hz, 1H), 3.05-3.34 (m, 5H), 2.86 (d, J=12.5 Hz, 1H), 2.20 (d, J=12.5 Hz, 1H), 2.01 (td, J=5.0 Hz, J=12.5 Hz, 1H), 1.77 (t, J=12.5 Hz, 1H). ¹³C NMR (CDCl₃, 100 MHz): 178.9 (Cq), 165.5 (Cq), 149.6 (Cq), 144.8 (CH), 142.4 (CH), 138.2 (Cq), 133.4 (Cq), 133.3 (Cq), 128.9 (CH), 128.8 (CH), 122.2 (CH), 119.1 (CH), 106.8 (Cq), 65.5 (Cq), 57.6 (CH), 45.4 (CH₂), 41.0 (CH₂), 39.0 (CH₂), 38.6 (CH₂), 38.2 (CH₂). HRMS: calc. for C₂₀H₂₀N₄OS₃Br: 506.9983; found 506.9998.

2.3. General Procedure C

Synthesis of the Derivatives of Ureas (75) and (76) from Thioureas (72) or (73)

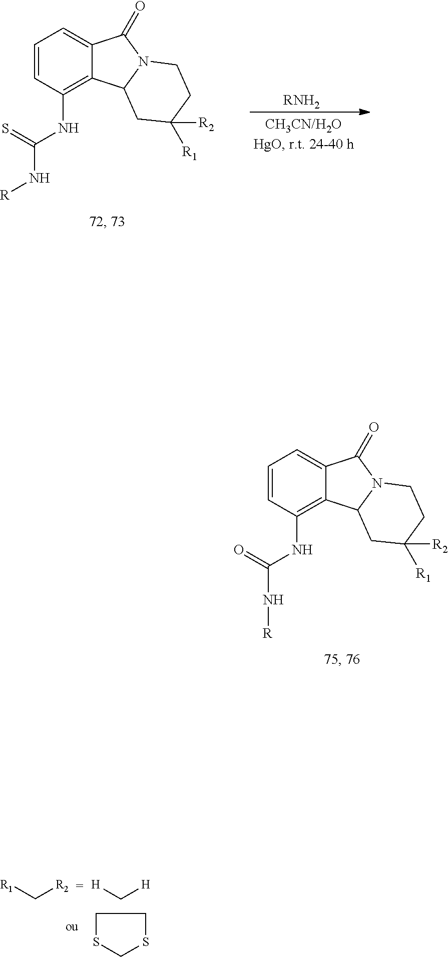

To a solution of thiourea (0.3 mmol) in a water/acetonitrile mixture 1/1 (20 mL), were added 390 mg of mercury oxide (II) (1.8 mmol, 6.0 equ.). The reaction mixture is stirred at room temperature in darkness for 24 hours to 40 hours and then filtered on celite. The filtrate is evaporated under reduced pressure and purified by chromatography on silica gel, in order to obtain the expected ureas.

### 1-(3-Bromopyridin-2-yl)-3-(6-oxo-1,2,3,4,6,10b-hexahydropyrido[2,1-a]isoindol-10-yl)urea (75)

The compound 75 is obtained as a pale yellow solid according to the general procedure C from 72 with a yield of 62% after purification on silica gel (CH₂Cl₂/MeOH: 99/1). MP: 220° C.; IR (ATR-Ge v cm⁻¹): 1678 (C═O amide), 1567 (N—H amide), 1478-1432-1391 (C═C arom), 1283 (C—N), 1203-1025 (C—C), 739 (C—Br); ¹H NMR (CDCl₃, 400 MHz) δ 11.60 (s, 1H), 8.15-8.05 (m, 2H), 7.87 (dd, J=1.5 Hz, J=7.9 Hz, 1H), 7.60-7.49 (m, 2H), 7.39 (t, J=7.8 Hz, 1H), 6.87 (dd, J=4.9 Hz, J=7.9 Hz, 1H), 4.46 (dd, J=4.9 Hz, J=13.3 Hz, 1H), 4.39 (dd, J=3.5 Hz, J=11.7 Hz, 1H), 2.94 (td, J=3.5 Hz, J=13.0 Hz, 1H), 2.64 (dd, J=3.1 Hz, J=13.0 Hz, 1H), 1.96 (d, J=13.3 Hz, 1H), 1.76 (d, J=13.2 Hz, 1H), 1.69-1.55 (m, 1H), 1.43-1.29 (m, 1H), 1.02 (qd, J=3.1 Hz, J=13.0 Hz, 1H); ¹³C NMR (CDCl₃, 101 MHz) δ 166.0 (Cq), 151.5 (Cq), 149.6 (Cq), 144.5 (CH), 142.3 (CH), 135.9 (Cq), 133.6 (Cq), 132.9 (Cq), 129.3 (CH), 124.2 (CH), 119.7 (CH), 118.7 (CH), 107.4 (Cq), 58.5 (CH), 39.9 (CH₂), 30.4 (CH₂), 25.4 (CH₂), 24.1 (CH₂); MS (IS) m/z: 402.5 [M+H]⁺.

### 1-(5-methylpyridin-2-yl)-3-(6-oxo-1,2,3,4,6,10b-hexahydropyrido[2,1-a]isoindol-10-yl)urea (76)

The compound 76 is obtained according to procedure C from the derivative 73 as a white solid after flash chromatography on silica gel (CH₂Cl₂/MeOH: 99/1). IR (ATR-Ge v cm⁻¹): ¹H NMR (CDCl₃; 400 MHz) δ 11.86 (s, 1H), 8.22 (d, J=8.1 Hz, 1H), 8.03 (s, 1H), 7.63 (d, J=7.4 Hz, 1H), 7.60-7.39 (m, 3H), 6.73 (s, 1H), 4.55 (dd, J=4.6 Hz, 13.2 Hz, 1H), 4.49 (dd, J=3.4 Hz, 12.3 Hz, 1H), 3.03 (td, J=3.5 Hz, 12.3 Hz, 1H), 2.81 (d, J=10.0 Hz, 1H), 2.33 (s, 3H), 2.05 (d, J=13.8 Hz, 1H), 1.84 (d, J=14.6 Hz, 1H), 1.78-1.68 (m, 1H), 1.50-1.41 (m, 1H), 1.16-1.02 (m, 1H); MS (IS) m/z: 337.5 [M+H]⁺, 673.5 [2M+H]⁺

2.4. General Procedure D

Synthesis of the Ureas (77) to (96) from the Amines (5), (7) or (10).

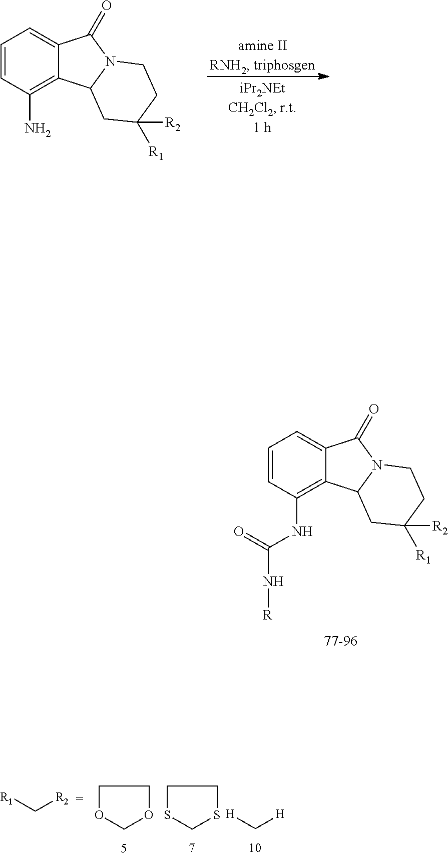

To a solution of triphosgen (29 mg, 0.36 equ.) in dichloromethane (2 mL) is slowly added for one hour by means of a syringed pump a solution of the amine I (0.27 mmol) and 84 μL of i-Pr₂NEt (0.59 mmol; 2.2 equ.) in dichloromethane (3 mL). The reaction mixture is stirred for 10 min, and then a solution of the amine II (0.32 mmol, 1.2 equ.) and of i-Pr₂NEt (84 μL, 0.59 mmol; 2.2 equ.) in dichloromethane (3 mL) is added. The reaction mixture is stirred for 1 h and then dichloromethane (4 mL) and an aqueous solution of saturated NaHCO₃ (5 mL) are added. After decantation, the organic phases are washed with an aqueous solution saturated with NaCl (5 mL). The organic phase is dried on MgSO₄ filtered and then evaporated under reduced pressure in order to obtain the expected urea which will be purified by flash chromatography on silica gel.

### 1-(6′-Oxo-3′,4′,6′,10b′-tetrahydro-1′H-spiro[[1,3]dithiolane-2,2′-pyrido[2,1-a]isoindole]-10′-yl)-3-(1H-pyrazol-3-yl)urea (77)

The compound 77 is obtained as a pale beige solid from the amine 7 according to the general procedure D after chromatography on silica gel (dichloromethane/methanol 98/2). MP: 184° C.; IR (ATR-Ge v cm⁻¹): 1731-1691 (C═O amide), 1620-1606 (N—H amide), 1575-1528-1488 (C═C arom), 1386-1242 (C—N), 1042 (C—C); ¹H NMR (DMSO-d6; 400 MHz) δ 9.88 (s, 1H), 8.05 (d, J=2.8 Hz, 1H), 7.66 (dd, J=1.0 Hz, 7.6 Hz, 1H), 7.59-7.48 (m, 2H), 5.90 (d, J=2.8 Hz, 1H), 5.40 (s, 2H), 4.89 (dd, J=3.3 Hz, J=11.5 Hz, 1H), 4.30 (dd, J=3.5 Hz, J=13.7 Hz, 1H), 3.46-3.22 (m, 4H), 3.12 (td, J=3.5 Hz, J=13.2 Hz, 1H), 2.59 (d, J=11.9 Hz, 1H), 2.11 (d, J=13.4 Hz, 1H), 1.96 (td, J=5.1 Hz, J=12.9 Hz, 1H), 1.55 (t, 12.0 Hz, 1H); ¹³C NMR (DMSO-d6; 101 MHz) δ 164.4 (Cq), 158.0 (Cq), 147.7 (Cq), 138.1 (Cq), 132.8 (Cq), 132.3 (Cq), 129.9 (CH), 128.8 (CH), 127.5 (CH), 120.0 (CH), 99.6 (CH), 65.5 (Cq), 57.2 (CH), 43.9 (CH₂), 39.7 (CH₂), 38.2 (CH₂), 38.0 (CH₂), 37.6 (CH₂); MS (IS) m/z: 402.0 [M+H]⁺

### 3-Amino-N-(6-oxo-1,2,3,4,6,10b-hexahydropyrido[2,1-a]isoindol-10-yl)-1H-pyrazole-1-carboxamide (78)

The compound 78 is obtained with a yield of 63% as a pale beige solid from the amine 10 according to the general procedure D after chromatography on silica gel (dichloromethane/methanol 98/2). MP: 166° C.; IR (ATR-Ge v cm⁻¹): 3320 (NH amine), 1685 (C═O amide), 1572 (N—H amide), 1520-1482-1430 (C═C arom), 1384 (C—N), 1286-1240-1046 (C—C), 758 (C—H arom); ¹H NMR (CDCl₃; 400 MHz) δ 8.77 (s, 1H), 8.05 (d, J=2.8 Hz, 1H), 7.95 (d, J=8.0 Hz, 1H), 7.69 (d, J=7.5 Hz, 1H), 7.48 (t, J=7.8 Hz, 1H), 5.91 (d, J=2.9 Hz, 1H), 4.61-4.42 (m, 2H), 4.12 (s, 2H), 2.99 (td, J=3.4 Hz, J=13.0 Hz, 1H), 2.49 (dd, J=3.3 Hz, J=12.9 Hz, 1H), 1.99 (d, J=13.6 Hz, 1H), 1.82 (d, J=13.2 Hz, 1H), 1.70 (qt, J=3.3 Hz, J=13.3 Hz, 1H), 1.49-1.32 (m, 1H), 1.12 (qd, J=3.3 Hz, J=12.9 Hz, 1H); ¹³C NMR (CDCl₃; 101 MHz) δ 165.7 (Cq), 156.7 (Cq), 147.2 (Cq), 136.5 (Cq), 134.0 (Cq), 131.80 (Cq), 130.4 (CH), 129.4 (CH), 124.3 (CH), 120.5 (CH), 99.7 (CH), 58.2 (CH), 39.9 (CH₂), 30.4 (CH₂), 25.3 (CH₂), 23.7 (CH₂); MS (IS) m/z: 312.5 [M+H]⁺, 623.5 [2M+H]⁺.

### N,N-Diisopropyl-W-(6-oxo-1,2,3,4,6,10b-hexahydropyrido[2,1-a]iso-indol-10-yl)urea (79)

The compound 79 is obtained with a yield of 70% as a white solid from the amine 10 according to the general procedure D after chromatography on silica gel (CH₂Cl₂/MeOH: 9/1+1% Et₃N). MP: 156° C.; IR (NaCl v cm⁻¹): 3425 (N—H amide), 1673 (C═O amide), 1518 (N—H amide), 1435 (C═C arom), 1289-1248 (C—C); ¹H NMR (CDCl₃; 400 MHz) δ 7.60 (dd, J=6.0 Hz, J=2.4 Hz, 1H), 7.40-7.32 (m, 2H), 6.30 (sb, 1H), 4.55 (dd, J=11.7 Hz, J=3.6 Hz, 1H), 4.46 (dd, J=13.3 Hz, J=4.9 Hz, 1H), 3.96 (quint, J=6.8 Hz, 1H), 2.97 (td, J=12.9 Hz, J=3.5 Hz, 1H), 2.30-2.24 (m, 1H), 1.97-1.92 (m, 1H), 1.82-1.75 (m, 1H), 1.61 (qt, J=13.1 Hz, J=3.2 Hz, 1H), 1.35 (m, 12H), 1.41-1.32 (m, 1H), 1.00 (dd, J=11.8 Hz, J=3.5 Hz, 1H); ¹³C NMR (CDCl₃, 101 MHz) (δ 166.0 (Cq), 153.9 (Cq), 137.7 (Cq), 134.2 (2×Cq), 128.7 (CH), 125.3 (CH), 119.6 (CH), 59.2 (CH), 46.2 (CH), 39.7 (CH₂), 29.9 (CH₂), 25.4 (CH₂), 23.7 (CH₂), 21.8 (2×CH₃), 21.3 (2×CH₃). MS (IS) m/z: 393.5 [M+H]⁺

### N-[2-(Dimethylamino)ethyl]-N′-(6′-oxo-3′,4,4′,5,6′,1′H-hexahydro-1′H-spiro[1,3-dithiolane-2,2′-pyrido[2,1-a]isoindol]-10′-yl)urea (80)

The compound 80 is obtained with a yield of 50% as a white solid from the amine 7 according to the general procedure D after chromatography on silica gel (CH₂Cl₂/MeOH: 9/1+1% Et₃N). MP: 194° C.; IR (NaCl v cm⁻¹): 3384 (N—H amide), 1654 (C═O amide), 1486-1426 (C═C arom), 1291 (C—C); ¹H NMR (CDCl₃; 400 MHz) (δ 8.20-7.80 (sb, 1H), 7.61 (d, J=7.5 Hz, 1H), 7.50 (d, J=6.6 Hz, 1H), 7.38 (t, J=7.7 Hz, 1H), 5.89 (sb, 1H), 4.74 (dd, J=11.5 Hz, J=3.2=Hz, 1H), 4.47 (dd, J=13.7 Hz, J=5.0 Hz, 1H), 3.43-3.32 (m, 6H), 3.20 (td, J=13.2 Hz, J=3.2 Hz, 1H), 2.82 (d, J=11.6 Hz, 2H), 2.55-2.46 (m, 2H), 2.26 (s, 6H), 2.15 (d, J=12.0 Hz, 1H), 1.97 (td, J=12.9 Hz, J=5.0 Hz, 1H), 1.59 (dd, J=12.9 Hz, J=11.9 Hz, 1H); ¹³C NMR (CDCl₃, 101 MHz) (δ 166.1 (Cq), 156.2 (Cq), 136.4 (Cq), 134.1 (Cq), 133.5 (Cq), 129.4 (CH), 126.0 (CH), 119.7 (CH), 65.7 (Cq), 59.4 (CH₂), 58.2 (CH), 45.3 (2×CH₃), 45.0 (CH₂), 41.4 (CH₂), 39.3 (CH₂), 38.7 (2×CH₂), 38.4 (CH₂); MS (m/z) (IS): 407.0 [M+H]⁺.

### N-Cyclohexyl-N′-(6′-oxo-3′,4,4′,5,6′,10b′-hexahydro-1′H-spiro[1,3-dithiolane-2,7-pyrido[2,1-a]isoindol]-10′-yl)urea (81)

The compound 81 is obtained with a yield of 64% as a white solid from the amine 7 according to the general procedure D after chromatography on silica gel (CH₂Cl₂/MeOH 98/2+1% Et₃N). MP: 172° C.; IR (NaCl v cm⁻¹): 3349 (N—H amide), 1652 (C═O amide), 1559 (C═C arom), 1510 (N—H amide), 1486-1452-1425 (C═C arom), 1287-1254 (C—C); ¹H NMR (CDCl₃; 400 MHz) (δ 7.78 (sb, 1H), 7.44 (d, J=7.4 Hz, 1H), 7.39 (d, J=7.4 Hz, 1H), 7.28 (t, J=7.7 Hz, 1H), 5.80 (d, J=7.5 Hz, 1H), 4.70 (dd, J=11.5 Hz, J=3.2 Hz, 1H), 4.39 (dd, J=13.5 Hz, J=3.9 Hz, 1H), 3.62-3.61 (m, 1H), 3.29-3.14 (m, 5H), 2.83 (d, J=11.9 Hz, 1H), 2.11-2.09 (m, 2H), 2.00 (d, J=9.5 Hz, 1H), 1.95-1.89 (m, 2H), 1.67 (sb, 2H), 1.59-1.56 (m, 1H), 1.45 (t, J=12.3 Hz, 1H), 1.34-1.26 (m, 2H), 1.10-1.08 (m, 2H); ¹³C NMR (CDCl₃; 101 MHz) (δ 166.3 (Cq), 155.2 Cq), 136.5 (Cq), 134.2 (Cq), 133.3 (Cq), 129.2 (CH), 126.0 (CH), 119.1 (CH), 65.5 (Cq), 58.4 (CH), 49.0 (CH), 44.9 (CH₂), 41.3 (CH₂), 39.3 (CH₂), 38.9 (CH₂), 38.4 (CH₂), 33.9 (CH₂), 33.6 (CH₂), 25.7 (CH₂), 25.0 (CH₂), 25.0 (CH₂); MS (IS) m/z: 418.5 [M+H]⁺

### N-(6′-Oxo-3′,4,4′,5,6′,10b′-hexahydro-1′H-spiro[1,3-dithiolane-2,2′-pyrido[2,1-a]isoindol]-10′-yl)-N′-piperidin-1-yl-urea (82)

The compound 82 is obtained with a yield of 56% as a white solid from the amine 7 according to the general procedure D after chromatography on silica gel (CH₂Cl₂/MeOH 97/3+1% Et₃N). MP: 158° C.; IR (NaCl v cm⁻¹): 3444 (N—H amide), 1688 (C═O amide), 1521 (N—H amide), 1434 (C═C arom), 1289-1242 (C—C); ¹H NMR (CDCl₃; 400 MHz) δ 8.32 (sb, 1H), 8.23 (dd, J=8.0 Hz, J=0.9 Hz, 1H), 7.55 (dd, J=7.5 Hz, J=1.0 Hz, 1H), 7.44 (t, J=7.8 Hz, 1H), 6.03 (sb, 1H), 4.59 (dd, J=11.7 Hz, J=3.3 Hz, 1H), 4.53 (ddd, J=8.9 Hz, J=4.9 Hz, J=1.7 Hz, 1H), 3.46-3.25 (m, 5H), 3.25-3.17 (m, 2H), 2.84 (ddd, J=12.9 Hz, J=3.3 Hz, J=2.0 Hz, 1H), 2.45 (sb, 1H), 2.23-2.16 (m, 1H), 2.04 (sb, 1H), 2.00 (td, J=13.4 Hz, J=5.0 Hz, 1H), 1.85-1.68 (m, 6H), 1.26-1.19 (m, 1H); ¹³C NMR (CDCl₃, 101 MHz) δ 166.1 (Cq), 154.8 (Cq), 133.7 (Cq), 132.9 (Cq), 132.7 (Cq), 129.7 (CH), 122.8 (CH), 118.6 (CH), 65.6 (Cq), 57.9 (CH₂), 57.4 (CH), 45.6 (CH₂), 42.1 (CH₂), 39.8 (CH₂), 38.3 (CH₂), 38.3 (CH₂), 26.0 (CH₂), 25.9 (CH₂), 23.2 (CH₂); MS (IS) m/z: 419.5 [M+H]⁺.

### 4-Methyl-N-(6′-oxo-3′,4′,6′,10b′-tetrahydro-1′H-spiro[1,4-d]thiane-2,2′-pyrido[2,1-a]isoindol]-10′-yl)piperazine-1-carboxamide (83)

The compound 83 is obtained with a yield 66% as a white solid from the amine 7 according to the general procedure D after chromatography on silica gel (CH₂Cl₂/MeOH 97/3+1% Et₃N). MP: 168° C.; IR (NaCl v cm⁻¹): 3422 (N—H amide), 1643 (C═O amide), 1520 (C═C arom), 1429 (C═C arom), 1292 (C—C); ¹H NMR (CDCl₃, 250 MHz) δ 7.55 (dd, J=7.1 Hz, J=1.2=Hz, 1H), 7.34 (t, J=7.8 Hz, 1H), 7.27 (dd, J=7.8 Hz, J=1.2 Hz, 1H), 7.04 (sb, 1H), 4.75 (dd, J=11.6 Hz, J=3.2 Hz, 1H), 4.42 (dd, J=13.8 Hz, J=3.5 Hz, 1H), 3.72-3.44 (m, 5H), 3.41-3.34 (m, 4H), 3.21 (td, J=13.2 Hz, J=3.2 Hz, 1H), 2.68-2.61 (m, 1H), 2.50-2.42 (m, 1H), 2.33 (s, 1H), 2.18-2.07 (m, 2H), 1.94 (td, J=12.9 Hz, J=5.0 Hz, 1H), 1.56 (t, J=12.2 Hz, 1H); ¹³C NMR (CDCl₃, 101 MHz) δ 166.1 (Cq), 154.7 Cq), 137.6 (Cq), 134.2 (Cq), 133.6 (Cq), 129.2 (CH), 126.3 (CH), 120.2 (CH), 65.7 (Cq), 58.6 (CH), 55.1 (2×CH₂), 46.4 (CH₃), 45.1 (CH₂), 44.5 (2×CH₂), 41.6 (CH₂), 39.5 (CH₂), 38.6 (2×CH₂MS (IS) m/z: 419.5 [M+H]⁺, 451.5 [M+K]⁺.

### N-1-Adamantyl-N′-(6′-oxo-3′,4′,5,6,6′,10b′-hexahydro-1H-spiro[1,4-d]thiane-2,7-pyrido[2,1-a]isoindol]-10′-yl)urea (84)

The compound 84 is obtained with a yield 81% as a white solid from the amine 7 according to the general procedure D after chromatography on silica gel (CH₂Cl₂/MeOH: 96/4). MP: 132° C.; IR (ATR-Ge v cm⁻¹): 1655 (C═O amide), 1545 (NH amide), 1219 (C—C); ¹H NMR (CDCl₃; 400 MHz) (δ 7.63 (d, J=7.2 Hz, 1H), 7.46-7.40 (m, 2H), 6.32 (sb, 1H), 4.79 (dd, J=11.6 Hz, J=3.4 Hz, 1H), 4.63 (sb, 1H), 4.50 (dd, J=11.7 Hz, J=4.8 Hz, 1H), 3.38-3.35 (m, 4H), 3.25 (td, J=13.3 Hz, J=3.2 Hz, 1H), 2.83-2.81 (m, 1H), 2.19 (d, J=12.7 Hz, 1H), 2.10 (m, 3H), 2.05-1.97 (m, 8H), 1.69 (s, 6H); ¹³C NMR (CDCl₃ 101 MHz) (δ 166.4 Cq), 154.2 Cq), 136.4 (Cq), 134.3 (Cq), 133.4 (Cq), 129.4 (CH), 125.9 (CH), 119.2 (CH), 65.5 (Cq), 58.4 (CH), 51.4 Cq), 45.0 (CH₂), 42.4 (3×CH₂), 41.4 (CH₂), 39.4 (SCH₂), 39.0 (CH₂), 38.5 (CH₂), 36.5 (3×CH₂), 29.7 (3×CH); MS (IS) m/z: 470.5 [M+H]⁺, 492.5 [M+Na]⁺.

### N-(6-Oxo-1,2,3,4,6,10b-hexahydropyrido[2,1-a]isoindol-10-yl)-N′-(piperidin-1-yl)urea (85)

The compound 85 is obtained with a yield of 60% as a white solid from the amine 10 according to the general procedure D after chromatography on silica gel (CH₂Cl₂/MeOH: 96/4). MP: 166° C.; IR (ATR-Ge v cm⁻¹): 3306 (NH), 1692-1627 (C═O amide), 1557 (NH amide), 1441 (C—C); ¹H NMR (CDCl₃; 400 MHz) (δ 8.03 (sb, 1H), 7.95 (d, J=7.3 Hz, 1H), 7.35 (t, J=7.6 Hz, 1H), 7.27 (d, J=7.1 Hz, 1H), 6.55 (d, J=7.6 Hz, 1H), 4.46 (dd, J=11.3 Hz, J=2.9 Hz, 1H), 4.24 (dd, J=13.0 Hz, J=4.2 Hz, 1H), 3.32 (s, 1H), 2.99 (td, J=12.7 Hz, J=3.0 Hz, 1H), 1.91-1.80 (m, 3H), 1.74 (d, J=12.6 Hz, 1H), 1.68-1.53 (m, 4H), 1.35-1.14 (m, 6H), 0.78 (q, J=12.2 Hz, 1H); ¹H NMR (CDCl₃; 101 MHz) (δ 164.7 (Cq), 154.1 (Cq), 135.2 (Cq), 134.3 (Cq), 132.7 (Cq), 128.5 (CH), 122.0 (CH), 116.2 (CH), 39.0 (CH₂), 57.5 (CH), 47.8 (CH₂), 33.0 (CH₂), 32.8 (CH₂), 29.8 (CH₂), 25.2 (CH₂), 25.0 (CH₂), 24.2 (CH₂), 23.0 (CH₂); MS (IS) m/z: 328.5 [M+H]⁺.

### N-(6′-Oxo-3′,4′,5,6,6′,10b′-hexahydro-1′H-spiro[1,4-d]thiane-2,7-pyrido[2,1-a]isoindol]-10′-yl)-N′-phenylurea (86)

The compound 86 is obtained with a yield of 76% as a white solid from the amine 7 according to the general procedure D after chromatography on silica gel (CH₂Cl₂/MeOH 97/3+1% Et₃N). MP: 161° C.; IR (ATR-Ge v cm⁻¹): 1659-1594 (C═O amide), 1541 (NH amide), 1488-1447 (C═C arom), 1296 (C—C); ¹H NMR (CDCl₃; 400 MHz) (δ 8.44 (sb, 1H), 8.35 (sb, 1H), 7.54 (d, J=7.8 Hz, 1H), 7.45 (d, J=7.4 Hz, 1H), 7.33 (d, J=7.8 Hz, 2H), 7.27 (t, J=7.6 Hz, 1H), 7.19 (t, J=7.8 Hz, 2H), 6.98 (t, J=7.3 Hz, 1H), 4.64 (dd, J=11.4 Hz, J=3.1 Hz, 1H), 4.38 (dd, J=13.6 Hz, J=3.6 Hz, 1H), 3.21-3.03 (m, 5H), 2.79 (d, J=12.0 Hz, 1H), 2.09 (d, J=13.0 Hz, 1H), 1.92 (td, J=13.1 Hz, J=5.0 Hz, 1H), 1.44 (d, J=12.4 Hz, 1H) ¹³C NMR (CDCl₃; 101 MHz) (δ 166.2 (Cq), 153.7 Cq), 138.6 (Cq), 136.4 (Cq), 133.5 (Cq), 133.1 (Cq), 129.4 (CH), 129.1 (2×CH), 126.2 (CH), 123.5 (CH), 120.0 (2×CH), 119.4 (CH), 65.4 (Cq), 58.2 (CH), 44.9 (CH₂), 41.0 (CH₂), 39.2 (CH₂), 39.0 (CH₂), 38.4 (CH₂). MS (IS) m/z: 412.5 [M+H]⁺, 410.5 [M−H]⁺.*

### N-(6-Oxo-1,2,3,4,6,10b-hexahydropyrido[2,1-a]isoindol-10-yl)-N′-phenylurea (87)

The compound 87 is obtained with a yield of 22% as a white solid from the amine 10 according to the general procedure D after chromatography on silica gel (CH₂Cl₂/MeOH: 97/3+1% Et₃N). MP: 172° C.; IR (ATR-Ge v cm⁻¹): 1650 (CO amide), 1290 (C—C); ¹H NMR (CDCl₃; 400 MHz) (δ 8.41 (sb, 1H), 8.25 (sb, 1H), 7.62 (d, J=7.4 Hz, 1H), 7.50 (d, J=7.5 Hz, 1H), 7.34-7.26 (m, 2H), 7.26-7.18 (m, 2H), 6.99 (t, J=7.3 Hz, 1H), 4.50-4.37 (m, 2H), 2.89 (td, J=12.5 Hz, J=2.9 Hz, 1H), 2.56 (d, J=10.2 Hz, 1H), 1.80-1.72 (m, 2H), 1.30-1.23 (m, 1H), 1.46 (q, J=12.9 Hz, 1H), 0.92 (qd, J=12.0 Hz, J=3.0 Hz, 1H); ¹³C NMR (CDCl₃, 101 MHz) (δ 166.6 (Cq), 153.6 Cq), 138.7 (Cq), 137.5 (Cq), 133.9 (Cq), 133.1 (Cq), 129.2 (3×CH), 125.8 (CH), 123.4 (CH), 119.7 (CH), 119.2 (2×CH), 54 9 (CH), 40.1 (CH₂), 30.1 (CH₂), 25.5 (CH₂), 23.5 (CH₂); MS (IS) m/z: 322.5 [M+H]⁺.

### N-Benzyl-N′-(6′-oxo-3′,4,4′,5,6′,10b′-hexahydro-1′H-spiro[1,3-dithiolane-2,2′-pyrido[2,1-a]isoindol]-10′-yl)urea (88)

The compound 88 is obtained with a yield of 69% as a white solid from the amine 7 according to the general procedure D after chromatography on silica gel (CH₂Cl₂/MeOH 97/3). MP: 165° C.; IR (ATR-Ge v cm⁻¹): 3327 (NH amide), 1663 (C═O amide), 1541 (NH amide), 1500-1455-1427 (C═C arom), 1231 (C—C); ¹H NMR (CDCl₃; 400 MHz) (δ 7.81 (s, 1H), 7.54 (d, J=7.9 Hz, 1H), 7.32 (d, J=7.4 Hz, 1H), 7.28-7.22 (m, 6H), 6.25 (sb, 1H), 4.60 (dd, J=14.9 Hz, J=3.4 Hz, 1H), 4.42-4.33 (m, 2H), 4.30 (dd, J=13.6 Hz, J=3.9 Hz, 1H), 3.26-3.03 (m, 4H), 2.77 (d, J=12.0 Hz, 1H), 2.04 (d, J=13.1 Hz, 1H), 1.86 (td, J=13.0 Hz, J=5.0 Hz, 1H), 1.45 (t, J=12.4 Hz, 1H); ¹³C NMR (CDCl₃; 101 MHz) (δ 166.3 (Cq), 156.0 (Cq), 139.1 (Cq), 136.4 (Cq), 134.0 (Cq), 133.2 (Cq), 129.4 (CH), 128.8 (2×CH), 127.5 (2×CH), 127.5 (CH), 126.4 (CH), 119.4 (CH), 65.4 (Cq), 58.1 (CH), 44.9 (CH₂), 44.3 (CH₂), 41.2 (CH₂), 39.2 (SCH₂), 38.9 (SCH₂), 38.5 (CH₂); MS (IS) m/z: 426.5 [M+H]⁺, 424.0 [M−H]⁺.

### N-(2-Methoxyphenyl)-N′-(6′-oxo-3′,4,4′,5,6′,10b′-hexahydro-1′H-spiro[1,3-dithiolane-2,2′-pyrido[2,1-a]isoindol]-10′-yl)urea (89)

The compound 89 is obtained with a yield of 67% as a white solid from the amine 7 according to the general procedure D after chromatography on silica gel (CH₂Cl₂/MeOH: 97/3+1% Et₃N). MP: 178° C.; IR (NaCl v cm⁻¹): 3345 (N—H amide), 1659 (C═O amide), 1599 (C═C arom), 1540.4 (N—H amide), 1487-1459-1433 (C═C arom), 1287-1251 (C—C); ¹H NMR (CDCl₃; 400 MHz) (δ 8.48 (sb, 1H), 8.22 (dd, J=7.4 Hz, J=2.0 Hz, 1H), 8.16 (sb, 1H), 7.79 (d, J=7.9 Hz, 1H), 7.57 (d, J=7.6 Hz, 1H), 7.34 (t, J=7.8 Hz, 1H), 7.02-6.91 (m, 2H), 6.83 (dd, J=7.6 Hz, J=1.9 Hz, 1H), 4.77 (dd, J=11.5 Hz, J=3.2 Hz, 1H), 4.42 (dd, J=13.4 Hz, J=3.4 Hz, 1H), 3.65 (s, 3H), 3.25-2.86 (m, 6H), 2.09 (d, J=13.4 Hz, 1H), 2.01-1.95 (m, 1H), 1.55 (t, J=12.3 Hz, 1H); ¹³C NMR (CDCl₃; 101 MHz) (δ 166.6 Cq), 153.2 Cq), 148.5 (Cq), 135.8 (Cq), 134.0 (Cq), 133.2 (Cq), 129.4 (CH), 128.3 (Cq), 126.0 (CH), 123.0 (CH), 121.3 (CH), 120.0 (CH), 119.4 (CH), 110.3 (CH), 65.4 (Cq), 58.2 (CH), 55.8 (CH₃), 45.2 (CH₂), 41.1 (CH₂), 39.3 (CH₂), 39.2 (CH₂), 38.4 (CH₂); MS (IS) m/z: 442.5 [M+H]⁺, 464.5 [M+Na]⁺.

### N-(3-Methoxyphenyl)-N′-(6′-oxo-3′,4,4′,5,6′,10b′-hexahydro-1′H-spiro[1,3-dithiolane-2,2′-pyrido[2,1-a]isoindol]-10′-yl)urea (90)

The compound 90 is obtained with a yield of 60% as a white solid from the amine 7 according to the general procedure D after chromatography on silica gel (CH₂Cl₂/MeOH 98/2). MP: 182° C.; IR (NaCl v cm⁻¹): 3345 (N—H amide), 1652 (C═O amide), 1572 (N—H amide), 1470 (C═C arom), 1251 (C—C); ¹H NMR (CDCl₃; 400 MHz) (δ 8.27 (sb, 1H), 8.18 (sb, 1H), 7.67 (d, J=7.8 Hz, 1H), 7.47 (d, J=7.0 Hz, 1H), 7.31 (t, J=7.8 Hz, 1H), 7.12-7.09 (m, 1H), 7.09 (t, J=8.1 Hz, 1H), 6.79 (dd, J=7.9 Hz, J=1.0 Hz, 1H), 6.53 (dd, J=8.2 Hz, J=1.9 Hz, 1H), 4.59 (dd, J=11.4 Hz, J=2.8 Hz, 1H), 4.37 (dd, J=12.9 Hz, J=3.0 Hz, 1H), 3.70 (s, 3H), 3.21-2.76 (m, 5H), 2.78 (d, J=11.9 Hz, 1H), 2.09 (d, J=14.6 Hz, 1H), 1.96-1.92 (m, 1H), 1.51 (t, J=12.3 Hz, 1H); ¹³C NMR (CDCl₃; 101 MHz) (δ 166.4 (Cq), 160.4 (Cq), 153.5 (Cq), 139.8 (Cq), 135.9 (Cq), 133.6 (Cq), 133.1 (Cq), 129.8 (CH), 129.5 (CH), 126.0 (CH), 119.3 (CH), 112.2 (CH), 109.0 (CH), 106.0 (CH), 65.4 (Cq), 58.2 (CH), 55.4 (CH₃), 45.0 (CH₂), 41.1 (CH₂), 39.2 (CH₂), 39.0 (CH₂), 38.5 (CH₂); MS (IS) m/z: 352.0 [M+H]⁺, 381.0 [M+K]⁺.

### N-(4-Methoxyphenyl)-N′-(6′-oxo-3′,4,4′,5,6′,10b′-hexahydro-1′H-spiro[1,3-dithiolane-2,2′-pyrido[2,1-a]isoindol]-10′-yl)urea (91)

The compound 91 is obtained with a yield of 60% as a white solid from the amine 7 according to the general procedure D after chromatography on silica gel (CH₂Cl₂/MeOH 98/2). MP: 183° C.; IR (NaCl v cm⁻¹): 3344 (N—H amide), 1651 (C═O amide), 1557 (C═C arom), 1511 (N—H amide), 1427 (C═C arom), 1293-1243 (C—C); ¹H NMR (CDCl₃; 400 MHz) δ 7.94 (sb, 1H), 7.86 (sb, 1H), 7.51-7.44 (m, 2H), 7.25 (t, J=7.7 Hz, 1H), 7.12 (d, J=8.9 Hz, 2H), 6.70 (d, J=8.9 Hz, 2H), 4.49 (dd, J=11.4 Hz, J=3.0 Hz, 1H), 4.36 (dd, J=13.0 Hz, J=3.4 Hz, 1H), 3.68 (s, 3H), 3.25-3.12 (m, 4H), 3.06 (d, J=12.8 Hz, 1H), 2.67 (d, J=11.6 Hz, 1H), 2.07 (d, J=13.7 Hz, 1H), 1.96-1.84 (m, 1H), 1.88 (td, J=12.9 Hz, J=4.8 Hz, 1H), 1.41 (t, J=12.4 Hz, 1H); ¹³C NMR (CDCl₃; 101 MHz) δ 166.1 (Cq), 156.5 (Cq), 154.1 Cq), 136.3 (Cq), 133.5 (Cq), 133.2 (Cq), 130.9 (Cq), 129.3 (CH), 126.0 (CH), 123.2 (2×CH), 119.5 (CH), 114.4 (2×CH), 65.5 (Cq), 58.1 (CH), 55.5 (OCH₃), 44.8 (CH₂), 41.2 (CH₂), 39.3 (CH₂), 38.8 (CH₂), 38.5 (CH₂); MS (IS) m/z: 442.5 [M+H]⁺.

### N-Benzyl-N′-(6-oxo-1,2,3,4,6,10b-hexahydropyrido[2,1-a]isoindol-10-yl)urea (92)

The compound 92 is obtained with a quantative yield as a white solid from the amine 10 according to the general procedure D after chromatography on silica gel (CH₂Cl₂/MeOH 98/2). MP: 183° C.; IR (ATR Ge v cm⁻¹): 3310 (NH amide), 1696-1631 (C═O amide), 1570 (NH amide), 1525-1480-1419 (C═C arom), 1276 (C—C); ¹H NMR (DMSO-d6; 400 MHz) δ 8.35 (s, 1H), 8.32 (sb, 1H), 7.94 (d, J=7.9 Hz, 1H), 7.38-7.26 (m, 5H), 7.10 (t, J=7.1 Hz, 1H), 4.48 (dd, J=11.4 Hz, J=3.2 Hz, 1H), 4.32 (qd, J=15.0 Hz, J=5.5 Hz, 2H), 4.23 (qd, J=13.0 Hz, J=4.5 Hz, 1H), 2.98 (td, J=12.9 Hz, J=3.3 Hz, 1H), 2.54-2.50 (m, 1H), 1.86 (d, J=12.7 Hz, 1H), 1.74 (d, J=12.7 Hz, 1H), 1.57 (q, J=13.1 Hz, 1H), 1.23-1.21 (m, 1H), 0.77 (qd, J=12.3 Hz, J=2.6 Hz, 1H); ¹³C NMR (DMSO-d6; 101 MHz) δ 164.6 (Cq), 154.9 (Cq), 140.0 (Cq), 135.0 (Cq), 134.7 (Cq), 132.7 (Cq), 128.4 (CH), 128.2 (CH), 127.1 (2×CH), 126.7 (CH), 125.3 (CH), 122.3 (CH), 116.4 (CH), 57.5 (CH), 42.8 (—NCH₂), 38.9 (CH₂), 25.0 (CH₂), 23.0 (CH₂); MS (IS) m/z: 336.5 [M+H]⁺.

### N-(2-Methoxyphenyl)-M-(6-oxo-1,2,3,4,6,10b-hexahydropyrido[2,1-a]isoindol-10-yl)urea (93)

The compound 93 is obtained with a yield of 45% as a white solid from the amine 10 according to the general procedure D after chromatography on silica gel (CH₂Cl₂/MeOH 97/3+1% Et₃N). MP: 177° C.; IR (NaCl v cm⁻¹): 3344 (N—H amide), 1601 (C═O amide), 1542 (C═C arom), 1540.4 (N—H amide), 1489-1459-1430 (C═C arom), 1173-1121 (C—C); ¹H NMR (CDCl₃; 400 MHz) δ 8.53 (sb, 1H), 8.19 (dd, J=7.5 Hz, J=1.9 Hz, 1H), 8.14 (sb, 1H), 7.73 (d, J=7.9 Hz, 1H), 7.55 (d, J=6.8 Hz, 1H), 7.32 (t, J=7.7 Hz, 1H), 6.95 (qd, J=7.5 Hz, J=1.9 Hz, 2H), 6.80 (dd, J=7.8 Hz, J=2.0 Hz, 1H), 4.53 (dd, J=11.5 Hz, J=3.2 Hz, 1H), 4.42 (dd, J=13.0 Hz, J=3.5 Hz, 1H), 3.60 (s, 3H), 2.88 (td, J=13.0 Hz, J=3.2 Hz, 1H), 2.61 (dd, J=12.3 Hz, J=2.1 Hz, 1H), 1.82 (d, J=13.0 Hz, 1H), 1.71 (d, J=13.0 Hz, 1H), 1.47 (q, J=13.0 Hz, 1H), 1.28-1.21 (m, 1H), 0.92 (d, J=12.0 Hz, 1H); ¹³C NMR (CDCl₃; 101 MHz) (δ 166.5 (Cq), 153.4 (Cq), 148.4 (Cq), 137.3 (Cq), 133.9 (Cq), 133.2 (Cq), 129.1 (CH), 128.4 (Cq), 125.5 (CH), 122.9 (CH), 121.2 (CH), 119.7 (CH), 119.2 (CH), 110.3 (CH), 59.3 (CH), 55.6 (CH₃), 40.1 (CH₂), 30.2 (CH₂), 25.5 (CH₂), 23.5 (CH₂); MS (IS) m/z: 352.0 [M+H]⁺

### N-(3-Methoxyphenyl)-M-(6-oxo-1,2,3,4,6,10b-hexahydropyrido[2,1-a]isoindol-10-yl)urea (94)

The compound 94 is obtained with a yield of 58% as a white solid from the amine 10 according to the general procedure D after chromatography on silica gel (CH₂Cl₂/MeOH 98/2). MP: 183° C.; IR (NaCl v cm⁻¹): 3345 (N—H amide), 1652 (C═O amide), 1572 (N—H amide), 1470 (C═C arom), 1251 (C—C); ¹H NMR (CDCl₃; 400 MHz) (δ 8.15 (sb, 1H), 7.99 (sb, 1H), 7.67 (d, J=7.9 Hz, 1H), 7.55 (d, J=7.5 Hz, 1H), 7.35 (t, J=7.7 Hz, 1H), 7.16-7.13 (m, 2H), 6.82 (d, J=8.0 Hz, 1H), 6.59 (dd, J=8.3 Hz, J=2.0 Hz, 1H), 4.47 (dd, J=11.6 Hz, J=3.4 Hz, 1H), 4.43 (dd, J=13.2 Hz, J=4.3 Hz, 1H), 3.74 (s, 3H), 2.92 (td, J=13.0 Hz, J=3.2 Hz, 1H), 2.54 (d, J=15.0 Hz, 1H), 1.86 (d, J=13.4 Hz), 1H, 1.76 (d, J=12.7 Hz, 1H), 1.49 (q, J=13.2 Hz, 1H), 1.32-1.27 (m, 1H), 0.97 (qd, J=12.5 Hz, J=2.7 Hz), 1H; ¹³C NMR (CDCl₃; 101 MHz) (δ 166.6 Cq), 160.4 Cq), 153.5 (Cq), 139.9 (Cq), 137.5 (Cq), 133.7 (Cq), 133.2 (Cq), 129.9 (CH), 129.3 (CH), 125.8 (CH), 119.4 (CH), 112.2 (CH), 109.0 (CH), 105.9 (CH), 59.3 (CH), 55.4 (CH₃), 40.1 (CH₂), 30.2 (CH₂), 25.5 (CH₂), 23.6 (CH₂); MS (IS) m/z: 352.0 [M+H]⁺, 381.0 [M+K]⁺.

### N-(4-Methoxyphenyl)-M-(6-oxo-1,2,3,4,6,10b-hexahydropyrido[2,1-a]isoindol-10-yl)urea (95)

The compound 95 is obtained with a yield of 43% as a white solid from the amine 10 according to the general procedure D after chromatography on silica gel (CH₂Cl₂/MeOH 96/4+1% Et₃N). MP: 193° C.; IR (NaCl v cm⁻¹): 3417 (N—H amide), 1652 (C═O amide), 1553 (C═C arom), 1510 (N—H amide), 1428 (C═C arom), 1289-1242 (C—C). ¹H NMR (CDCl₃; 400 MHz) (δ 7.77 (sb, 1H), 7.71 (sb, 1H), 7.63 (d, J=7.9 Hz, 1H), 7.52 (d, J=7.4 Hz, 1H), 7.32 (t, J=7.7 Hz, 1H), 7.21 (d, J=8.8 Hz, 2H), 6.79 (d, J=8.8 Hz, 2H), 4.42-4.37 (m, 2H), 3.74 (s, 3H), 2.88 (td, J=13.0 Hz, J=3.1 Hz, 1H), 2.43 (d, J=2.4 Hz, 1H), 1.84 (d, J=13.4 Hz, 1H), 1.75-1.73 (m, 1H), 1.47 (q, J=13.1 Hz, 1H), 1.31-1.24 (m, 1H), 0.92 (qd, J=14.0 Hz, J=2.5 Hz, 1H); ¹³C NMR (CDCl₃; 101 MHz) (δ 166.4 (Cq), 158.8 (Cq), 154.0 Cq), 137.3 (Cq), 133.7 (Cq), 133.4 (Cq), 131.0 (Cq), 129.3 (2×CH), 125.6 (CH), 123.0 (CH), 119.4 (CH), 114.6 (2×CH), 59.1 (CH), 55.6 (CH₃), 40.0 (CH₂), 30.1 (CH₂), 25.4 (CH₂), 23.6 (CH₂); MS (IS) m/z: 352.0 [M+H]⁺.

### N-(6-Methoxypyridin-3-yl)-M-(6-oxo-1,2,3,4,6,10b-hexahydropyrido[2,1-a]isoindol-10-yl)urea (96)

The compound 96 is obtained with a yield of 72% as a white solid from the amine 10 according to the general procedure D after chromatography on silica gel (CH₂Cl₂/MeOH 97/3+1% Et₃N). MP: 175° C.; IR (ATR-Ge v cm⁻¹): 1655 (C═O amide), 1549 (NH amide), 1484-1427 (C═C arom), 1252 (C—C); ¹H NMR (CDCl₃; 400 MHz) (δ 8.62 (sb, 1H), 8.41 (sb, 1H), 8.08 (d, J=2.6 Hz, 1H), 7.77 (dd, J=2.7 Hz, J=8.8 Hz, 1H), 7.73 (d, J=7.8 Hz, 1H), 7.48 (d, J=7.8 Hz, 1H), 7.32 (t, J=7.8 Hz, 1H), 6.66 (d, J=8.8 Hz, 1H), 4.51 (dd, J=3.3 Hz, J=11.6 Hz, 1H), 4.39 (dd, J=4.4 Hz, J=13.0 Hz, 1H), 3.86 (s, 3H), 3.07 (q, J=7.3 Hz, 1H), 2.89 (td, J=3.2 Hz, J=13.0 Hz, 1H), 2.64 (m, 1H), 1.85 (d, J=12.7 Hz, 1H), 1.75 (d, J=13.3 Hz, 1H), 1.51 (q, J=13.3 Hz, 1H), 0.93 (qd, J=12.8 Hz, J=2.6 Hz, 1H); ¹³C NMR (CDCl₃; 101 MHz) (δ 166.6 (Cq), 160.7 (Cq), 153.9 (Cq), 138.7 (CH), 137.2 (Cq), 133.9 (Cq), 133.1 (Cq), 132.5 (CH), 129.4 (Cq), 129.3 (CH), 125.5 (CH), 119.1 (CH), 110.6 (CH), 59.4 (CH), 53.7 (OCH₃), 40.1 (CH₂), 30.2 (CH₂), 25.5 (CH₂), 23.5 (CH₂); MS (IS) m/z: 353.5 [M+H]⁺, m/z=351.0 [M−H]⁺.

2.5. General Procedure E

Synthesis of the Ketones (97) to (103) from the Corresponding Acetals.

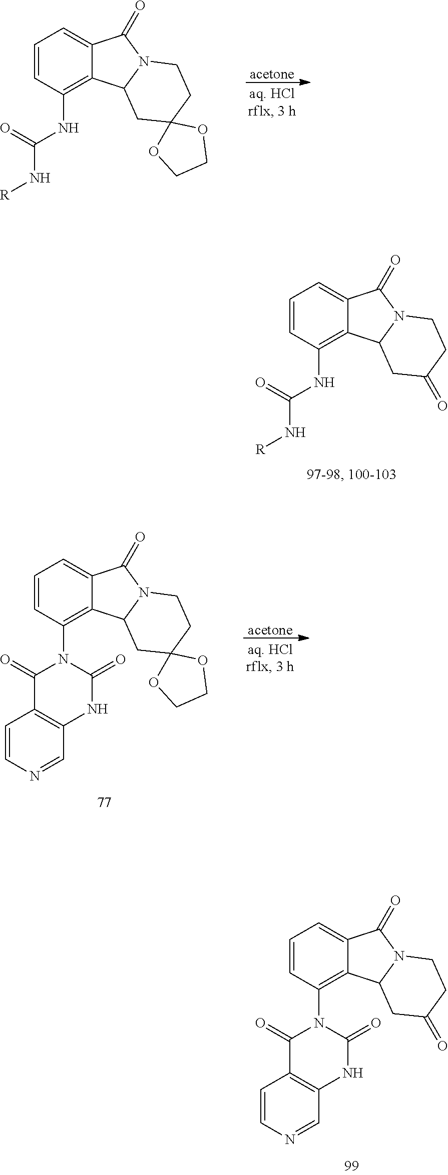

In a 50 mL flask, the acetal (0.52 mmol) is solubilized in 4 mL of acetone and an aqueous solution of 10% hydrochloric acid (2 mL) is then added. The reaction mixture is refluxed for three hours. After cooling, the reaction medium is concentrated to about 50% by evaporation and the precipitate is then filtered, washed with a little water (2 mL) and dried under reduced pressure in order to obtain the expected ketone product.

### 1-(2,6-Dioxo-1,2,3,4,6,10b-hexahydropyrido[2,1-a]isoindol-10-yl)-3-(pyridin-2-yl)urea (97)

The compound 97 is obtained according to the procedure E from the derivative 25 as a white solid with quantitative yield. MP: 254° C.; ¹H NMR (DMSO-d6; 400 MHz) δ 11.26 (s, 1H), 9.99 (s, 1H), 8.40-8.34 (m, 1H), 8.30 (d, J=8.0 Hz, 1H), 7.85-7.76 (m, 1H), 7.51 (t, J=7.7 Hz, 1H), 7.45 (d, J=6.8 Hz, 1H), 7.30 (d, J=8.3 Hz, 1H), 7.10 (dd, J=5.4 Hz, J=6.7 Hz, 1H), 5.08 (dd, J=3.9 Hz, 11.9 Hz, 1H), 4.48 (m, 1H), 3.49 (td, J=4.4 Hz, J=12.6 Hz, 1H), 3.26 (dd, J=2.8 Hz, J=14.0 Hz, 1H), 2.66-2.57 (m, 1H), 2.45-2.38 (m, 1H), 2.34 (t, J=12.0 Hz, 1H); ¹³C NMR (DMSO-d6; 101 MHz) δ: 206.35 (Cq), 165.28 (Cq), 152.82 (Cq), 152.13 (Cq), 146.23 (CH), 139.11 (CH), 134.00 (Cq), 133.75 (Cq), 132.51 (Cq), 129.41 (CH), 122.74 (CH), 117.56 (CH), 117.55 (CH), 112.16 (CH), 55.98 (CH), 43.98 (CH₂), 39.00 (CH₂), 36.59 (CH₂); IR (ATR-Ge v cm⁻¹): 1692 (C═O amide), 1623 (C═O ketone), 1568 (N—H amide), 1479-1457-1421 (C═C arom), 1309 (C—N), 1243 (C—C), 751 (C—H arom); MS (IS) m/z: 337.0 [M+H]⁺.

### 1-(2,6-Dioxo-1,2,3,4,6,10b-hexahydropyrido[2,1-a]isoindol-10-yl)-3-(pyrazin-2-yl)urea (98)

The compound 98 is obtained according to the procedure E from the derivative 26 as a beige solid with a yield of 95%. F>260° C.; IR (ATR-Ge v cm⁻¹): 1689 (C═O amide), 1660 (C═O ketone), 1569 (N—H amide), 1502-1477-1430 (C═C arom), 1304 (C—N), 1143-1065 (C—C); ¹H NMR (DMSO-d6; 400 MHz) δ 10.12 (s, 1H), 9.86 (s, 1H), 8.90 (d, J=1.2 Hz, 1H), 8.37-8.35 (m, 1H), 8.32 (d, J=2.7 Hz, 1H), 8.18 (dd, J=0.9 Hz, 7.9 Hz, 1H), 7.53 (t, J=7.7 Hz, 1H), 7.48 (dd, J=1.0 Hz, J=7.4 Hz, 1H), 5.05 (dd, J=3.9 Hz, J=11.9 Hz, 1H), 4.49-4.44 (m, 1H), 3.48 (td, J=4.4 Hz, J=12.8 Hz, 1H), 3.17 (dd, J=2.7 Hz, J=14.0 Hz, 1H), 2.67-2.57 (m, 1H), 2.44-2.37 (m, 1H), 2.31 (dd, J=2.7 Hz, J=13.6 Hz, 1H); ¹³C NMR (DMSO-d6; 101 MHz) δ 206.34 (Cq), 165.17 (Cq), 151.66 (Cq), 149.14 (Cq), 140.97 (CH), 137.87 (CH), 135.48 (CH), 134.40 (Cq), 133.51 (Cq), 132.62 (Cq), 129.42 (CH), 123.43 (CH), 118.14 (CH), 55.99 (CH), 43.92 (CH₂), 38.99 (CH₂), 36.58 (CH₂); MS (IS) m/z: 338.0 [M+H]⁺.

### 10-(2,4-dioxo-1,2-dihydropyrido[3,4-d]pyrimidin-3(4H)-yl)-1,3,4,10b-tetrahydropyrido[2,1-a]isoindole-2,6-dione (99)

The compound 99 is obtained according to the procedure from the derivative 77 as a white solid with a yield of 76% after flash chromatography on silica gel (CH₂Cl₂/MeOH: 99/1). MP: 230° C.; IR (ATR-Ge v cm⁻¹): 3403 (N—H amide), 1677 (C═O ketone), 1489-1421 (C═C arom), 1272 (C—N); ¹H NMR (DMSO-d6; 400 MHz) δ 12.03 (s, 1H), 8.71 (s, 1H), 8.48 (t, J=4.5 Hz, 1H), 7.87-7.82 (m, 2H), 7.69 (t, J=7.6 Hz, 1H), 7.63 (d, J=7.7 Hz, 1H), 4.93-4.85 (m, 1H), 4.43-4.29 (m, 1H), 3.46-3.37 (m, 1H), 2.57-2.51 (m, 2H), 2.45-2.29 (m, 1H), 2.20 (dd, J=13.9 Hz, 26.1 Hz, 1H); ¹³C NMR (DMSO-d6; 101 MHz) δ 205.6 (Cq), 164.7 (Cq), 161.7 (Cq), 161.1 (Cq), 149.8 (Cq), 149.2 (Cq), 143.0 (CH), 142.8 (Cq), 138.7 (CH), 133.2 (Cq), 132.8 (CH), 130.6 (Cq), 129.8 (CH), 123.7 (CH), 119.6 (CH), 55.5 (CH), 44.0 (CH₂), 38.6 (CH₂), 36.3 (CH₂). MS (IS) m/z: 363.5 [M+H]⁺.

### 1-(2,6-dioxo-1,2,3,4,6,10b-hexahydropyrido[2,1-a]isoindol-10-yl)-3-(3-methylpyridin-2-yl)urea (100)

The compound 100 was obtained according to the procedure D from the compound 45 with a yield of 85% as a white solid. m.p.>260° C.; ¹H NMR (DMSO-d₆, 400 MHz) δₚₚₘ 11.51 (s, 1H), 10.91 (s, 1H), 8.30 (d, J=8.0 Hz, 1H), 8.18 (d, J=7.5 Hz, 1H), 8.06 (d, J=7.5 Hz, 1H), 7.58 (m, 2H), 6.84 (dd, J=7.5 Hz, J=8.0 Hz, 1H), 5.21 (dd, J=2.5 Hz, J=12.5 Hz, 1H), 4.41-4.50 (m, 1H), 3.33-3.54 (m, 2H), 2.39-2.65 (m, 5H), 2.24 (t, J=12.5 Hz, 1H). ¹³C NMR (DMSO-d₆, 100 MHz): 206.3 (Cq), 165.5 (Cq), 153.2 (Cq), 148.4 (Cq), 145.5 (CH), 137.0 (CH), 136.1 (Cq), 133.3 (Cq), 132.9 (Cq), 129.9 (CH), 124.7 (CH), 125.3 (CH), 119.5 (CH), 118.8 (CH), 56.5 (CH), 44.1 (CH₂), 39.3 (CH₂), 36.9 (CH₂), 17.9 (CH₃). IR (ATR-Ge v cm⁻¹): 1684 (C═O amide), 1621 (C═O ketone), 1559 (N—H amide), 1479-1434-1414 (C═C arom), 1329 (C—N), 1289 (C—C), 751 (C—H arom). MS (IS) m/z: 351.0 [M+H]⁺

### 1-(2,6-dioxo-1,2,3,4,6,10b-hexahydropyrido[2,1-a]isoindol-10-yl)-3-(6-methyl-pyridin-2-yl)urea (101)

The compound 101 was obtained according to the procedure D from the compound 46 with a yield of 79% as a white solid. m.p. 226-228° C.; IR (ATR-Ge v cm⁻¹): 1683 (C═O amide), 1621 (C═O ketone), 1566 (N—H amide), 1467-1435 (C═C arom), 1323 (C—N), 1287 (C—C), 752 (C—H arom). ¹H NMR (DMSO-d₆, 400 MHz) δₚₚₘ 10.70 (s, 1H), 9.84 (s, 1H), 8.18 (d, J=7.5 Hz, 1H), 7.63 (dd, J=7.5 Hz, J=8.0 Hz, 1H), 7.46-7.55 (m, 2H), 7.17 (d, J=8.0 Hz, 1H), 6.91 (d, J=7.5 Hz, 1H), 5.08 (dd, J=2.8 Hz, J=12.2 Hz, 1H), 4.48 (dd, J=2.8 Hz, J=12.2 Hz, 1H), 3.45 (td, J=5.0 Hz, J=12.7 Hz, 1H), 3.17 (d, J=5.0 Hz, 1H), 3.11 (dd, J=2.5 Hz, J=12.8 Hz, 1H), 2.54-2.68 (m, 1H), 2.44 (s, 3H), 2.37 (t, J=12.5 Hz, 1H). ¹³C NMR (DMSO-d₆, 100 MHz): 206.8 (Cq), 165.7 (Cq), 155.9 (Cq), 152.7 (Cq), 152.6 (Cq), 139.6 (CH), 134.8 (Cq), 134.2 (Cq), 132.9 (Cq), 129.7 (CH), 124.3 (CH), 118.3 (CH), 117.3 (CH), 109.4 (CH), 55.9 (Cq), 43.6 (CH₂), 39.0 (CH₂), 36.5 (CH₂), 23.5 (CH₃). MS (IS) m/z: 351.0 [M+H]⁺

### 1-(2,6-dioxo-1,2,3,4,6,10b-hexahydropyrido[2,1-a]isoindol-10-yl)-3-(4-methoxyquinolin-2-yl)urea (102)

The compound 102 was obtained according to the procedure D from the compound 48 with a yield of 83% as a white solid. m.p. 223-225° C. IR (ATR-Ge v cm⁻¹): 1683 (C═O amide), 1621 (C═O ketone), 1558 (N—H amide), 1469-1435 (C═C arom), 1329 (C—N), 1289 (C—C), 751 (C—H arom). ¹H NMR (DMSO-d₆, 400 MHz) δₚₚₘ 11.33 (s, 1H), 11.01 (s, 1H), 7.99-8.10 (m, 3H), 7.81 (dd, J=7.5 Hz, J=8.0 Hz, 1H), 7.54-7.57 (m, 3H), 7.01 (s, 1H), 5.18 (dd, J=2.8 Hz, J=12.5 Hz, 1H), 4.43 (dd, J=2.8 Hz, J=12.2 Hz, 1H), 4.12 (s, 3H), 3.50 (td, J=2.5 Hz, J=12.8 Hz, 1H), 3.18 (d, J=12.5 Hz, 1H), 2.52-2.60 (m, 1H), 2.40 (d, J=12.5 Hz, 1H), 0.76 (t, J=12.5 Hz, 1H). ¹³C NMR (DMSO-d₆, 100 MHz): 205.7 (Cq), 165.1 (Cq), 165.0 (Cq), 152.2 (Cq), 151.9 (Cq), 135.7 (Cq), 132.7 (Cq), 132.6 (Cq), 132.3 (CH), 129.3 (2 CH), 125.4 (CH), 124.8 (CH), 122.6 (Cq), 121.9 (CH), 118.9 (CH), 117.5 (Cq), 91.9 (CH), 57.0 (CH), 55.9 (CH₃), 43.4 (CH₂), 38.8 (CH₂), 36.4 (CH₂). HRMS: calc. for C₂₃H₂₁N₄O₄: 417.1563; found 417.1571.

### 1-(2,6-Dioxo-1,2,3,4,6,10b-hexahydropyrido[2,1-a]isoindol-10-yl)-3-(4-hydroxyquinolin-2-yl)urea (103)

The compound 103 was obtained according to the procedure D from the compound 61 with a yield of 80%, as a white solid. m.p.>260° C. IR (ATR-Ge v cm⁻¹): 2872 (O—H), 1695 (C═O amide), 1592 (C═O ketone), 1535 (N—H amide), 1419 (C═C arom), 1348 (C—N), 1296 (C—C), 760 (C—H arom). ¹H NMR (DMSO-d₆, 400 MHz) δₚₚₘ 11.94 (s, 1H), 10.23 (s, 1H), 8.13 (dd, J=8.4 Hz, J=10.0 Hz, 2H), 8.01 (d, J=8.4 Hz, 1H), 7.88 (t, J=8.0 Hz, 1H), 7.54-7.62 (m, 3H), 7.02 (s, 1H), 5.12 (dd, J=4.0 Hz, J=11.6 Hz, 1H), 4.39-4.45 (m, 1H), 3.46 (td, J=4.0 Hz, J=12.8 Hz, 1H), 3.26 (dd, J=4.0 Hz, J=14.0 Hz, 1H), 2.51-2.60 (m, 1H), 2.40 (d, J=13.6 Hz, 1H), 2.23 (t, J=13.6 Hz, 1H). ¹³C NMR (DMSO-d₆, 100 MHz) δₚₚₘ 206.3 (Cq), 165.3 (Cq), 152.4 (2 Cq), 151.2 (Cq), 136.5 (Cq), 133.9 (CH), 133.3 (Cq), 132.7 (Cq), 129.9 (2 CH), 126.4 (Cq), 125.4 (CH), 123.4 (CH), 120.6 (Cq), 119.8 (CH), 118.2 (Cq), 94.6 (CH), 56.4 (CH), 44.0 (CH₂), 39.3 (CH₂), 36.9 (CH₂). HRMS: calc. for C₂₂H₁₉N₄O₄: 403.1406; found 403.1397.

2.6. General Procedure F

Reduction of Ketones into Alcohols (104) to (110).

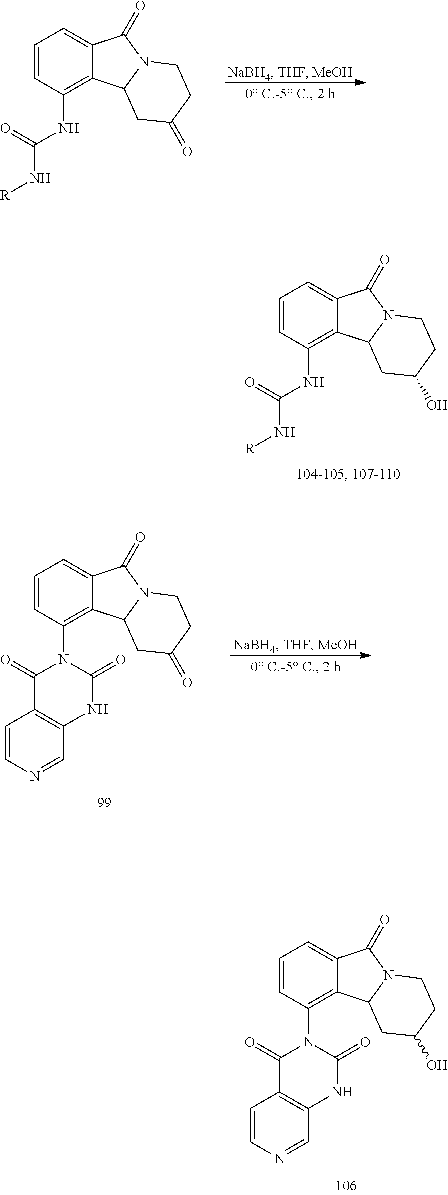

A ketone solution (0.2 mmol) dissolved in a THF:MeOH 1:2 mixture (2 mL/4 mL) is placed at −20° C. with stirring. Sodium borohydride (15 mg, 0.4 mmol, 2.0 equ.) is added portionwise. The reaction medium is stirred for 30 mins and then at a temperature comprised between 0° C. and 5° C. for 2 hours. The solvents are evaporated without heating and the residue is then filtered, successively washed with water, (0.4 mL) and then with an aqueous solution saturated with NaHCO₃ (0.9 mL). After dissolution in dichloromethane (10 mL), the organic phase is dried on MgSO₄, filtered and then evaporated under reduced pressure. The reaction raw product is purified by chromatography on silica gel in order to obtain the expected alcohol as a single diastereoisomer.

### 1-((2S)-2-Hydroxy-6-oxo-1,2,3,4,6,10b-hexahydropyrido[2,1-a]iso-indol-10-yl)-3-(pyridin-2-yl)urea (104)

The compound 104 is obtained according to the procedure F from the derivative 97 as a white solid with a yield of 60% after flash chromatography on silica gel (dichloromethane/methanol 93/7). MP: 220° C.; IR (ATR-Ge v cm⁻¹): 3217 (O—H), 1695 (C═O amide), 1562 (N—H amide), 1512-1478-1430 (C═C arom), 1309 (C—O); ¹H NMR (DMSO-d6; 400 MHz) δ 11.21 (s, 1H), 9.96 (s, 1H), 8.32 (d, J=4.5 Hz, 1H), 8.21 (d, J=7.9 Hz, 1H), 7.81 (t, J=7.1 Hz, 1H), 7.46 (t, J=7.7 Hz, 1H), 7.38 (d, J=7.3 Hz, 1H), 7.33 (d, J=8.4 Hz, 1H), 7.13-7.04 (m, 1H), 4.69 (dd, J=2.8 Hz, J=11.7 Hz, 1H), 4.24 (dd, J=4.1 Hz, J=13.2 Hz, 1H), 3.96 (t, J=10.7 Hz, 1H), 3.60 (s, 1H), 3.08 (td, J=4.1 Hz, J=12.0 Hz, 1H), 2.89 (d, J=11.7 Hz, 1H), 1.95 (d, J=11.6 Hz, 1H), 1.28-1.10 (m, 1H), 0.77 (q, J=12.0 Hz, 1H); ¹³C NMR (DMSO-d6; 101 MHz) δ 64.68 (Cq), 152.77 (Cq), 152.13 (Cq), 146.18 (CH), 139.13 (CH), 134.40 (Cq), 133.82 (Cq), 132.74 (Cq), 128.94 (CH), 122.56 (CH), 117.61 (CH), 117.51 (CH), 112.15 (CH), 66.72 (CH), 56.04 (CH), 38.75 (CH₂), 36.62 (CH₂), 34.50 (CH₂); MS (IS) m/z: 339.0 [M+H]⁺.

### 1-((2S)-2-Hydroxy-6-oxo-1,2,3,4,6,10b-hexahydropyrido[2,1-a]isoindol-10-yl)-3-(pyrazin-2-yl)urea (105)

The compound 105 is obtained according to the procedure F from the derivative 98 as a white solid with a yield of 76% after flash chromatography on silica gel (dichloromethane/methanol 96/4). MP: 250° C.; IR (ATR-Ge v cm⁻¹): 1694 (C═O amide), 1671 (C—N), 1622 (N—H amide), 1568-1545-1500-1485 (C═C arom), 1427 (C—N), 1296 (C—O), 1056 (C—C); ¹H NMR (DMSO-d6; 400 MHz) δ 10.08 (s, 1H), 9.85 (s, 1H), 8.94 (d, J=1.2 Hz, 1H), 8.38-8.32 (m, 1H), 8.31 (d, J=2.7 Hz, 1H), 8.10 (dd, J=0.6 Hz, J=7.8 Hz, 1H), 7.48 (t, J=7.7 Hz, 1H), 7.42 (dd, J=0.8 Hz, J=7.4 Hz, 1H), 5.01 (d, J=4.9 Hz, 1H), 4.65 (dd, J=3.1 Hz, J=11.8 Hz, 1H), 4.24 (dd, J=3.9 Hz, J=13.4 Hz, 1H), 3.95-3.88 (m, 1H), 3.07 (td, J=3.1 Hz, J=13.2 Hz, 1H), 2.78 (d, J=11.8 Hz, 1H), 1.94 (d, J=12.1 Hz, 1H), 1.28-1.08 (m, 1H), 0.75 (q, J=11.7 Hz, 1H); ¹³C NMR (DMSO-d6; 101 MHz) δ 164.59 (Cq), 151.67 (Cq), 149.17 (Cq), 141.14 (CH), 137.90 (CH), 135.39 (CH), 134.97 (Cq), 133.37 (Cq), 132.85 (Cq), 128.96 (CH), 123.27 (CH), 118.07 (CH), 66.69 (CH), 56.05 (CH), 38.71 (CH₂), 36.63 (CH₂), 34.40 (CH₂); MS (IS) m/z: 340.5 [M+H]⁺, 679.5 [2M+H]⁺.

### 3-(2-hydroxy-6-oxo-1,2,3,4,6,10b-hexahydropyrido[2,1-a]isoindol-10-yl)pyrido[3,4-d]pyrimidine-2,4(1H,3H)-dione (106)

The compound 106 is obtained according to the procedure F from the derivative 99 as a beige solid with a yield of 52% after flash chromatography on silica gel (CH₂Cl₂/MeOH: 99/1). IR (ATR-Ge v cm⁻¹): ¹H NMR (CDCl₃; 400 MHz) δ 8.68 (s, 1H), 8.44 (dd, J=5.3 Hz, 6.2 Hz, 1H), 7.96 (t, J=4.0 Hz, 1H), 7.91 (d, J=7.6 Hz, 1H), 7.70 (t, J=7.7 Hz, 1H), 7.62-7.53 (m, 1H), 4.59-4.48 (m, 1H), 4.40 (dd, J=4.7 Hz, 13.5 Hz, 1H), 3.94-3.80 (m, 1H), 3.18-3.04 (m, 1H), 2.27-2.14 (m, 1H), 2.02 (d, J=12.4 Hz, 1H), 1.41-1.24 (m, 1H), 1.13-0.95 (m, 1H).

### 1-(2-Hydroxy-6-oxo-1,2,3,4,6,10b-hexahydropyrido[2,1-a]isoindol-10-yl)-3-(3-methylpyridin-2-yl)urea (107)

The compound 107 was obtained according to the procedure F from the compound 100 after purification by a flash chromatography (CH₂Cl₂/MeOH: 99/1) as a white solid. Yield: 82%. m.p. 204-206° C.; IR (ATR-Ge v cm⁻¹): 2960 (O—H), 1684 (C═O amide), 1584 (N—H amide), 1503-1486-1420 (C═C arom), 1262 (C—O). ¹H NMR (DMSO-d₆, 400 MHz) δₚₚₘ 12.41 (s, 1H), 8.91 (s, 1H), 8.34 (d, J=8.0 Hz, 1H), 8.28 (d, J=7.5 Hz, 1H), 7.73 (d, J=7.5 Hz, 1H), 7.53 (dd, J=7.5 Hz, J=8.0 Hz, 1H), 7.44 (d, J=7.5 Hz, 1H), 7.12 (dd, J=7.5 Hz, J=8.0 Hz, 1H), 5.03 (d, J=5.0 Hz, 1H), 4.77 (dd, J=2.8 Hz, J=12.2 Hz, 1H), 4.30 (dd, J=2.8 Hz, J=12.2 Hz, 1H), 4.00-4.09 (m, 1H), 3.15 (td, J=2.5 Hz, J=12.8 Hz, 1H), 2.96 (d, J=12.5 Hz, 1H), 2.14 (s, 3H), 2.02 (d, J=12.5 Hz, 1H), 1.09-1.25 (m, 1H), 0.85 (q, J=12.5 Hz, 1H). ¹³C NMR (DMSO-d₆, 100 MHz): 165.2 (Cq), 152.8 (Cq), 151.6 (Cq), 143.4 (CH), 140.5 (CH), 134.8 (Cq), 134.3 (Cq), 133.1 (Cq), 129.4 (CH), 122.9 (CH), 121.7 (Cq), 118.3 (CH), 117.9 (CH), 67.2 (CH), 56.4 (CH), 37.1 (CH₂), 35.0 (CH₂), 31.1 (CH₂), 17.4 (CH₃). HRMS: calc. for C₁₉H₂₁N₄O₃: 353.1614; found 353.1616.

### 1-(2-Hydroxy-6-oxo-1,2,3,4,6,10b-hexahydropyrido[2,1-a]isoindol-10-yl)-3-(6-methylpyridin-2-yl)urea (108)

The compound 108 was obtained according to the procedure F from the compound 101 after purification by flash chromatography (CH₂Cl₂/MeOH: 99/1) as a white solid. Yield: 80%. m.p. 240-242° C.; IR (ATR-Ge v cm⁻¹): 3143 (O—H), 1685 (C═O amide), 1589 (N—H amide), 1508-1484-1424 (C═C arom), 1270 (C—O). ¹H NMR (DMSO-d₆, 400 MHz) δₚₚₘ 10.72 (s, 1H), 9.82 (s, 1H), 8.15 (d, J=7.5 Hz, 1H), 7.66 (dd, J=7.5 Hz, J=8.0 Hz, 1H), 7.38-7.49 (m, 2H), 7.16 (d, J=8.0 Hz, 1H), 6.92 (d, J=7.5 Hz, 1H), 4.99 (d, J=5.0 Hz, 1H), 4.71 (dd, J=2.8 Hz, J=12.2 Hz, 1H), 4.26 (dd, J=2.8 Hz, J=12.2 Hz, 1H), 3.84-3.87 (m, 1H), 3.05 (td, J=2.5 Hz, J=12.8 Hz, 1H), 2.75-2.80 (m, 1H), 2.51 (s, 3H), 1.92 (d, J=12.5 Hz, 1H), 1.12-1.18 (m, 1H), 0.76 (q, J=12.5 Hz, 1H). ¹³C NMR (DMSO-d₆, 100 MHz): 164.6 (Cq), 155.3 (Cq), 152.1 (Cq), 152.0 (Cq), 139.0 (CH), 134.6 (Cq), 133.6 (Cq), 132.6 (Cq), 128.7 (CH), 123.4 (CH), 117.6 (CH), 116.7 (CH), 108.8 (CH), 66.6 (CH), 55.9 (CH), 38.2 (CH₂), 36.5 (CH₂), 34.2 (CH₂), 23.7 (CH₃). HRMS: calc. is for C₁₉H₂₁N₄O₃ 353.1614; found 353.1619.

### 1-(2-Hydroxy-6-oxo-1,2,3,4,6,10b-hexahydropyrido[2,1-a]isoindol-10-yl)-3-(4-methoxyquinolin-2-yl)urea (109)

The compound 109 was obtained according to the procedure F from the compound 102 after purification by flash chromatography (CH₂Cl₂/MeOH: 99/1) as a white solid. Yield: 82%. m.p.>260° C.; IR (ATR-Ge v cm⁻¹): 2977 (O—H), 1686 (C═O amide), 1585 (N—H amide), 1512-1479-1414 (C═C arom), 1289 (C—O). ¹H NMR (DMSO-d₆, 400 MHz) δₚₚₘ 11.97 (s, 1H), 10.15 (s, 1H), 8.20 (d, J=7.5 Hz, 1H), 8.03 (d, J=8.0 Hz, 1H), 7.88 (d, J=8.0 Hz, 1H), 7.73 (dd, J=7.5 Hz, J=8.0 Hz, 1H), 7.41-7.53 (m, 3H), 6.87 (s, 1H), 4.89 (d, J=12.5 Hz, 2H), 4.26 (dd, J=2.8 Hz, J=12.5 Hz, 1H), 4.02 (s, 3H), 3.77-3.88 (m, 1H), 3.14 (td, J=2.8 Hz, J=12.5 Hz, 1H), 2.79 (d, J=12.5 Hz, 1H), 1.94 (d, J=12.5 Hz, 1H), 1.03-1.23 (m, 1H), 0.76 (q, J=12.5 Hz, 1H). ¹³C NMR (DMSO-d₆, 100 MHz): 164.5 (Cq), 162.9 (Cq), 153.6 (Cq), 152.2 (Cq), 145.6 (Cq), 134.9 (Cq), 133.4 (Cq), 132.7 (Cq), 130.7 (CH), 128.8 (CH), 126.0 (CH), 123.9 (CH), 123.7 (CH), 121.3 (CH), 118.0 (Cq), 117.8 (CH), 91.8 (CH), 66.6 (CH), 56.1 (CH), 56.0 (CH₃), 38.0 (CH₂), 36.4 (CH₂), 34.4 (CH₂). HRMS: calc. for C₂₃H₂₃N₄O₄ 419.1719; found 419.1728.

### 1-(2-Hydroxy-6-oxo-1,2,3,4,6,10b-hexahydropyrido[2,1-a]isoindol-10-yl)-3-(4-hydroxyquinolin-2-yl)urea (110)

The compound 110 was obtained according to procedure F from the compound 103 after purification by flash chromatography (CH₂Cl₂/MeOH: 99/1) as a white solid. Yield: 82%. m.p.>260° C.; IR (ATR-Ge v cm⁻¹): 3343 (O—H), 1680 (C═O amide), 1564 (C═O ketone), 1504 (N—H amide), 1425 (C═C arom), 1353 (C—N), 1294 (C—C), 754 (C—H arom). ¹H NMR (DMSO-d₆, 400 MHz) δₚₚₘ 13.66 (s, 1H), 8.98 (s, 1H), 8.27 (d, J=8.0 Hz, 1H), 8.96 (d, J=8.4 Hz, 1H), 7.46 (t, J=8.4 Hz, 2H), 7.42 (d, J=7.6 Hz, 1H), 7.31-7.35 (m, 2H), 6.96 (t, J=7.6 Hz, 1H), 5.60 (s, 1H), 4.87-4.90 (m, 2H), 4.24 (dd, J=3.6 Hz, J=12.8 Hz, 1H), 3.83-3.90 (m, 1H), 3.15 (td, J=3.6 Hz, J=12.8 Hz, 1H), 3.93 (d, J=12.0 Hz, 1H), 1.93 (d, J=12.8 Hz, 1H), 1.06-1.17 (m, 1H), 0.73 (q, J=12.0 Hz, 1H). ¹³C NMR (DMSO-d₆, 100 MHz) δₚₚₘ 174.6 (Cq), 164.9 (Cq), 154.9 (Cq), 153.8 (Cq), 147.5 (Cq), 134.8 (Cq), 134.2 (Cq), 132.5 (Cq), 128.7 (CH), 128.0 (CH), 126.8 (Cq), 124.7 (CH), 123.9 (CH), 123.0 (CH), 119.1 (CH), 116.6 (CH), 93.9 (CH), 66.6 (CH), 56.4 (CH), 38.0 (CH₂), 36.6 (CH₂), 34.6 (CH₂). HRMS: calc. for C₂₂H₂₀N₄O₄Na 427.1382; found 427.1378.

2.7. General Procedure G

Synthesis of the ureas (113) to (117) Based on Pyridopyrimidinediones from the Amines (5) or (10).

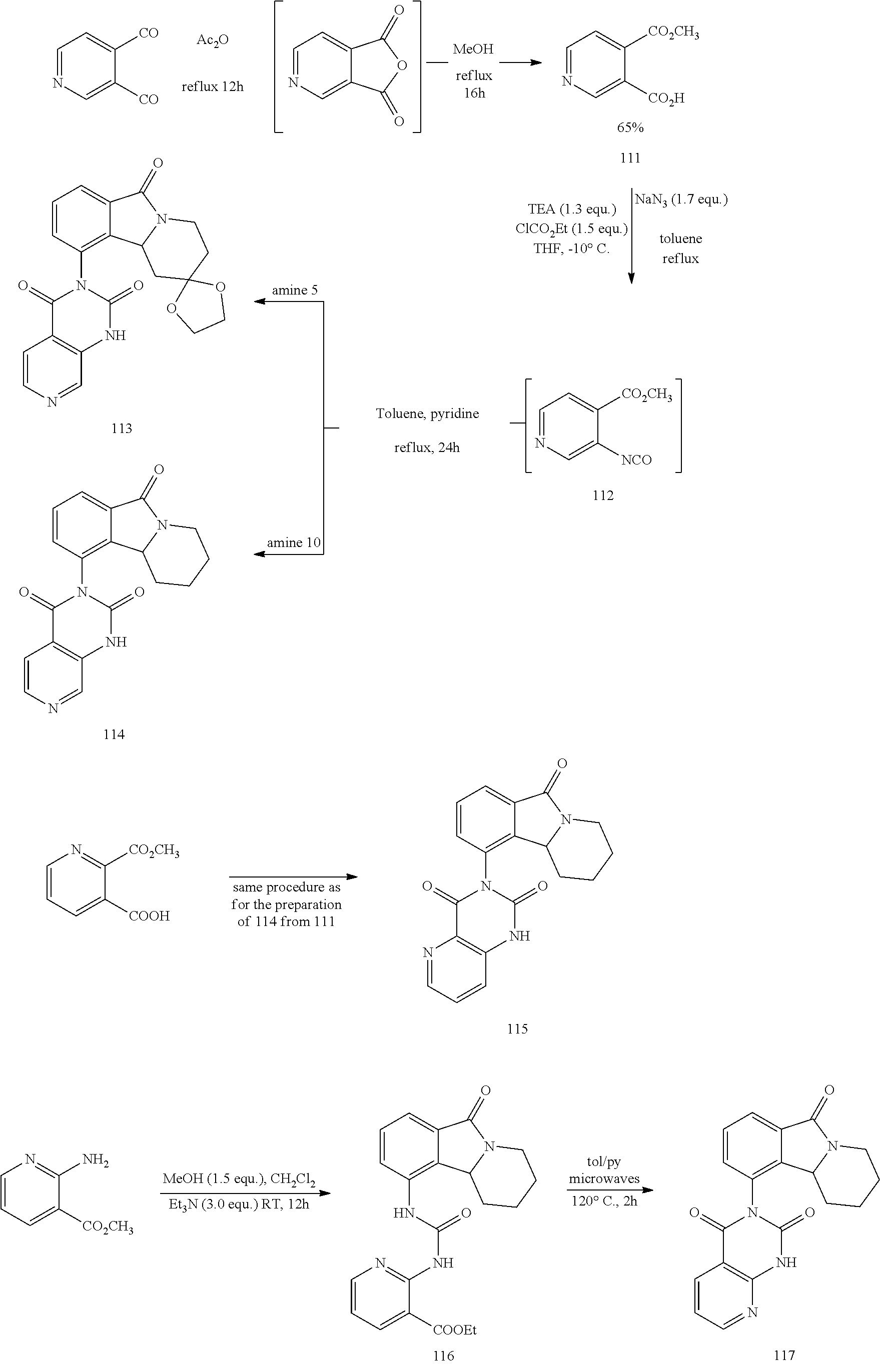

Synthesis of the hemi-ester 111: Pyridine-3,4-dicarboxylic acid (2.0 g, 11.05 mmol) is heated in acetic anhydride (15 mL) to 100° C. for 12 hours. After cooling, the solvent is evaporated and the reaction raw product is placed with reflux of methanol (20 mL) for 16 hours. After evaporation of the solvent, recrystallisation from 10 mL of methanol leads to the monoester 111 with a yield of 65%, which is used without any other purification in the following step.

### 4-(methoxycarbonyl)nicotinic acid (111)

The compound III is obtained as a brown solid with a yield of 67% after recrystallisation from methanol. IR (ATR-Ge v cm⁻¹): ¹H NMR (DMSO-d6; 400 MHz) δ 13.79 (s, 1H), 9.03 (s, 1H), 8.86 (d, J=4.9 Hz, 1H), 7.62 (d, J=4.9 Hz, 1H), 3.84 (s, 3H); ¹³C NMR (DMSO-d6; 101 MHz) δ 166.9 (Cq), 166.1 (Cq), 153.1 (CH), 150.2 (CH), 140.4 (Cq), 125.2 (Cq), 121.6 (CH), 52.9 (CH₃). MS (IS) m/z: 182.5 [M+H]⁺

### Synthesis of the Isocyanate (112)

In an argon atmosphere, triethylamine (0.24 mL, 1.3 equ.) is added to a stirred solution of 111 (1.35 mmol, 1.0 equ.) in anhydrose THF (10 mL). The solution was cooled down to −10° C. and methyl chloroformate (0.2 mL, 1.5 equ.) was added dropwise. The resulting mixture was stirred for one hour at 10° C., and then sodium nitrite was slowly added (149 mg, 1.7 equ.) in 2 mL of water. After one hour at −10° C., the reaction was finished and the reaction mixture was extracted with ethyl acetate (3×10 mL). The combined organic layers were dried on MgSO₄, filtered and evaporated. The obtained acyl nitrite was again dissolved in dry toluene (10 mL) and refluxed with ED by heating for one hour under an Ar atmosphere, in order to obtain the corresponding isocyanate 112 which was used without any additional purification.

General procedure G: Synthesis of Pyridopyrimidine Diones 113-117.

Under an argon atmosphere, a solution of the isocyanate 112 (1.0 mmol, 1.0 equ.) was added to a solution of isoindolonic amine (1 mmol, 5 or 10) in 5 mL of pyridine. The mixture was refluxed for 24 hours, cooled and concentrated in vacuo. The raw residue was purified by flash chromatography in order to obtain the pyridopyrimidine-diones.

### 3-(6′-oxo-3′,4′,6′,10b′-tetrahydro-1′H-spiro[[1,3]dioxolane-2,2′-pyrido[2,1-a]isoindole]-10′-yl)pyrido[3,4-d]pyrimidine-2,4(1H,3H)-dione (113)

The compound 113 is obtained according to the procedure G from the amine 5 and 111 as a white solid with a yield of 67% after flash chromatography on silica gel (CH₂Cl₂/MeOH: 99/1). MP: 204° C.; IR (ATR-Ge v cm⁻¹); ¹H NMR (DMSO-d6; 400 MHz) δ 12.06 (s, 1H), 8.70 (s, 1H), 8.49 (d, J=4.7 Hz, 1H), 7.86 (dd, J=2.7 Hz, 4.8 Hz, 1H), 7.81 (d, J=7.1 Hz, 1H), 7.68-7.61 (m, 2H), 4.46 (m, 1H), 4.24 (dd, J=5.1 Hz, 13.1 Hz, 1H), 3.88-3.68 (m, 4H), 3.15-3.00 (m, 1H), 1.85-1.74 (m, 2H), 1.50-1.45 (m, 1H), 1.38-1.20 (m, 1H); ¹³C NMR (DMSO-d6; 101 MHz) δ 164.1 (Cq), 161.6 (Cq), 161.0 (Cq), 149.7 (Cq), 149.1 (Cq), 143.3 (CH), 142.3 (Cq), 138.8 (CH), 133.4 (Cq), 132.6 (CH), 130.5 (Cq), 129.5 (CH), 123.6 (CH), 119.6 (CH), 106.4 (Cq), 64.0 (CH₂), 63.8 (CH₂), 55.5 (CH), 38.2 (CH₂), 36.0 (CH₂), 32.7 (CH₂). MS (IS) m/z: 407.5 [M+H]⁺.

### 3-(6-oxo-1,2,3,4,6,10b-hexahydropyrido[2,1-a]isoindol-10-yl)pyrido[3,4-d]-pyrimidine-2,4(1H,3H)-dione (114)

The compound 114 is obtained according to the procedure G from the amine 10 and from the isocyanate 112 as a grey solid with a yield of 52% after flash chromatography on silica gel (CH₂Cl₂/MeOH: 99/1). F>260° C.; IR (ATR-Ge v cm⁻¹): 1656 (C═O amide), 1590-1416 (C═C arom), 1260 (C—N), 1153 (C—C); ¹H NMR (DMSO-d6; 400 MHz) δ 11.97 (d, J=9.9 Hz, 1H), 8.69 (d, J=3.6 Hz, 1H), 8.47 (d, J=5.0 Hz, 1H), 7.84 (dd, J=2.9 Hz, 5.0 Hz, 1H), 7.79 (d, J=7.0 Hz, 1H), 7.69-7.54 (m, 2H), 4.35-4.27 (m, 1H), 4.21 (d, J=9.9 Hz, 1H), 2.98-2.91 (m, 1H), 2.02-1.88 (m, 1H), 1.68 (d, J=12.6 Hz, 2H), 1.50 (dd, J=13.1 Hz, 26.3 Hz, 1H), 1.28-1.11 (m, 1H), 0.99-0.79 (m, 1H); ¹³C NMR (DMSO-d6; 101 MHz) δ 163.9 (Cq), 161.7 (Cq), 161.0 (Cq), 149.8 (Cq), 149.2 (Cq), 143.0 (CH), 138.7 (CH), 135.2 (Cq), 133.5 (Cq), 132.3 (CH), 130.6 (Cq), 129.2 (CH), 123.3 (CH), 119.6 (CH), 57.2 (CH), 39.0 (CH₂), 30.1 (CH₂), 24.8 (CH₂), 22.7 (CH₂). MS (IS) m/z: 349.0 [M+H]⁺, 697.5 [2M+H]⁺.

### 3-(6-Oxo-1,2,3,4,6,10b-hexahydro-pyrido-pyrido[2,1-a]isoindol-10-yl)-1H-pyrid o[3,2-d]pyrimidine-2,4-dione (115)

The compound 115 was obtained according to procedure G from the corresponding isocyanate and the amine 10 after purification by flash chromatography (CH₂Cl₂/MeOH: 99/1) as a beige solid. Yield: 66%. m.p. 232° C.; IR (ATR-Ge v cm⁻¹): 2929 (Csp3-H), 1669 (C═O amide), 1597-1457 (C═C arom), 1286 (C—N); ¹H NMR (DMSO-d6; 400 MHz) δ 11.80 (d, J=9.8 Hz, 1H), 8.55 (d, J=2.5 Hz, 1H), 7.79 (d, J=7.2 Hz, 1H), 7.75-7.68 (m, 2H), 7.67-7.54 (m, 2H), 4.46-4.27 (m, 1H), 4.21 (d, J=10.5 Hz, 1H), 2.95 (t, J=12.9 Hz, 1H), 2.09-1.83 (m, 1H), 1.69-1.66 (m, 2H), 1.50 (dd, J=24.2 Hz, 11.8 Hz, 1H), 1.32-1.10 (m, 1H), 0.98-0.85 (m, 1H); ¹³C NMR (DMSO-d6; 101 MHz) δ 164.0 (Cq), 160.3 (Cq), 149.5 (Cq), 148.9 (Cq), 145.1 (CH), 143.3 (Cq), 137.5 (Cq), 133.5 (Cq), 132.4 (CH), 131.0 (Cq), 129.2 (2CH), 124.0 (CH), 123.2 (CH), 57.2 (CH), 39.0 (CH₂), 30.1 (CH₂), 24.9 (CH₂), 22.8 (CH₂); HRMS: calc. for C₁₉H₁₆N₄O₃Na 371.1120; found 371.1110.

### 2-[3-(6-Oxo-1,2,3,4,6,10b-hexahydro-pyrido[2,1-a]isoindol-10-yl)-ureido]-nicotinic acid methyl ester (116)

At 0° C. under Ar, bis(trichloromethyl)carbonate (297 mg; 1.0 equ.) was added to a stirred solution of 10 (198 mg; 1 mmol) and of Et₃N (0.28 mL; 2 equ.) in dry CH₂Cl₂ (10 mL), and the reaction medium was then gradually brought to reflux. After 5 hours of reflux, the reaction medium was cooled. A solution of 2-amino-nicotinic acid methyl ester (152 mg; 1.0 equ.) in dry pyridine (1.6 mL) was added to a cooled solution of the raw isocyanate in CH₂Cl₂. The reaction medium was stirred at room temperature for 18 hours and then diluted with water and extracted with AcOEt. The organic phase was washed with water, dried on MgSO₄ and evaporated in vacuo. The residue was purified by chromatography on silica gel (methanol/dichloromethane:1/99). The compound 116 was obtained as a beige solid. Yield: 40%. m.p. 205° C.; IR (ATR-Ge v cm⁻¹): 3269 (N—H amide), 2921 (Cₛₚ₃—H), 1727 (C═O ester), 1680 (C═O amide), 1589-1484 (C═C arom), 1418 (C—O), 1253 (C—N); ¹H NMR (CDCl₃; 400 MHz) δ 11.85 (s, 1H), 10.41 (s, 1H), 8.50-8.28 (m, 2H), 8.17 (d, J=8.0 Hz, 1H), 7.60 (d, J=7.4 Hz, 1H), 7.43 (t, J=7.8 Hz, 1H), 7.04 (dd, J=7.8 Hz, 5.0 Hz, 1H), 4.54-4.44 (m, 2H), 3.96 (s, 3H), 3.00 (td, J=13.0 Hz, 3.3 Hz, 1H), 2.71 (dd, J=12.8 Hz, 2.5 Hz, 1H), 2.01 (d, J=13.2 Hz, 1H), 1.93-1.77 (m, 1H), 1.76-1.58 (m, 1H), 1.52-1.33 (m, 1H), 1.07 (qd, J=13.0 Hz, 3.2 Hz, 1H); ¹³C NMR (CDCl₃; 101 MHz) δ 166.3 (Cq), 166.1 (Cq), 153.5 (Cq), 151.8 (Cq), 150.4 (CH), 141.7 (CH), 135.7 (Cq), 133.5 (Cq), 133.3 (Cq), 129.3 (CH), 124.0 (CH), 119.4 (CH), 116.8 (CH), 110.0 (Cq), 58.6 (CH), 53.1 (CH₃), 39.9 (CH₂), 30.4 (CH₂), 25.5 (CH₂), 24.1 (CH₂); HRMS: calc. for C₂₀H₂₁N₄O₄381.1563; found 381.1550.

### 3-(6-Oxo-1,2,3,4,6,10b-hexahydro-pyrido[2,1-a]isoindol-10-yl)-1H-pyrido[2,3-d]pyrimidine-2,4-dione (117)

The compound 116 (60 mg; 380.41 g/mol; 0.16 mmol) was cyclized by irradiation of microwaves in a toluene/pyridine solution (1/1; 2 mL/2 mL) at 120° C. for 2 hours. The residue was purified by chromatography on silica gel (methanol/dichloromethane:1/99). The compound 117 was obtained as a beige solid.

Yield: 57%. m.p.>290° C.; IR (ATR-Ge v cm⁻¹): 2928 (Cₛₚ₃—H), 1678 (C═O amide), 1397 (C—N); ¹H NMR (DMSO-d6; 400 MHz) δ 12.19 (s, 1H), 8.71 (dd, J=4.8 Hz, 1.8 Hz, 1H), 8.36-8.33 (m, 1H), 7.83-7.74 (m, 1H), 7.68-7.54 (m, 2H), 7.35-7.32 (m, 1H), 4.36-4.27 (m, 1H), 4.21 (dd, J=12.8 Hz, 3.2 Hz, 1H), 3.01-2.88 (m, 1H), 1.95 (dd, J=35.9 Hz, 9.8 Hz, 1H), 1.75-1.66 (m, 2H), 1.56-1.46 (m, 1H), 1.34-1.10 (m, 1H), 0.97-0.82 (m, 1H); ¹³C NMR (DMSO-d6; 101 MHz) δ 164.0 (Cq), 162.0 (Cq), 155.1 (CH), 151.5 (Cq), 150.2 (Cq), 143.5 (Cq), 137.2 (CH), 133.6 (Cq), 132.5 (CH), 130.8 (Cq), 129.2 (CH), 123.3 (CH), 119.3 (CH), 109.8 (Cq), 57.2 (CH), 39.0 (CH₂), 30.3 (CH₂), 24.9 (CH₂), 22.7 (CH₂); HRMS: calc. for C₁₉H₁₇N₄O₃: 349.1301; found: 349.1290.

### 3-[3-(6-Oxo-1,2,3,4,6,10b-hexahydro-pyrido[2,1-a]isoindol-10-yl)-ureido]-pyrazole-1-carboxylic acid butyl ester (118)

At 0° C. under Ar, bis(trichloromethyl)carbonate (207 mg; 1.0 equ.) was added to a stirred solution of 10 (140 mg; 0.7 mmol) and of Et₃N (0.2 mL; 2.0 equ.) in dry CH₂Cl₂ (10 mL), and the reaction mixture was then gradually brought to reflux. After 5 hours of reflux, the reaction mixture was cooled. A solution of 3-amino-pyrazole-1-carboxylic acid tertio-butyl ester methyl ester (128 mg; 1.0 equ.) in dry pyridine (1.2 mL) was added to a cooled solution of the raw isocyanate in CH₂Cl₂. The reaction mixture was stirred at room temperature for 18 hours and then diluted with water and extracted with AcOEt. The organic phase was washed with water, dried on MgSO₄ and evaporated in vacuo. The residue was purified by chromatography on silica gel (methanol/dichloromethane:1/99). The compound 118 was obtained as a yellow solid. Yield: 56%. m.p. 136° C.; IR (ATR-Ge v cm⁻¹): 3269 (N—H amide), 2937 (Cₛₚ₃—H), 1668 (C═O amide), 1575-1484 (C═C arom), 1320 (C—O), 1287 (C—N), 1141 (C—C); ¹H NMR (CDCl₃; 400 MHz) δ 10.32 (s, 1H), 9.59 (s, 1H), 7.99 (s, 1H), 7.91 (d, J=2.9 Hz, 1H), 7.63 (d, J=7.4 Hz, 1H), 7.42 (t, J=7.8 Hz, 1H), 6.38 (s, 1H), 4.64 (d, J=9.8 Hz, 1H), 4.49 (dd, J=13.0 Hz, 3.8 Hz, 1H), 2.99 (t, J=11.8 Hz, 1H), 2.65 (s, 1H), 2.18 (s, 1H), 1.85 (d, J=13.0 Hz, 1H), 1.76 (d, J=11.8 Hz, 1H), 1.62 (s, 9H), 1.45-1.27 (m, 1H), 0.98 (qd, J=12.7 Hz, 3.2 Hz, 1H); ¹³C NMR (CDCl₃; 101 MHz) δ 166.2 (2Cq), 153.1 (Cq), 152.3 (Cq), 133.7 (Cq), 133.2 (2Cq), 131.9 (2CH), 129.0 (2CH), 119.8 (CH), 85.7 (Cq), 58.6 (CH), 40.1 (CH₂), 30.3 (CH₂), 28.2 (3CH₃), 25.6 (CH₂), 23.5 (CH₂); HRMS: calc. for C₂₁H₂₅N₅O₄Na 434.1804; found 434.1791.

### 1-(6-Oxo-1,2,3,4,6,10b-hexahydro-pyrido[2,1-a]isoindol-10-yl)-3-(1H-pyrazol-3-yl)-urea (119)

The mixture of 118 (60 mg; 0.146 mmol) and of NaOH (1 M; 20 mL) was stirred at room temperature for one night. The reaction mixture was extracted with hot ethyl acetate (3×20 mL). The combined organic extracts were dried on MgSO₄. Removal of the solvents provided the residue which was purified by flash chromatography. The compound 119 was obtained as a white solid. Yield: 66%. m.p. 185 C; IR (ATR-Ge v cm⁻¹): 3342 (N—H amine), 2919 (Cₛₚ₃—H), 1707 (C═O amide), 1600-1547 (C═C arom), 1266 (C—N); ¹H NMR (DMSO-d₆. MHz) δ 12.33 (s, 1H), 9.40 (s, 1H), 9.06 (s, 1H), 8.12 (d, J=7.9 Hz, 1H), 7.63 (s, 1H), 7.42 (t, J=7.7 Hz, 1H), 7.35 (d, J=7.2 Hz, 1H), 6.20 (s, 1H), 4.48 (dd, J=11.5 Hz, 3.2 Hz, 1H), 4.26 (dd, J=13.1 Hz, 4.3 Hz, 1H), 3.01 (td, J=12.7 Hz, 3.0 Hz, 1H), 2.66 (d, J=10.7 Hz, 1H), 1.90 (d, J=13.2 Hz, 1H), 1.83-1.56 (m, 2H), 1.34-1.16 (m, 1H), 0.84 (qd, J=12.8 Hz, 3.2 Hz, 1H); ¹³C NMR (DMSO-d₆; 101 MHz) δ 164.6 (Cq), 151.8 (Cq), 147.9 (Cq), 134.7 (Cq), 134.4 (Cq), 132.8 (Cq), 129.2 (CH), 128.7 (CH), 122.2 (CH), 117.0 (CH), 94.1 (CH), 57.4 (CH), 39.1 (CH₂), 29.9 (CH₂), 25.0 (CH₂), 23.0 (CH₂); MS (IS) m/z: 312.0 [M+H]⁺; 623.5 [2M+H]⁺

Biological Results

1. Dosage Methods

The protein kinase inhibition activities of the compounds of the invention are tested according to the following general procedure:

Buffer Solutions

Buffer A: 10 mM mgCl₂, 1 mM EGTA, 1 mM DTT, 25 mM Tris-HCl pH 7.5 and 50 μg heparin/mL.

Buffer C: 60 mM β-glycerophosphate, 15 mM p-nitrophenyl-phosphate, 25 mM Mops (pH 7.2), 5 mM EGTA, 15 mM mgCl₂, 1 mM DTT, 1 mM sodium vanadate and 1 mM phenylphosphate.

Preparations of the Kinases and Dosages

The kinase activities are assayed in the A or C buffers, at 30° C., at a final concentration of ATP of 15 μM. The values of the blanks were subtracted and the activities are expressed as a % of the maximum activity, i.e. in the absence of inhibitors. The controls were made with suitable dilutions of DMSO.

The CDK1/cyclin B (native sea star ovocytes in phase M) and CDK5/D25 (human, recombinant) were prepared as described earlier (Leclerc, S.; Garnier, M.; Hoessel, R.; Marko, D.; Bibb, J. A.; Snyder, G. L.; Greengard, P.; Biernat, J.; Mandelkow, E.-M.; Eisenbrand, G.; Meijer, L. Indirubins inhibit glycogen synthase kinase-3β and CDK5/p25, two kinases involved in abnormal tau phosphorylation in Alzheimer's disease—A property common to most CDK inhibitors? J. Biol. Chem. 2001, 276, 251-260; Bach S, Knockaert M, Reinhardt J, Lozach O, Schmitt S, Baratte B et al. (2005). Roscovitine targets, protein kinases and pyridoxal kinase. J Biol Chem 280: 31208-31219). Their kinase activity was analysed in buffer C, with 1 mg of histone H1/mL, in the presence of 15 μM de [γ-³³P] ATP (3.000 Ci/mmol.; 10 mCi/mL) in a final volume of 30 μL. After 30 mins of incubation at 30° C., 25 μL of supernatant aliquots were deposited on phosphocellulose Whatman P81 paper pieces of 2.5×3 cm, and after 20 seconds, the filters were washed five times (for at least 5 minutes each time) in a solution of 10 mL of phosphoric acid/1 litre of water. The wet filters were counted in the presence of 1 mL of scintillation fluid ACS (Amersham).

GSK-3αβ (native pig brain) was assayed, as described for CDK1 but in the buffer A and by using a specific substrate of GSK-3 (GS-1: YRRAAVPPSPSLSRHSSPHQSpEDEEE) (Sp represents a phosphorylated serine) (Primot, A., Baratte, B., Gompel, M., Borgne, A., Liabeuf, S., Romette, J. L., Costantini, F. and Meijer, L., 2000. Purification of GSK-3 by affinity chromatography on immobilized axin. Protein Expr. & Purif. 20 (3), 394-404). GS-1a was synthesized by Millegen (Labege, France).

DYRK1A (human, recombinant, expressed in E. coli as a fusion protein GST) was purified by affinity chromatography on glutathione-agarose beads and measured in the buffer A (+0.5 mg of bovine albumin serum/mL) with the Woodtide substrate (1.5 μg/dosage).

In Vivo Assays on Human Cells (Huh7, Caco, MDA-MB 231, HCT 116, PC3, NCl, Fibroblast)

Cytotoxicity: This method is based on an automated imaging analysis. 4.10³ cells were cultivated on 96-well plates and left for 24 hours so as to bind, to spread and proliferate.

These cells were then exposed for 24 h to 48 h to increasing concentrations of the compounds of the invention, from 0.1 to 25 μM in a final volume of 80 μL of culture medium. The cells were then fixed with a 4% paraformaldehyde solution and the nuclei were stained with Hoechst 3342 and counted according to an automated imaging quantification system.

All the cell lines were cultivated in a DMEM or RPMI (Invitrogen) medium. All these media were completed with antibiotics (penicillin-streptomycin) (Lonza) and 10% by volume of fetal calf serum (Invitrogen). The cells were cultivated at 37° C. with 5% of CO₂. The treatments with the molecules to be tested were carried out with increasing concentrations. The control experiments were also carried out by using suitable dilutions of DMSO (maximum 1% DMSO). Cell viability was determined by measuring the reduction of MTS as described in the article of Ribas J, Boix J. (2004). Cell differentiation caspase inhibition and macromolecular synthesis blockage but not BCL-2 or BCL-XL proteins protect SH-SY5Y cells from apoptosis triggered by two CDK inhibitory drugs. Exp. Cell Res., 295: 9-24.

2. Results

The obtained results are indicated in the tables hereafter.

| | | IC₅₀ μM | | | | | | |
|---|---|---|---|---|---|---|---|---|
| | | Human cells | | | | | | |
| | | | | MDA- | HCT | | | |
| | | Huh7 | Caco | MB 231 | 116 | PC3 | NCI | |
| Number | Species | (liver) | (colon) | (breast) | (colon) | (prostate) | (lung) | fibroblast |
| 16 | 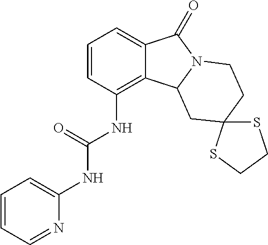 | 15 | 15 | 20 | 10 | 6 | 25 | >25 |
| 17 | 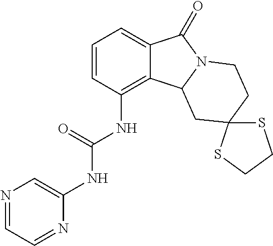 | 0.1 | 0.5 | 0.15 | 0.1 | 0.6 | 0.4 | 0.8 |
| 18 | 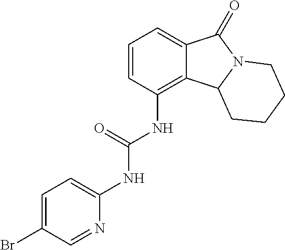 | >25 | >25 | >25 | >25 | >25 | >25 | >25 |
| 19 | 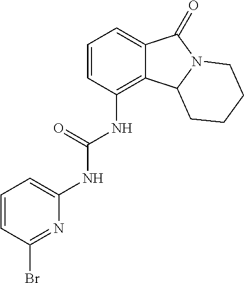 | >25 | >25 | >25 | >25 | >25 | >25 | >25 |
| 20 | 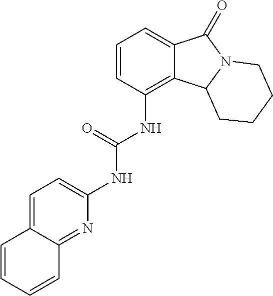 | 0.01 | <0.01 | <0.03 | 0.01 | 0.01 | 0.08 | 0.3 |
| 21 | 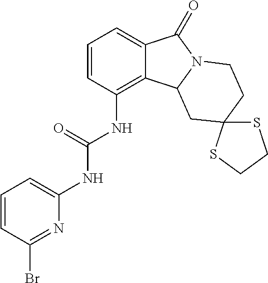 | 2 | 3 | 4 | 2.5 | 4 | 6 | 9 |
| 22 | 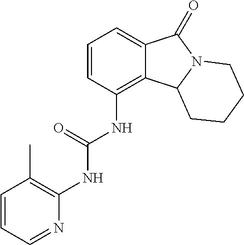 | 0.04 | 0.07 | 0.15 | 0.01 | 0.1 | 0.15 | 0.4 |
| 23 | 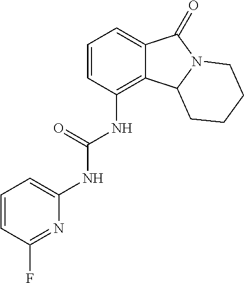 | 0.1 | 0.03 | 0.03 | <0.01 | 0.03 | 0.3 | 0.3 |
| 24 | 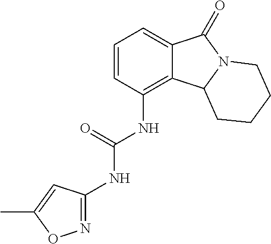 | 3 | 3 | 3 | 2 | 6 | 10 | 12 |
| 25 | 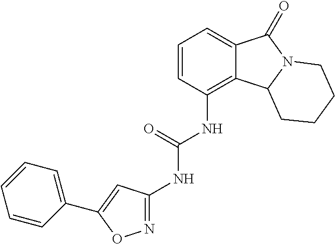 | 0.25 | 0.1 | 0.2 | 0.03 | 0.1 | 1.5 | 0.1 |
| 26 | 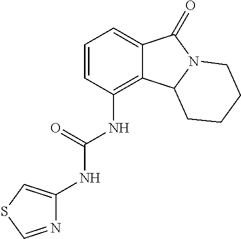 | 1.2 | 0.8 | 0.4 | 0.8 | 2 | 5 | 4 |
| 27 | 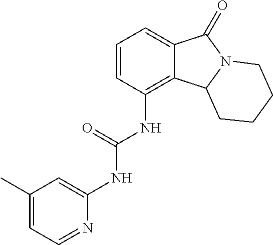 | 0.01 | 0.01 | <0.01 (0.01) | 0.01 | 0.01 | 0.03 | 0.1 |
| 28 | 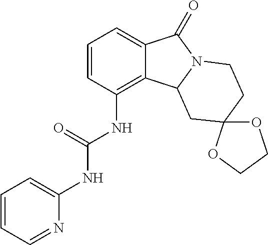 | 1 | 0.6 | 1 | 0.6 | 1.5 | 4 | 3 |
| 29 | 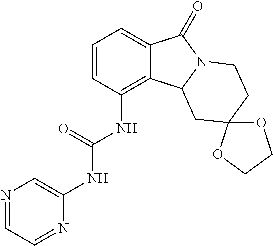 | 3 | 5 | 3 | 1.5 | 5 | 8 | 10 |
| 30 | 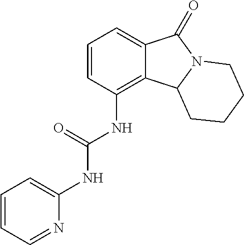 | 0.03 | 0.1 | 0.05 | 0.08 | 0.1 | 0.2 | 0.2 |
| 31 | 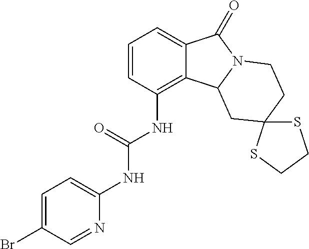 | 0.8 | 5 | 4 | 1.2 | 3 | 1.5 | 8 |
| 32 | 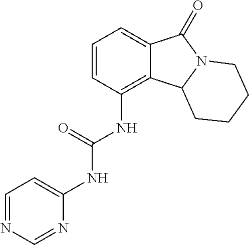 | | | | | | | |
| 33 | 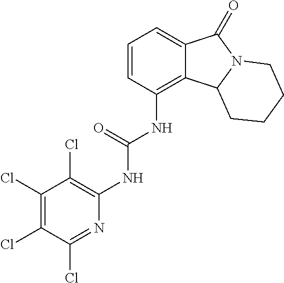 | 0.25 | 1.5 | 0.5 | 0.5 | 1 | 0.8 | 0.5 |
| 34 | 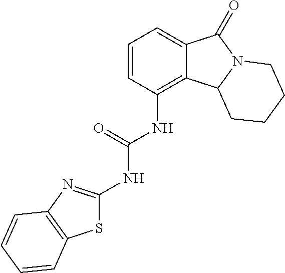 | 0.6 | 0.7 | 1.5 | 0.5 | 1 | 4 | 2 |
| 35 | 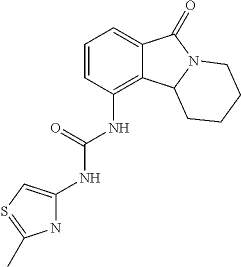 | 0.9 | 0.7 | 1.5 | 0.6 | 1.5 | 3 | 0.4 |
| 36 | 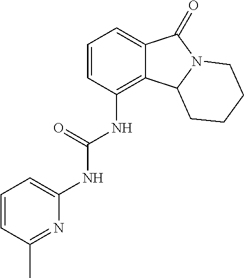 | 0.04 | 0.01 | <0.1 | 0.04 | 0.04 | 0.15 | 0.04 |
| 37 | 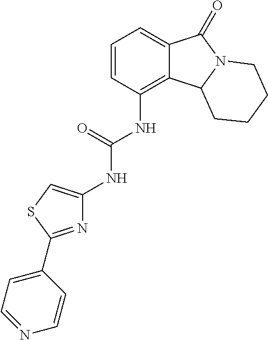 | 2 | 1.5 | 1.5 | 0.7 | 1 | 4 | 3 |
| 38 | 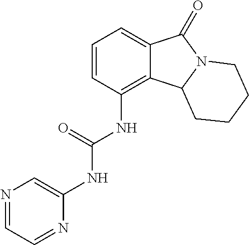 | 0.2 | 1 | 0.2 | 0.1 | 0.3 | 0.8 | 0.8 |
| 39 | 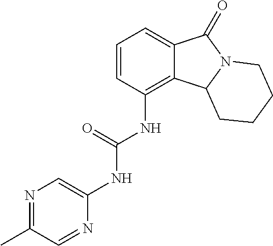 | 0.15 | 0.4 | 0.15 | 0.15 | 0.25 | 0.5 | 0.3 |
| 40 | 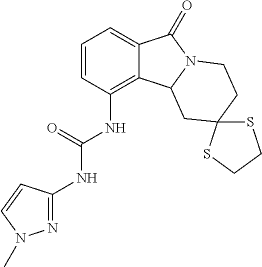 | | | | | | | |
| 41 | 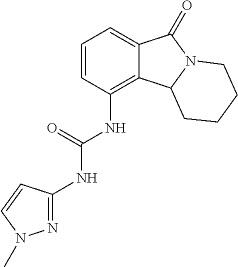 | | | | | | | |
| 42 | 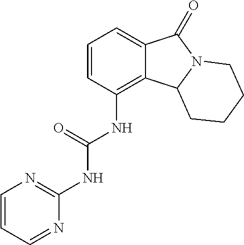 | 0.6 (60%) | 2 | 4 | 4 | 2 | 8 | 2.5 (50%) |
| 43 | 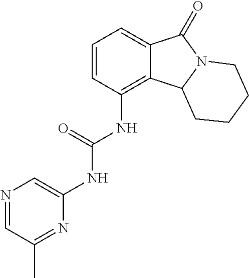 | 0.4 | 0.8 | 0.5 | 0.25 | 0.4 | 0.5 | 0.8 |
| 44 | 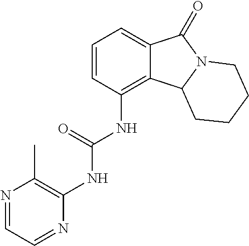 | 0.6 | 0.6 | 1.5 | 0.5 | 0.9 | 1 | 1.5 |
| 45 | 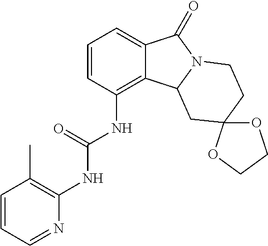 | >25 | >25 | >25 | 25 | >25 | >25 | >25 |
| 46 | 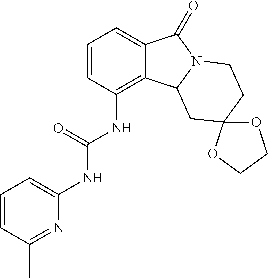 | 12 | 10 | 15 | 10 | 12 | 12 | 20 |
| 47 | 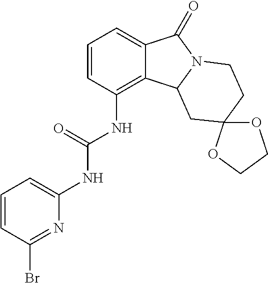 | 8 | 12 | 20 | 6 | 6 | 20 | 20 |
| 48 | 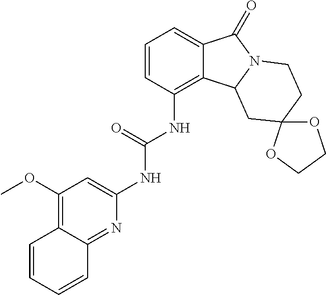 | 8 | >25 | 4 | 8 | 8 | 20 | 20 |
| 49 | 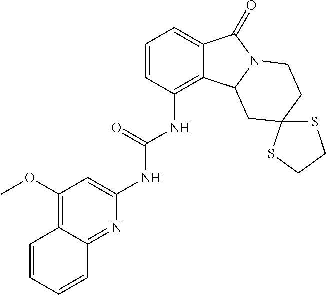 | 5 | 15 | 20 | 10 | 15 | 10 | 25 |
| 50 | 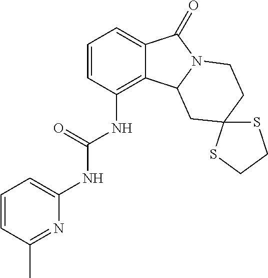 | 5 | 10 | 15 | 12 | 6 | 15 | 2 |
| 51 | 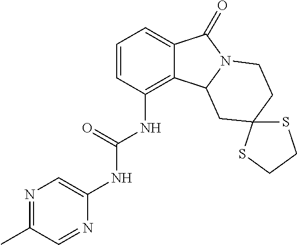 | 0.3 | 0.5 | 0.4 | 0.2 | 0.4 | 0.8 | 8 |
| 52 | 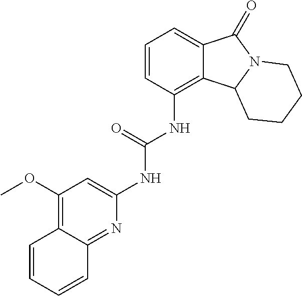 | 2 | 4 | 2 | 8 | 20 | 7 | 5 |
| 53 | 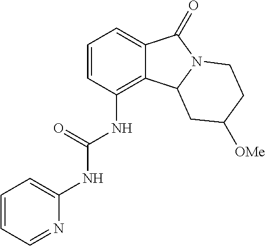 | 0.7 | 2.5 | 2 | 1 | 2 | 4 | 3 |
| 54 | 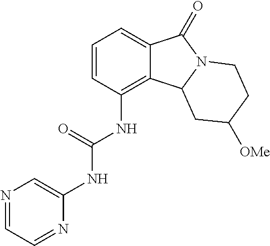 | 4 | 20 | 5 | 4 | 7 | 10 | 10 |
| 55 | 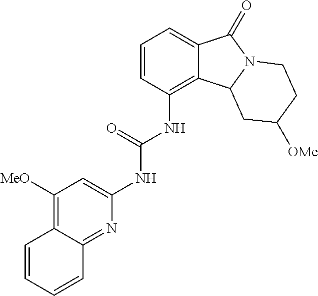 | 3 | 10 | 15 | 2.5 | 3 | 5 | 10 |
| 56 | 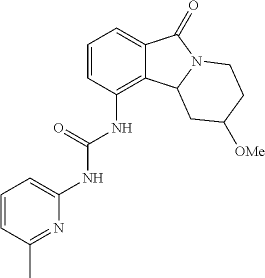 | 3 | 6 | 7 | 3 | 6 | 10 | 7 |
| 57 | 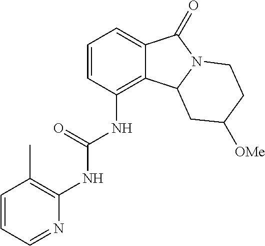 | 1.2 | 2 | 2.2 | 1 | 1.2 | 4 | 3 |
| 58 | 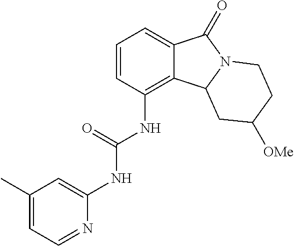 | 0.1 | 0.5 | 0.3 | 0.03 | 0.4 | 0.4 | 0.5 |
| 59 | 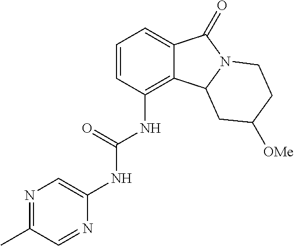 | 1.2 | 12 | 6 | 3 | 5 | 6 | 5 |
| 60 | 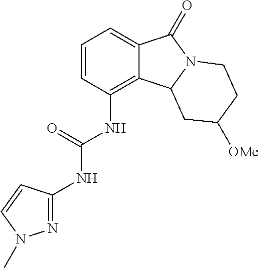 | 2 | 15 | 10 | 4 | 7 | 2 | 15 |
| 62 | 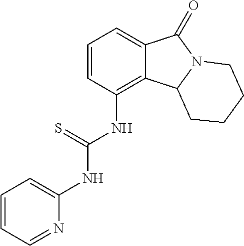 | >25 | 20 | 25 | 8 | 20 | >25 | >25 |
| 63 | 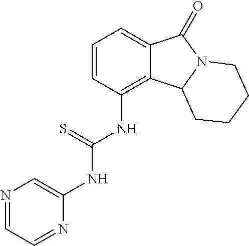 | >25 | >25 | >25 | >25 | >25 | >25 | >25 |
| 64 | 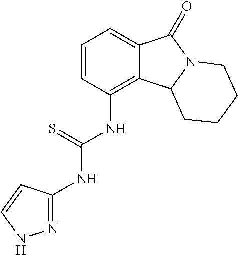 | 8 | 20 | 25 | 20 | 6 | 25 | >25 |
| 65 | 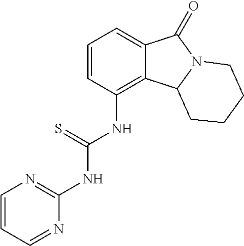 | >25 | 20 | 25 | >25 | >25 | >25 | >25 |
| 66 | 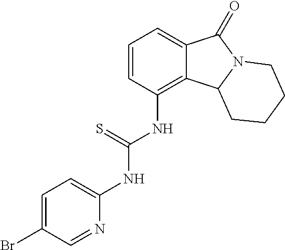 | >25 | 2 | 2 | 3 | >25 | 25 | 4 |
| 67 | 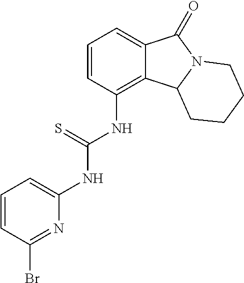 | 10 | 3 | 4 | 2 | 8 | 12 | 10 |
| 68 | 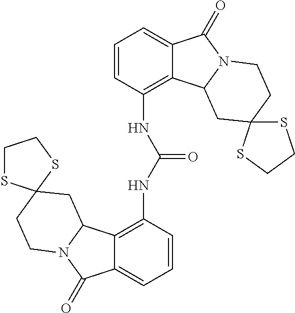 | 1 | >25 | >25 | 3 | 7 | 5 | >25 |
| 69 | 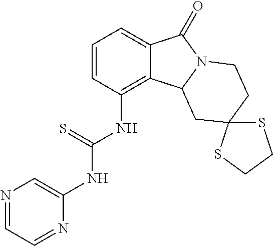 | >25 | 4 | 10 | 15 | >25 | >25 | 25 |
| 70 | 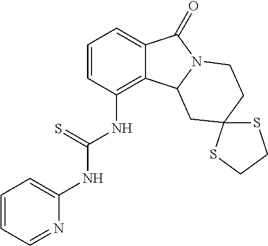 | 15 | 2 | 3 | 2 | >25 | 10 | 20 |
| 71 | 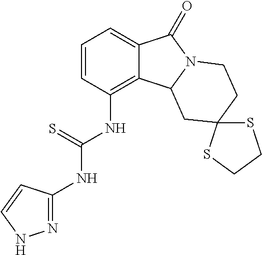 | 20 | 15 | 15 | 7 | >25 | 15 | 25 |
| 74 | 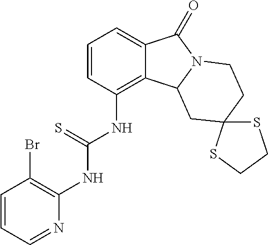 | 10 | 20 | >25 | 25 | 15 | 12 | 25 |
| 75 | 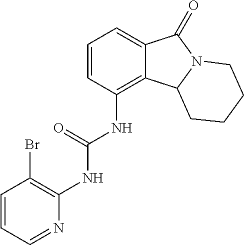 | 0.25 | 5 | 1.5 | 0.4 | 7 | 2 | 0.6 |
| 76 | 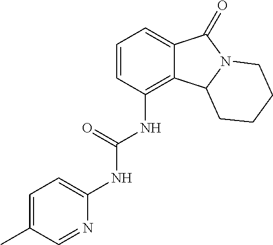 | 0.04 | <0.1 | <0.1 | <0.1 | <0.1 | <0.1 | <0.1 |
| 96 | 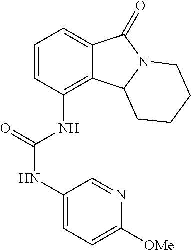 | 0.5 | 0.8 | 0.2 | 0.2 | 0.6 | 0.6 | 1.5 |
| 97 | 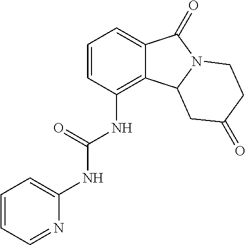 | 3 | 7 | 3 | 2 | 5 | 2 | 6 |
| 98 | 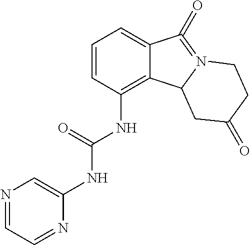 | >25 | 25 | >25 | 20 | >25 | >25 | >25 |
| 103 | 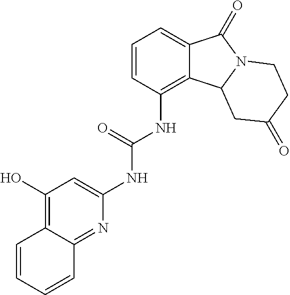 | | | | | | | |
| 104 | 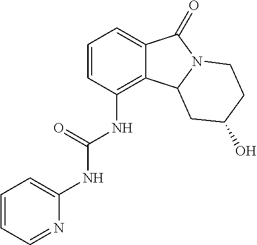 | 2 | 7 | 3 | 1 | 3 | 3 | 2 |
| 105 | 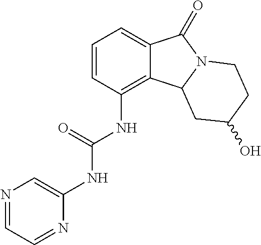 | 4 | >25 | 25 | 8 | 20 | 8 | 12 |
| 107 | 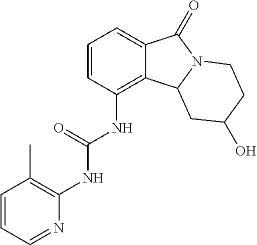 | 5 | 12 | 8 | 1.5 | 5 | 7 | 6 (50%) |
| 108 | 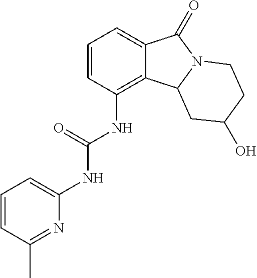 | 3 | 15 (brusque) | 8 | 3 | 2 | 10 | 10 |
| 109 | 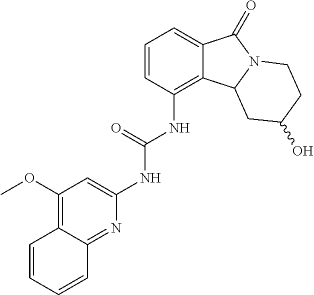 | 1 | 15 | 6 | 1 | 5 | 1 (50%) | 5 |
| 110 | 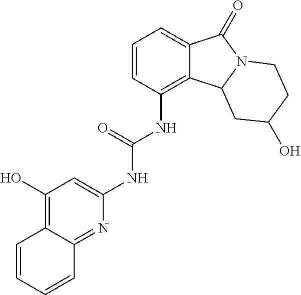 | | | | | | | |
| 118 | 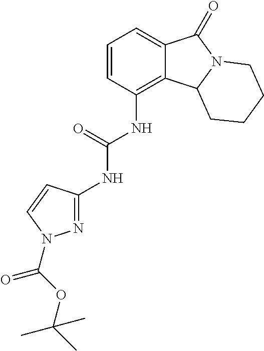 | | | | | | | |
| 119 | 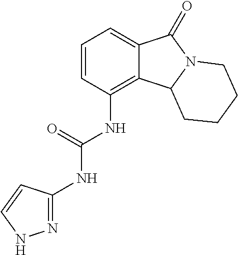 | | | | | | | |

| | IC₅₀ μM | | | |
|---|---|---|---|---|
| | Kinases | | | |
| Number | CDK1 | DYRK1A | CDK5 | GSK3 |
| 16 | 0.22 | >10 | 0.24 | 0.033 |
| 17 | 0.13 | >10 | 0.15 | 0.044 |
| 18 | ND | 21 | 0.13 | 0.076 |
| 19 | ND | 3.4 | 0.040 | 0.043 |
| 20 | ND | 9 | 0.4 | 0.022 |
| 21 | ND | 8.7 | >10 | 0.71 |
| 22 | ND | 4 | 0.096 | 0.042 |
| 23 | ND | 3.5 | 0.9 | 0.026 |
| 24 | ND | >10 | 2.7 | 0.033 |
| 25 | >10 | >10 | 1.5 | 0.27 |
| 26 | ND | >10 | 0.43 | 0.76 |
| 27 | ND | >10 | 0.04 | 0.02 |
| 28 | ND | 80 | 11 | 0.7 |
| 29 | ND | 93 | 7.1 | 0.26 |
| 30 | 0.075 | 27 | 0.08 | 0.17 |
| 31 | ND | ≧10 | 7.3 | 0.068 |
| 32 | ND | 71 | 1.5 | 0.36 |
| 33 | ND | >100 | 10 | 4.3 |
| 34 | ND | 88 | 16 | 3.5 |
| 35 | ND | >10 | 0.53 | 0.12 |
| 36 | ND | 2.2 | 0.035 | 0.0066 |
| 37 | ND | 2.4 | 1.7 | 0.13 |
| 38 | 0.07 | >100 | 0.07 | 0.51 |
| 39 | ND | >10 | 0.023 | 0.06 |
| 40 | ND | >10 | ≧10 | 1.9 |
| 41 | ND | >10 | 0.51 | 0.31 |
| 42 | ND | >10 | >10 | 0.75 |
| 43 | ND | >10 | 0.81 | 0.072 |
| 44 | ND | 4.5 | 0.16 | 0.17 |
| 45 | ND | >10 | 3.1 | 0.61 |
| 46 | ND | >10 | >10 | 0.92 |
| 47 | ND | >10 | >10 | 0.89 |
| 48 | ND | >10 | >10 | 1 |
| 49 | ND | >10 | >10 | 4 |
| 50 | ND | >10 | >10 | 2 |
| 51 | ND | >10 | 0.22 | 0.05 |
| 52 | ND | >10 | 0.15 | 0.08 |
| 53 | ND | >10 | 0.55 | 0.081 |
| 54 | ND | >10 | 0.48 | 0.2 |
| 55 | ND | >10 | 8.5 | 0.18 |
| 56 | ND | >10 | 4.3 | 0.32 |
| 57 | ND | >10 | 1.1 | 0.25 |
| 58 | ND | >10 | 0.32 | 0.032 |
| 59 | ND | >10 | 0.32 | 0.14 |
| 60 | ND | >10 | 2.3 | 1.1 |
| 62 | ND | 47 | 10 | 2.3 |
| 63 | ND | >100 | 26 | 8 |
| 64 | ND | ≧100 | 56 | 16 |
| 65 | ND | ≧100 | >10 | >10 |
| 66 | ND | >100 | 8.7 | 2.1 |
| 67 | ND | 92 | 8.1 | 4.5 |
| 68 | ND | >10 | >10 | >10 |
| 69 | ND | >10 | >10 | >10 |
| 70 | ND | 22 | 19 | 2.5 |
| 71 | ND | >10 | >10 | >10 |
| 74 | ND | >10 | >10 | 3 |
| 75 | ND | 3 | 0.48 | 0.17 |
| 76 | ND | >10 | 0.044 | 0.038 |
| 96 | 25 | 25 | 1.6 | 0.23 |
| 97 | ND | 50 | 3.6 | 1.2 |
| 98 | ND | >10 | >10 | 4.2 |
| 103 | ND | ≧10 | >10 | 1.3 |
| 104 | ND | 34 | 2.1 | 0.26 |
| 105 | ND | >10 | 0.084 | 0.032 |
| 107 | ND | 6.2 | 0.63 | 0.16 |
| 108 | ND | 7 | 1 | 0.03 |
| 109 | ND | >10 | 4.9 | 0.035 |
| 110 | ND | ≧10 | >10 | 2.5 |
| 118 | ND | >10 | 1.2/1.8 | 0.4 |
| 119 | ND | >10 | 0.17/0.26 | 0.47 |

1-14. (canceled)

15. A compound of the following general formula (I): 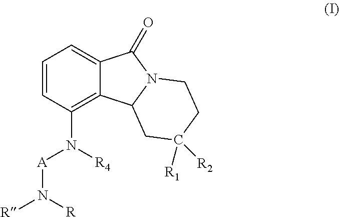
wherein:
A represents a group [symbol] 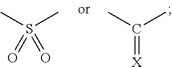
X representing O or S;
R″ represents H or an alkyl group comprising from 1 to 10 carbon atoms,
R₁ represents H and R₂ represents an hydrogen atom, an NR₃R′₃ group or an OR₃ group, R₃ and R′₃ representing independently of each other a hydrogen atom or an alkyl group comprising from 1 to 10 carbon atoms;
or R₁ and R₂ form together with the carbon atom on which they are bound,
a group 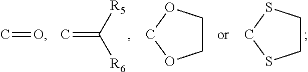
R₅ and R_{δ}representing independently of each other a hydrogen atom or an alkyl group comprising from 1 to 10 carbon atoms;
R₄ represents a hydrogen atom or an alkyl group comprising from 1 to 10 carbon atoms;
R represents an alkyl group comprising from 1 to 10 carbon atoms, an aryl group comprising from 6 to 30 carbon atoms or a cycloalkyl group comprising from 3 to 20 carbon atoms, said aforementioned alkyl, aryl or cycloalkyl groups optionally comprising one or more heteroatoms, optionally substituted, or when A is a —CO— group, R and R₄ may form together with the nitrogen atoms on which they are attached, a ring of the following formula (II): 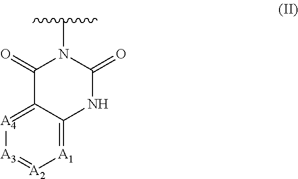
wherein one of the atoms among A₁, A₂, A₃ and A₄ represents N, and the three other atoms among A₁, A₂, A₃ and A₄ represent CH, as well as its pharmaceutically acceptable salts,
said compound of formula (I) being in the form of a pure stereoisomer or in the form of a mixture of enantiomers or diastereoisomers including racemic mixtures.

16. The compound according to claim 15, wherein R represents an aryl group selected from the group formed by phenyl, benzyl, pyridinyl, pyrimidyl, pyrazinyl, triazinyl, pyrazolyl, isoxazolyl, thiazolyl, benzothiazothiazolyl and quinolinyl groups optionally substituted or in that R represents cyclohexyl or piperidinyl, optionally substituted.

17. The compound according to claim 15, wherein R is selected from one of the following groups: 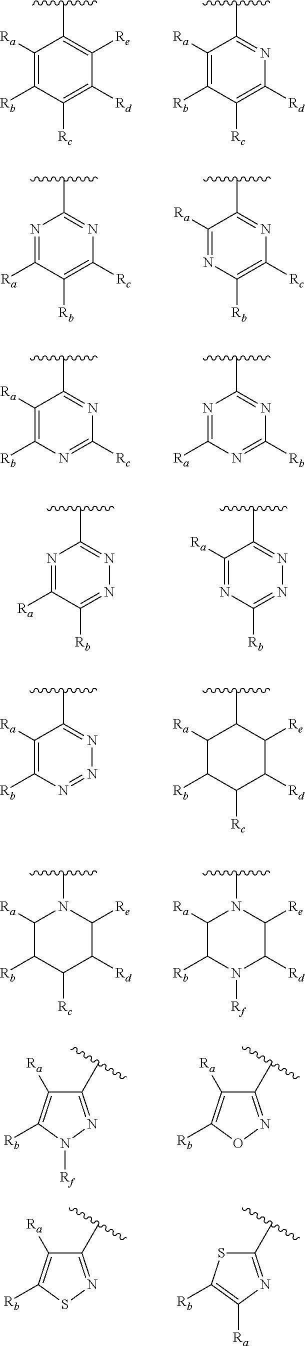 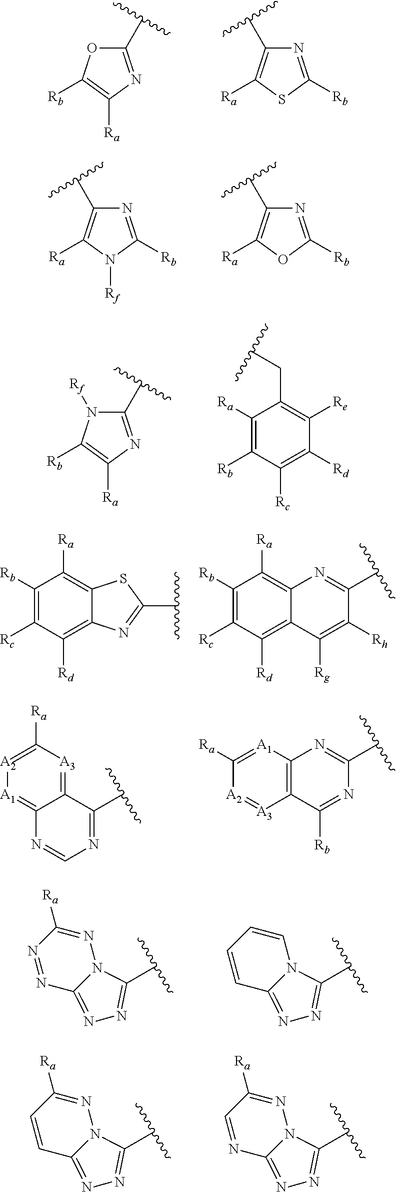
the groups Rₐ, R_{b}, R_{c}, R_{d}, Rₑ, R_{g} and Rₕ being selected independently of each other from the group formed by the following substituents:
a hydrogen atom,
a halogen atom
an alkyl group comprising from 1 to 10 carbon atoms, said alkyl group being optionally substituted with one or more substituents selected from the group formed by the following substituents:
halogen atoms,
alkenyl or alkynyl groups comprising from 2 to 10 carbon atoms,
aryl groups comprising from 6 to 30 carbon atoms,
COR_{α}, COOR_{α}, SR_{α}, OR_{α} or NR_{α}R_{β} groups, R_{α} and R_{β} representing independently of each other a hydrogen atom, an alkyl group compromising from 1 to 10 carbon atoms or an aryl group comprising from 6 to 30 carbon atoms,
a —CHO group,
a —CN group,
a phenyl group,
a —SR_{α} or OR_{α}group, R_{α} being as defined above
a —NR_{α}R_{β} group, R_{α} and R_{β} being as defined above
a —CONR_{α}R_{β} group, R_{α} and R_{β} being as defined above
a —NHCOR_{α} group, R_{α}being as defined above, and
a 2-pyridinyl group,
one of the atoms among A₁, A₂ and A₃ representing N, and the two other atoms from A₁, A₂ and A₃ representing CH,
the R_{f} group being a hydrogen atom, an alkyl group comprising from 1 to 10 carbon atoms.

18. The compound according to claim 15, fitting the following general formula (I-1-a): 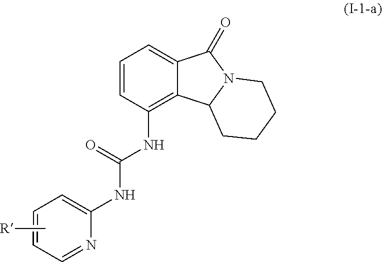
R′ represents a hydrogen atom, a halogen atom such as Br or F or an alkyl group comprising from 1 to 10 carbon atoms.

19. A drug comprising a compound of general formula (I) according to claim 15.

20. A method of inhibition of CDK1, CDK5 and/or GSK3 kinases, comprising a step of administering to a patient in need thereof a compound of general formula (I) according to claim 15.

21. A method for treating or preventing a disease related to deregulation of CDK1, CDK5 and/or GSK3 kinases, comprising a step of administering to a patient in need thereof a compound of general formula (I) according to claim 15.

22. A method for preparing a compound of general formula (I) according to claim 15, wherein R₄ and R″ are H and A is a CO group, and R₁ and R₂ are H or form together with the carbon atom to which they are attached,
a group 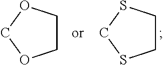
said method comprising:
a reaction step of the amine of the following formula: 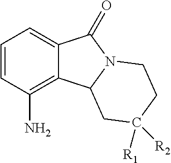
with an isocyanate of formula RNCO, R being as previously defined.

23. A method for preparing compound of general formula (I) according to claim 15, wherein R₄ and R″ are H and A is a CS group, and R₁ and R₂ are H or form together with the carbon atom to which they are attached, a group 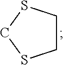
said method comprising:
a reaction step of the isothiocyanate of the following formula: 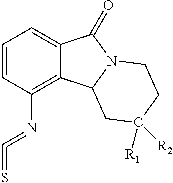
with an amine of formula RNH₂, R being as previously defined.

24. A method for preparing a compound of general formula (I) according to claim 15, wherein R₄ and R″ are H and A is a CO group, and R₁ and R₂ are H or form together with the carbon atom to which they are attached, a group 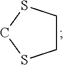
said method comprising:
a reaction step of the thiourea of the following formula: 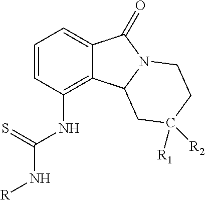
R being as previously defined,
in the presence of a CH₃CN/water mixture and of HgO at room temperature for a duration comprised from 24 to 40 hours.

25. A method for preparing a compound of general formula (I) according to claim 15, wherein R₄ and R″ are H and A is a CO group CO, and R₁ and R₂ are H or form together, with the carbon atom to which they are attached, a group 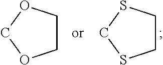
said method comprising:
a reaction step of the amine of the following formula: 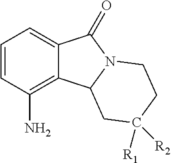
with an amine of formula RNH₂ and triphosgen, R being as previously defined.

26. A method for preparing a compound of general formula (I) according to claim 15, wherein R₁ and R₂ form together, with the carbon atom to which they are attached, a C═O group;
said method comprising:
a reaction step of the acetal of the following formula: 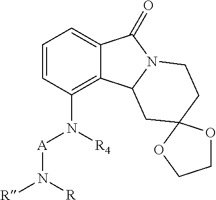
A, R, R″ and R₄ being as previously defined,
with acetone and hydrochloric acid.

27. A method for preparing a compound of general formula (I) according to claim 15, wherein R₁ is H and R₂ is OH,
said method comprising:
a reduction step of the ketone of the following formula: 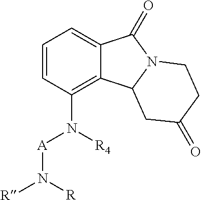
A, R, R″ and R₄ being as previously defined.

28. Intermediate compounds fitting one of the following formulae:

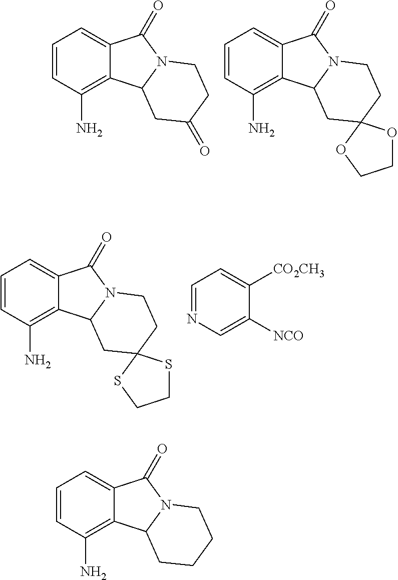

29. A method for treating or preventing a disease, comprising a step of administering to a patient in need thereof a compound of general formula (I) according to claim 15, wherein said disease is selected from the group consisting of cancers, Alzheimer's disease, Parkinson's disease, brain traumas, strokes, renal polycystosis, amyotrophic lateral sclerosis, viral infections, autoimmune diseases, neurodegenerative disorders, psoriasis, asthma, atypical dermatitises and glomerulonephrites.
